(19) United States
(12) Patent Application Publication
(10) Pub. No.: US 2017/0292961 A1
(43) Pub. Date: Oct. 12, 2017

(54) SYSTEMS AND METHODS FOR ASSESSING INTER-CELL COMMUNICATION

(71) Applicant: Q-STATE BIOSCIENCES, INC., Cambridge, MA (US)

(72) Inventors: Adam Cohen, Cambridge, MA (US); Kevin C. Eggan, Boston, MA (US); Joel Kralj, Somerville, MA (US); Daniel Hochbaum, Cambridge, MA (US); Graham Dempsey, Cambridge, MA (US)

(21) Appl. No.: 15/515,027

(22) PCT Filed: Oct. 2, 2015
(86) PCT No.: PCT/US2015/053721

§ 371 Date: Mar. 28, 2017

Related U.S. Application Data
(60) Provisional application No. 62/058,943, filed on Oct. 2, 2014.
(60) Provisional application No. 62/058,935, filed on Oct. 2, 2014.

(51) Int. Cl. G01N 33/68; G01N 33/483; G01N 33/50; C12Q 1/68

(52) U.S. Cl. CPC G01N 33/6872; C12Q 1/6897; G01N 33/4833; G01N 33/5041; G01N 33/5061; G01N 33/5058; G01N 2333/405

(57) ABSTRACT

The invention relates to methods of assessing communication between cells. Methods of the invention use optical reporters of cellular electrical activity to evaluate signal propagation between cells and can be used to study an individual synapse or a complex network of interconnected cells. Aspects of the invention provide a method for characterizing signal propagation between cells. The method includes providing a first cell containing a light-generating reporter and a second cell, in which the first cell and the second cell are in communication. The second cell may contain an optical actuator of cellular electrical activity. The second cell is exposed to a stimulus and an optical signal from the first cell is detected.

### CROSS-REFERENCE TO RELATED APPLICATION

This applications claims priority to and the benefit of U.S. Provisional Patent Application No. 62/058,935, filed Oct. 2, 2014, and to U.S. Provisional Patent Application No. 62/058,943, filed Oct. 2, 2014, the contents of each of which are incorporated by reference.

### FIELD OF INVENTION

The invention relates to methods of assessing communication between cells.

### BACKGROUND

Some debilitating diseases are associated with a breakdown in cellular communication. For the heart to beat, cardiac muscle cells must receive and propagate electrical signals. Needless to say, failure of those functions can be fatal. Similarly, the human mind operates via a complex network of signals. Conditions such as Parkinson's and Alzheimer's involve the deterioration of mental function with unfortunate consequences for the affected person.

Existing approaches to studying cellular communication are unsatisfactory. Animal models are difficult to work with and may not be applicable to humans. For example, a misfolded protein that is fatal to humans may be inconsequential to a mouse. In vitro assays are limited to studying single cells. Thus, it can be very difficult to discover and understand the mechanisms of diseases that affect systems such as the heart or mind.

### SUMMARY

Methods of the invention use optical reporters of cellular electrical activity to evaluate signal propagation between cells. The methods can be used in vitro with electrically active cells such as neurons and cardiomyocytes. The cells may be obtained by taking somatic cells from a person and using stem cell engineering techniques to convert somatic cells to specific cell types. Two or more cells can be provided that are poised to communicate with one another. An optical reporter in one of the cells can reveal if that cell exhibits electrical activity in response to a signal from another cell. The methods can be used to study an individual synapse or a complex network of interconnected cells. Several samples that each include interconnected cells can be assayed in parallel and cellular samples can be used to screen for the effects of compounds or treatments on cellular communication. Where samples are derived from a patient, the cells are isogenic with that patient, providing the potential for truly personalized medicine. Methods of the invention can illustrate the function or dysfunction of inter-cellular communication providing insights into mechanisms that underlie diseases. Methods can also be used to study the effects of compounds on cellular communication and could help discover compounds that counteract the harm cause by certain diseases. Since methods of the invention can be used to study disease mechanisms or discover treatments for those diseases, they may be used to provide cures for significant diseases that threaten human health. Since the analyzed cells can be obtained from a patient, the prospective manifestation of a suspected disease or the effects of a drug can be studied for that patient personally. For such applications the invention provides in vitro models of functioning neural and cardiac networks.

In certain aspects, the invention provides a method for analyzing cellular signaling. The method includes providing a first cell comprising an optical actuator and providing a second cell in communication with the first cell, the second cell comprising an optical voltage reporter. Further, the method includes exposing the first cell to a stimulus, detecting an optical signal from the second cell, and evaluating the optical signal, thereby characterizing signal propagation from the first cell to the second cell. Preferably, the optical actuator is a light-gated ion channel and the optical voltage reporter is a microbial rhodopsin. The stimulus is illumination and the detected optical signal results at least in part from an action potential propagating in the second cell. In some embodiments, the illumination is spatially-resolved to specifically target the first cell. Spatial resolution may be provided using a digital micromirror device. The second cell is preferably a neuron or a cardiomyocyte.

In a preferred embodiment, the first cell and the second cell are among a cluster of neurons when exposed to the stimulus, and detecting the optical signal includes using a microscope to detect a plurality of signals from the cluster of cells and using a computer system to isolate the optical signal of the second cell from among the plurality of signals. The computer system isolates the optical signal by performing an independent component analysis and identifying a spike train from the second cell. In certain embodiments, the second cell also comprises an optical reporter of intracellular calcium such as a GCaMP variant. Preferably the optical reporter of intracellular calcium and the optical voltage reporter are provided together as a fusion protein. This ensures that all of the cells observed using the microscope include equal amounts of calcium and voltage reporters allowing for good comparisons between signals from different times or different cells. In some embodiments, the light-gated ion channel used to initiate the signal propagation is an algal channelrhodopsin. Additionally or alternatively, detecting the signal may include capturing fluorescence from a plurality of cells on a non-imaging detector such as a photomultiplier tube, a photodiode, or an avalanche photodiode.

In the preferred embodiment, the method may include detecting a change in AP waveform and a change in the intracellular calcium level for the second cell upon exposing the first cell to the stimulus. The method may include obtaining a sample cell from a person, converting the sample cell into the second cell, and providing the second cell with the microbial rhodopsin. For example, a somatic cell may be converted into a specific neuronal type via direct reprogramming or through a stem cell intermediary.

The method may include performing the same steps on a second sample for a control, i.e., providing a third cell comprising the optical actuator and providing a fourth cell in communication with the third cell, the fourth cell comprising an optical voltage reporter, and further wherein the fourth cell comprises a genetic mutation relative to the second cell. For the second sample, the method includes exposing the third cell to a stimulus, detecting an second optical signal from the fourth cell (e.g., wherein the optical signal and the second optical signal represent changes in membrane potential and intracellular calcium levels), and comparing—using the computer system—the second optical signal to the optical signal to determine an effect of the genetic mutation on signal propagation. Cells in the control sample and in the second sample may differ in a controlled way. For example, the second cell and the fourth cell may be derived from the same donor person or animal, but one may have a mutation. The mutation may be introduced specifically using, for example, a genome editing system such as CRISPR/Cas9, Cpf1, Fok1, or the like. Preferably, the signal from the optical voltage reporter comprises light that does not stimulate the first cell.

In certain embodiments, the cells may include cells that do not endogenously produce action potentials, but which have been genetically modified to express one or more ion channels which imbue the cells with electrical spiking behavior. Human Embryonic Kidney (HEK) cells are an example of such cells. These cells can be modified to express an inward rectifier potassium channel and a voltage-gated sodium channel, whereupon they develop the ability to produce action potentials. See Jeehae et al., 2013, Screening fluorescent voltage indicators with spontaneously spiking HEK cells, PLoS one 8.12:e85221, incorporated by reference. Such cells could be used in screens to detect the effect of pharmacological agents that modify the activity of one or more of the heterologously expressed ion channels.

Aspects of the invention provide a method for characterizing signal propagation between cells. The method includes modifying a first cell to include a light-activated actuator, modifying a second cell to include a light generating reporter, and activating the light-activated actuator when the first cell is in proximity to the second cell. An optical signal from the second cell is detected and evaluated with respect to the first light, thereby characterizing propagation of a signal from the first cell to the second cell. That actuator, reporter, or both can be provided by rhodopsin-based constructs described herein. The actuator can be stimulated by illuminating the cells. The stimulating light and the optical signal can be spectrally orthogonal such that the optical signal from the second cell does not stimulate the first or second cell. The method may include exposing the first cell, the second cell, or both to an agent such as an ion, a molecule, a compound, an element, an antibody, or a nucleic acid.

Aspects of the invention provide a method for characterizing signal propagation between cells. The method includes providing a first cell containing a light-generating reporter and a second cell, in which the first cell and the second cell are in communication. Preferably, the second cell contains an optical actuator of cellular electrical activity. The second cell is exposed to a stimulus and an optical signal from the first cell is detected. The method includes evaluating the optical signal, thereby characterizing propagation of a cellular signal from the first cell to the second cell. The cell containing the light-activated actuator and the cell containing the light generating reporter may be in direct synaptic communication or in synaptic communication through at least one intermediate cell. In certain embodiments, the light-activated actuator initiates an action potential in response to the stimulus. The stimulus can be illumination, e.g., as provided using spatially resolved light from a digital micromirror. Preferably the signal from the light-generating reporter comprises light that does not stimulate the cell. Illuminating the cells and obtaining the signal may be done simultaneously. Any suitable cell type can be included such as neurons, cardiac cells, glial cells, or genetically engineered HEK cells (e.g., see Jeehae 2013). The method may include exposing the cells to an agent and optionally repeating the exposing, detecting, and evaluating steps before and after exposing the cells to the agent. Cells can include mutations and the method can reveal effects of the mutations on the cells. In some embodiments, detecting the signal includes observing a cluster of different cells with a microscope and using a computer to isolate the signal generated by the optical reporter from a plurality of signals from the different cells. The computer may isolate the signal by performing an independent component analysis and identifying a spike train associated with the cell. A microscope to obtain an image of a plurality of clusters of cells.

Aspects of the invention provide a method for screening compounds by arranging a plurality of samples on a substrate, wherein one or more of the samples includes a network of cells in communication with each other, and in which the cells use an optical reporter of, and optionally an optical actuator of, electrical activity. The method includes exposing at least one sample to at least one agent, stimulating the cells, and detecting a signal from the optical reporter. The detected signal can be analyzed and a response of a cell to exposure to an agent can be characterized. The cells can be stimulated via the optical actuator. The cells can be exposed to single or multiple stimuli. The substrate may be a microtitre plate with a plurality of wells. Signals from the optical reporter can be measured over time, continuously or discretely, over a span of a day, week, month, etc.

In some embodiments, subsets of the plurality of samples are exposed to different agents. A subset of the samples can be not exposed to the agent. A subset can be exposed to multiple agents. Any of the samples can be exposed to changing concentrations of an agent. Cells can be monitored for certain responses such as apoptosis. The exposure and detection can be repeated (e.g., over the same sample or over numerous samples) to support a statistical significance of a relationship between exposure to an agent and an exhibited cellular response.

The samples may include various cell types such as liver cells, lung cells, pancreatic cells, kidney cells, stomach cells, dermal cells, neurons and cardiac cells.

Methods of the invention are suited to high-throughput workflows, thereby potentially speeding drug discovery and development. A shortened drug discovery pipeline speeds a drug to market, benefiting and saving many patient lives. Compounds can be screened by evaluating the signals detected from the optical reporter. Clusters or subsets of the cells are arranged in arrays for parallel testing, with different clusters or subsets being exposed to different agents or combinations of agents. Principles of combinatorial chemistry can be used to test multiple agents in combination. Libraries of numerous agents are applied to comprehensively identify and characterize their effects on electrical or chemical communication between cells. In addition, cluster or subsets within the array can also include cells from various organs to characterize an agent's impact over a wide variety of cells. Compounds may be screened in combination with other drug agents to construct interference profiles.

Methods of the invention are useful for investigating neural networks. Activity of a neuron is not simply a function of its local receptive field or properties, but depends on a wide array of stimuli. The activity of surrounding neurons and activity from outside the brain can influence the activity of a neuron. Methods of the invention can be used to study not only the communication between two neurons, but communication among a plurality of neurons.

Using the optical methods of the present invention, an optical signal is detected when a signal from one cell and received by another cell. There may be intermediate cells, and a signal that propagates over several cells can be detected or followed. By such means, a network is probed for depth or length of signal propagation. Signals from the optical reporters may be correlated to values of membrane potential. The signal may give a probability of a voltage spike in response to the stimulation of the cell or a change in such probability relative to a control. Where numerous convoluted signals are obtained in a single imaging or detection operation, individual signals can be resolved from the convoluted signals using methods herein. Obtaining the signal may include observing a cluster of different cells with a microscope and using a computer to isolate the signal generated by one cell from a plurality of signals from different cells. An independent component analysis may be used to identify a signal or spike train associated with the cell. A microscope of the invention may be used to obtain an image of a plurality of clusters of cells. Unlike other systems that require one image per cluster or per cell, a wide field microscope system with signal deconvolution can image a plurality of cells or clusters per image useful for high-throughput assays of multiple targets in parallel.

### BRIEF DESCRIPTION OF THE DRAWINGS

FIG. 1 illustrates a method for characterizing a cell.

FIG. 2 illustrates exemplary pathways for converting cells into specific neural subtypes.

FIG. 3 gives an overview of a method for genome editing.

FIG. 4 shows genetically encoded fluorescent voltage indicators classified according to their sensitivity and speed.

FIG. 5 shows the dependence of fluorescence on membrane voltage of Archaerhodopsin-based voltage indicators.

FIG. 6 shows the response of fluorescence to a step in membrane voltage of Archaerhodopsin-based voltage indicators.

FIG. 7 shows whole-cell membrane potential determined via electrical recording.

FIG. 8 shows optical recordings using a QuasAr.

FIG. 9 shows average waveforms captured using a QuasAr.

FIG. 10 gives a functional diagram of components of an optical imaging apparatus.

FIG. 11 illustrates a pulse sequence of red and blue light used to record action potentials.

FIG. 12 shows an image that contains five neurons whose images overlap.

FIG. 13 shows clusters of pixels whose intensity varies synchronously found by an independent component analysis (ICA).

FIG. 14 illustrates contributions from individual cells to the ICA time course.

FIG. 15 shows an overlay of filters used to map individual cells in an image.

FIG. 16 shows a patterned optical excitation being used to induce action potentials.

FIG. 17 shows eigenvectors resulting from a principal component analysis of a single action potential waveform.

FIG. 18 shows a relation between cumulative variance and eigenvector number for the principal component analysis of FIG. 17.

FIG. 19 compares action potential waveforms before and after smoothing operations.

FIG. 20 shows an action potential timing map.

FIG. 21 shows the accuracy of timing extracted by a sub-Nyquist action-potential timing (SNAPT) algorithm.

FIG. 22 gives an image of eGFP fluorescence, indicating CheRiff distribution in a neuron.

FIG. 23 presents frames from a SNAPT movie.

FIG. 24 illustrates an output from measuring action potentials in hiPSC-derived motor neurons containing a mutation associated with amyotrophic lateral sclerosis.

FIG. 25 demonstrates effects of dimethyl sulfoxide (DMSO) on hiPSC-derived cardiomyocytes action potential waveform.

FIG. 26 presents the effects of DMSO control vehicle and pacing rate on the average action potential waveform.

FIG. 27 presents the effects of DMSO control vehicle and pacing rate on the average rise time.

FIG. 28 shows the dose dependent response of action potential width at 50% repolarization (AP50) to increasing concentrations of DMSO.

FIG. 29 shows the dose dependent response of action potential rise time to increasing concentrations of DMSO FIG. 30 shows the dose dependent response of action potential width at 90% repolarization (AP90) to increasing concentrations of DMSO.

FIG. 31 shows the dose dependence of the spontaneous beat rate as a function of DMSO concentration.

FIG. 32 shows models of Optopatch and CaViar.

FIG. 33 shows absorption and fluorescence emission spectra.

FIG. 34 top shows a HEK cell expressing Arch, visualized via Arch fluorescence.

FIG. 35 shows fluorescence of Arch as a function of membrane potential.

FIG. 36 shows dynamic response of Arch to steps in membrane potential.

FIG. 37 shows sensitivity of Arch 3 WT to small steps in membrane voltage.

FIG. 38 shows that Arch 3 reports action potentials without exogenous retinal.

FIG. 39 presents a system useful for performing methods of the invention.

FIG. 40 diagrams a microscopy setup for illuminating the cellular sample.

FIG. 41 illustrates the action potentials obtained by exposing cardiomyocytes to a compound that blocks hERG trafficking.

FIG. 42 shows results from exposing cardiomyocytes to a hERG channel blocker.

FIG. 43 gives the average waveform and rise times after exposures.

FIG. 44 gives summary statistics for freely beating cardiomyocytes and paced cardiomyocytes upon exposure to a compound.

FIG. 45 shows results from exposing cardiomyocytes to a channel blocker.

FIG. 46 gives the average waveform and rise times.

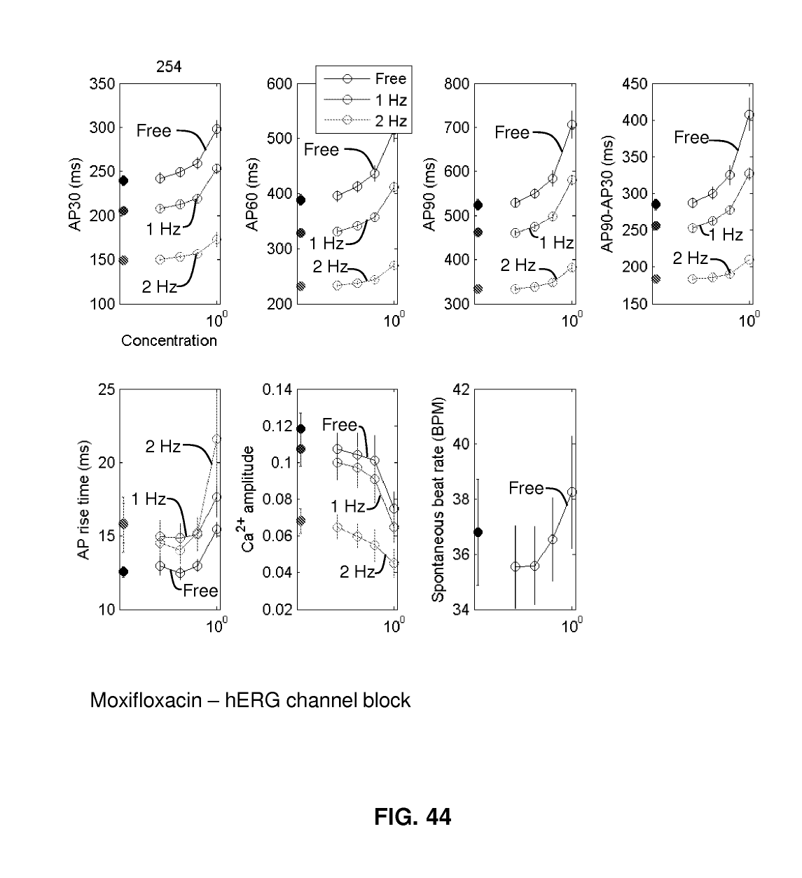

FIG. 44 gives summary statistics for freely beating cardiomyocytes and paced cardiomyocytes upon exposure to different concentrations of flecainide.

FIG. 48 shows the action potential timing for a number of neurons.

FIG. 49 plots the firing rate s before and after exposure to tetraethylammonium.

FIG. 50 graphs spike frequency over unitless scaled stimulus power.

FIG. 51 presents shape parameters of the waveforms for the neurons.

FIG. 52 shows results of exposing neurons to acute doses of an anticonvulsant.

FIG. 53 shows results of exposing neurons to chronic doses of an anticonvulsant.

### DETAILED DESCRIPTION

The invention relates generally to optical detection of cellular communication within a network, or network effects of cells. Optical detection of a signal that has propagated between two cells allows for the investigation of synaptic communication. In some embodiments, individual cells or the entire network of cells are exposed to agents and synaptic communication is monitored for impact or alterations attributable to the agents.

Systems and methods of the invention use optical actuators and optical detectors to study network effects. One set of cells may only contain the actuator, with another set of cells only containing the reporter. In preferred embodiments, if a cell or set of cells contains an actuator, the cells do not contain the reporter. Likewise, if a cell or set of cells contains a reporter, the cells do not contain an actuator. The reporter will only emit an optically detectable signal if a proximate cell containing an actuator is stimulated and the signal propagates between the cells. This ability to probe network effects allows for investigations of disease states, and possible drug or compound impact on cell-cell communications.

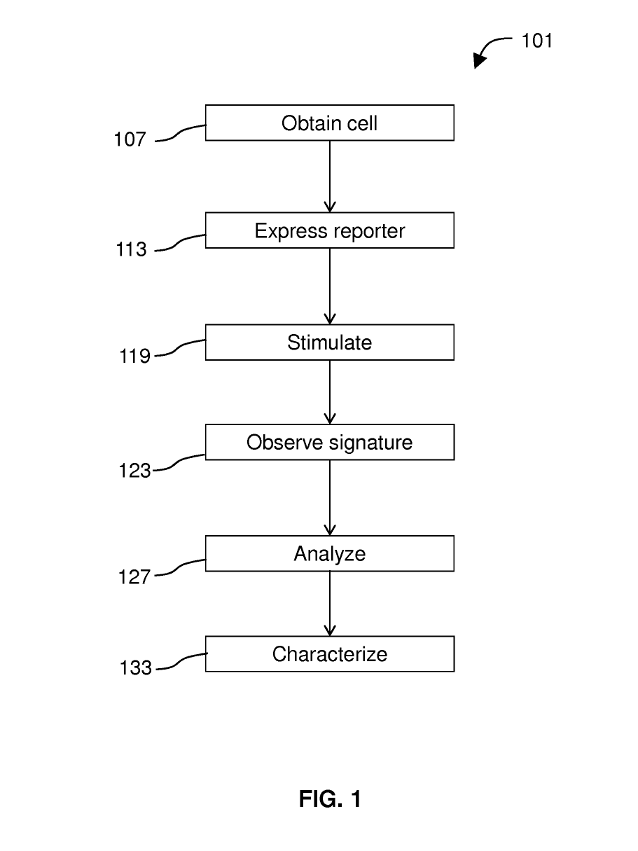

FIG. 1 illustrates a method 101 to characterize 133 a cell. Methods are given to obtain 107 an electrically excitable cell. An optical reporter of electrical activity is incorporated into the cell. In preferred embodiments, only an actuator or a reporter is incorporated into the cell. Preferably, the cell will express 113 (e.g., by translation) the reporter. An optical signal from the optical reporter in response to a stimulation of the cell is obtained. To characterize the cell, one may observe 123 a signature of the signal and analyze or evaluate 127 the signature. By evaluating the signal, one may characterize 133 the cell.

Methods of the invention may involve any type of cell, for example, neurons, cardiomyocytes, cardiac pacemaker cells, etc. Cells communicate with each other via direct contact (juxtacrine signaling), over short distances (paracrine signaling), or over large distances and/or scales (endocrine signaling). Methods of the invention may be customized to investigate particular cells, and may probe various configurations of signaling distances. Some cell-cell communication requires direct cell-cell contact. Some cells can form gap junctions that connect their cytoplasm to the cytoplasm of adjacent cells. For example, in cardiac muscle, gap junctions between adjacent cells allows for action potential propagation from the cardiac pacemaker region of the heart to spread and coordinately cause contraction of the heart. Methods of the invention allow for cells to be positioned achieving cell-cell communication.

The nervous system is formed by two major cell types, neurons and glial cells. Methods of invention may incorporate neurons or glial cells, or both in combination. Glial cells surround neurons and provide support for and insulation between them. Glial cells are the most abundant cell types in the central nervous system and are subdivided into different types with different functions: oligodendroglia, microglia, ependimoglia and astroglia. Astrocytes are known to play important roles in the homeostasis of the extracellular environment, providing the adequate conditions for the appropriate function of neurons and synapses. Astrocytes can dynamically shape the extracellular space, which may have a strong impact on the neuronal network by influencing the extracellular diffusion of neurotransmitters. See Syková E, 2008 Diffusion in brain extracellular space, Physiol. Rev. 88:1277-1340. Modifications of the astrocytic sheathing of synapses that occur under specific physiological conditions strongly influence synaptic efficacy, owing to changes in the effectiveness of glutamate clearance. See Oliet et al., 2001, Control of glutamate clearance and synaptic efficacy by glial coverage of neurons, Science 292:923-926.

Intercellular signaling may be not between just neurons, but bidirectional signaling between neurons and astrocytes. While neurons base their cellular excitability on electrical signals generated across the membrane, astrocytes base their cellular excitability on variations of the Ca²⁺ concentration in the cytosol. These Ca²⁺ variations may serve as an intracellular and intercellular signal that can propagate within and between astrocytes, signaling to different regions of the cell and to different cells. See Perea, 2005, Properties of synaptically evoked astrocyte calcium signal reveal synaptic information processing by astrocytes, J. Neurosci. 25:2192-2203. The calcium-based cellular excitability displayed by astrocytes can be triggered by neuronal and synaptic activity through activation of neurotransmitter receptors expressed by astrocytes. In turn, astrocyte calcium elevations stimulate the release of different neuroactive substances—called gliotransmitters—such as glutamate, ATP and d-serine, which regulate neuronal excitability and synaptic transmission. See Haydon, 2002, GLIA: listening and talking to the synapse, Nat. Rev. Neurosci. 2:185-193. These findings have led to the establishment of a new concept in synaptic physiology, the tripartite synapse, in which astrocytes exchange information with the neuronal synaptic elements. See Araque et al., 1999, Tripartite synapses: glia, the unacknowledged partner, Trends Neurosci. 22:208-215.

Methods of the invention may investigate neurons in isolation from glial cells, or in combination with glial cells. Either cell may be modified to contain either an actuator or a reporter of the invention. In some embodiments, only neurons may contain actuators and reporters, while in other embodiments, glial cells may contain actuators and reporters.

Methods of the invention may incorporate cardiac cells, investigating the propagation of signals among these types of cells. There are two types of cells within the heart: the cardiomyocytes and the cardiac pacemaker cells. Cardiomyocytes are the muscle cells that make up the cardiac muscle. However, the heart's remarkable degree of regularity and adaptability is controlled by a pacemaker system located in the sinoatrial node pacemaker cell that generates the repetitive action potentials that travel through gap junctions to excite all the contractile cells to drive each heartbeat. This communication through electrical signals is used to spread the action potentials from the sinoatrial node throughout the atrium, where it triggers atrial cell contraction. The action potential then invades the atrial-ventricular node that is coupled to the Purkinje fibres responsible for transmitting action potentials to the ventricles to stimulate ventricular cell contraction. The cardiac action potential differs from the neuronal action potential by having an extended plateau, in which the membrane is held at a high voltage for a few hundred milliseconds prior to being repolarized by the potassium current as usual.

Methods of the invention may incorporate muscle cells. Muscle action potentials are provoked by the arrival of a pre-synaptic neuronal action potential at the neuromuscular junction, which is a common target for neurotoxins. The action potential in a normal skeletal muscle cell is similar to the action potential in neurons. The muscle action potential lasts roughly 2-4 ms, the absolute refractory period is roughly 1-3 ms, and the conduction velocity along the muscle is roughly 5 m/s. The action potential releases calcium ions that free up the tropomyosin and allow the muscle to contract.

Methods of the invention may incorporate plant and/or fungal cells that are electrically excitable. The depolarization in plant cells is not accomplished by an uptake of positive sodium ions, but by release of negative chloride ions. The interaction of electrical and osmotic relations in plant cells may be investigated and characterized using the methods of the present invention. The cells can also be Gram positive or a Gram negative bacteria, as well as pathogenic bacteria of either Gram type. The pathogenic cells are useful for applications of the method to, e.g., screening of novel antibiotics that affect membrane potential to assist in destruction of the bacterial cell or that assist destruction of the bacterial cell in combination with the membrane potential affecting agent; or in the search for compounds that suppress efflux of antibiotics.

In some embodiment, the cell is an “artificial cell” or a “synthetic cell” created by bioengineering. See Gibson et al., 2010, Creation of a Bacterial Cell Controlled by a Chemically Synthesized Genome, Science 329(5987):52-56 and Cans et al., 2008, Positioning Lipid Membrane Domains in Giant Vesicles by Micro-organization of Aqueous Cytoplasm Mimic, J Am Chem Soc 130:7400-7406.

The methods can also be applied to any other membrane-bound structure, which may not necessarily be classified as a cell. Such membrane bound structures can be made to carry the microbial rhodopsin proteins of the invention by, e.g., fusing the membranes with cell membrane fragments that carry the microbial rhodopsin proteins of the invention.

The membrane potential of essentially any cell, or any phospholipid bilayer enclosed structure, can be measured using the methods and compositions described herein. Examples of the cells that can be assayed are a primary cell e.g., a primary hepatocyte, a primary neuronal cell, a primary myoblast, a primary mesenchymal stem cell, primary progenitor cell, or it may be a cell of an established cell line. It is not necessary that the cell be capable of undergoing cell division; a terminally differentiated cell can be used in the methods described herein. In this context, the cell can be of any cell type including, but not limited to, epithelial, endothelial, neuronal, adipose, cardiac, skeletal muscle, fibroblast, immune cells, hepatic, splenic, lung, circulating blood cells, reproductive cells, gastrointestinal, renal, bone marrow, and pancreatic cells. The cell can be a cell line, a stem cell, or a primary cell isolated from any tissue including, but not limited to brain, liver, lung, gut, stomach, fat, muscle, testes, uterus, ovary, skin, spleen, endocrine organ and bone, etc. Where the cell is maintained in vitro, conventional tissue culture conditions and methods can be used, and are known to those of skill in the art. Isolation and culture methods for various cells are well within the knowledge of one skilled in the art. The cell can be a prokaryotic cell, a eukaryotic cell, a mammalian cell or a human cell. In one embodiment, the cell is a neuron or other cell of the brain. In some embodiments, the cell is cardiomyocyte that has been differentiated from an induced pluripotent cell.

In some embodiment of the invention, multiple cells types may be incorporated into an assay. The present invention, in some embodiments, may not be limited to one type of cell. Instead, multiple types of cells may be tested in parallel under the same or different conditions. Similarly, multiple types of cells may be tested and interconnected. Cells from different organs may be co-cultured or assayed and interconnected to determine critical drug interactions in multiple cell and tissue types.

### 1. Obtaining Cells

Methods include obtaining cells and converting them to excitable cells. Cells that are useful according to the invention include eukaryotic and prokaryotic cells. Eukaryotic cells include cells of non-mammalian invertebrates, such as yeast, plants, and nematodes, as well as non-mammalian vertebrates, such as fish and birds. The cells also include mammalian cells, including mouse, rat, and human cells. The cells also include immortalized cell lines such as HEK, HeLa, CHO, 3T3, PC12, which may be particularly useful in applications of the methods for drug screens. The cells also include stem cells, embryonic stem cells, pluripotent cells, progenitor cells, and induced pluripotent cells. Differentiated cells including cells differentiated from the stem cells, pluripotent cells and progenitor cells are included as well.

Cells are obtained by any suitable means. For example, methods of the invention can include obtaining one or more cells such as fibroblasts from an organism such as a person or animal. In some embodiments, a dermal biopsy is performed to obtain dermal fibroblasts. The skin is anesthetized and a sterile 3 mm punch is used to apply pressure and make a drilling motion until the punch has pierced the epidermis. A biopsy sample is lifted out and transferred to a sterile BME fibroblast medium after optional washing with PBS and evaporation of the PBS. The biopsy site on the patient is dressed (e.g., with an adhesive bandage). Suitable methods and devices for obtaining the cells are discussed in U.S. Pat. No. 8,603,809; U.S. Pat. No. 8,403,160; U.S. Pat. No. 5,591,444; U.S. Pub. 2012/0264623; and U.S. Pub. 2012/0214236, the contents of each of which are incorporated by reference. Any tissue culture technique that is suitable for the obtaining and propagating biopsy specimens may be used such as those discussed in Freshney, Ed., 1986, Animal Cell Culture: A Practical Approach, IRL Press, Oxford England; and Freshney, Ed., 1987, Culture of Animal Cells: A Manual of Basic Techniques, Alan R. Liss & Co., New York, both incorporated by reference.

Obtained cells may be converted into any electrically excitable cells such as neurons, specific neuronal subtypes, astrocytes or other glia, cardiomyocytes, or immune cells. Additionally, cells may be converted and grown into co-cultures of multiple cell types (e.g. neurons+glia, neurons+cardiomyocytes, neurons+immune cells).

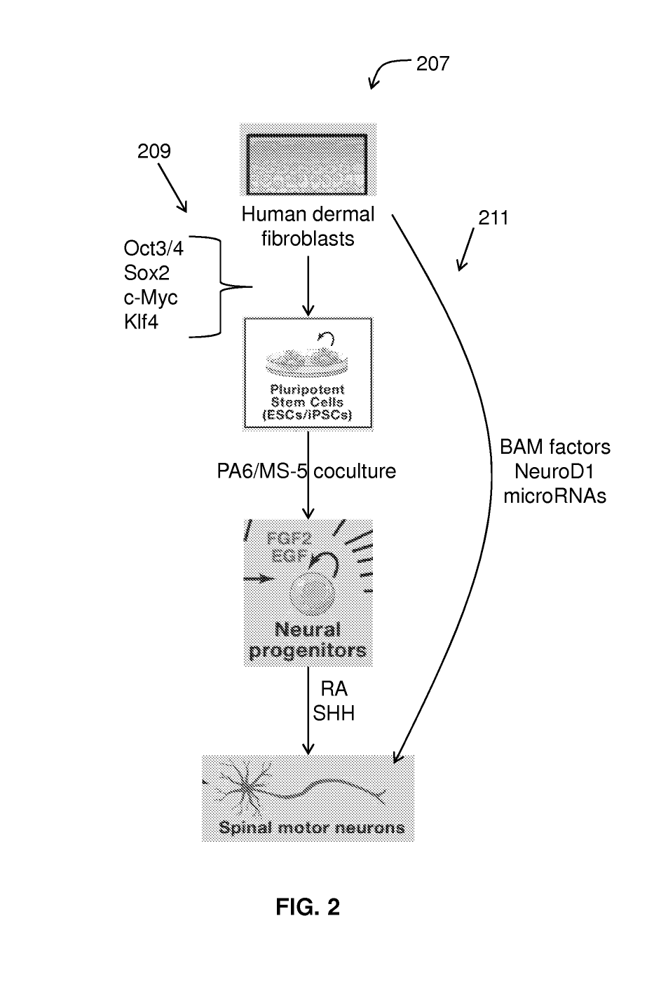

FIG. 2 illustrates exemplary pathways for converting cells into specific neural subtypes. A cell may be converted to a specific neural subtype (e.g., motor neuron). Suitable methods and pathways for the conversion of cells include pathway 209, conversion from somatic cells to induced pluripotent stem cells (iPSCs) and conversion of iPSCs to specific cell types, or pathways 211 direct conversion of cells in specific cell types.

Following pathways 209, somatic cells may be reprogrammed into induced pluripotent stem cells (iPSCs) using known methods such as the use of defined transcription factors. The iPSCs are characterized by their ability to proliferate indefinitely in culture while preserving their developmental potential to differentiate into derivatives of all three embryonic germ layers. In certain embodiments, fibroblasts are converted to iPSC by methods such as those discussed in Takahashi and Yamanaka, 2006, Induction of pluripotent stem cells from mouse embryonic and adult fibroblast cultures by defined factors Cell 126:663-676; and Takahashi, et al., 2007, Induction of pluripotent stem cells from adult human fibroblasts by defined factors, Cell 131:861-872.

Induction of pluripotent stem cells from adult fibroblasts can be done by methods that include introducing four factors, Oct3/4, Sox2, c-Myc, and Klf4, under ES cell culture conditions. Human dermal fibroblasts (HDF) are obtained. A retroviruses containing human Oct3/4, Sox2, Klf4, and c-Myc is introduced into the HDF. Six days after transduction, the cells are harvested by trypsinization and plated onto mitomycin C-treated SNL feeder cells. See, e.g., McMahon and Bradley, 1990, Cell 62:1073-1085. About one day later, the medium (DMEM containing 10% FBS) is replaced with a primate ES cell culture medium supplemented with 4 ng/mL basic fibroblast growth factor (bFGF). See Takahashi, et al., 2007, Cell 131:861. Later, hES cell-like colonies are picked and mechanically disaggregated into small clumps without enzymatic digestion. Each cell should exhibit morphology similar to that of human ES cells, characterized by large nuclei and scant cytoplasm. The cells after transduction of HDF are human iPS cells. DNA fingerprinting, sequencing, or other such assays may be performed to verify that the iPS cell lines are genetically matched to the donor.

These iPS cells can then be differentiated into specific neuronal subtypes. Pluripotent cells such as iPS cells are by definition capable of differentiating into cell types characteristic of different embryonic germ layers. A property of both embryonic stem cells human iPS cells is their ability, when plated in suspension culture, to form embryoid bodies (EBs). EBs formed from iPS cells are treated with two small molecules: an agonist of the sonic hedgehog (SHH) signaling pathway and retinoic acid (RA). For more detail, see the methods described in Dimos et al., 2008, Induced pluripotent stem cells generated from patients with ALS can be differentiated into motor neurons, Science 321(5893):1218-21; Amoroso et al., 2013, Accelerated high-yield generation of limb-innervating motor neurons from human stem cells, J Neurosci 33(2):574-86; and Boulting et al., 2011, A functionally characterized test set of human induced pluripotent stem cells, Nat Biotech 29(3):279-286; Davis-Dusenbery et al., 2014, How to make spinal motor neurons, Development 141(3):491-501; Sandoe and Eggan, 2013, Opportunities and challenges of pluripotent stem cell neurodegenerative disease models, Nat Neuroscience 16(7):780-9; and Han et al., 2011, Constructing and deconstructing stem cell models of neurological disease, Neuron 70(4):626-44.

By pathway 211, human somatic cells are obtained and direct lineage conversion of the somatic cells into motor neurons may be performed.

2. Converting Cells into Neurons, Cardiomyocytes, and Neural Sub-Types

Obtained cells may be converted into any electrically excitable cells such as neurons, specific neuronal subtypes, astrocytes or other glia, cardiomyocytes, or immune cells. Additionally, cells may be converted and grown into co-cultures of multiple cell types (e.g. neurons+glia, neurons+cardiomyocytes, neurons+immune cells).

FIG. 2 illustrates exemplary pathways for converting cells into specific neural subtypes. A cell may be converted to a specific neural subtype (e.g., motor neuron). Suitable methods and pathways for the conversion of cells include pathway 209, conversion from somatic cells to induced pluripotent stem cells (iPSCs) and conversion of iPSCs to specific cell types, or pathways 211 direct conversion of cells in specific cell types.

2a. Conversion of Cells to iPSs and Conversion of iPSs to Specific Cell Types

Following pathways 209, somatic cells may be reprogrammed into induced pluripotent stem cells (iPSCs) using known methods such as the use of defined transcription factors. The iPSCs are characterized by their ability to proliferate indefinitely in culture while preserving their developmental potential to differentiate into derivatives of all three embryonic germ layers. In certain embodiments, fibroblasts are converted to iPSC by methods such as those discussed in Takahashi and Yamanaka, 2006, Induction of pluripotent stem cells from mouse embryonic and adult fibroblast cultures by defined factors Cell 126:663-676; and Takahashi, et al., 2007, Induction of pluripotent stem cells from adult human fibroblasts by defined factors, Cell 131:861-872.

Induction of pluripotent stem cells from adult fibroblasts can be done by methods that include introducing four factors, Oct3/4, Sox2, c-Myc, and Klf4, under ES cell culture conditions. Human dermal fibroblasts (HDF) are obtained. A retroviruses containing human Oct3/4, Sox2, Klf4, and c-Myc is introduced into the HDF. Six days after transduction, the cells are harvested by trypsinization and plated onto mitomycin C-treated SNL feeder cells. See, e.g., McMahon and Bradley, 1990, Cell 62:1073-1085. About one day later, the medium (DMEM containing 10% FBS) is replaced with a primate ES cell culture medium supplemented with 4 ng/mL basic fibroblast growth factor (bFGF). See Takahashi, et al., 2007, Cell 131:861. Later, hES cell-like colonies are picked and mechanically disaggregated into small clumps without enzymatic digestion. Each cell should exhibit morphology similar to that of human ES cells, characterized by large nuclei and scant cytoplasm. The cells after transduction of HDF are human iPS cells. DNA fingerprinting, sequencing, or other such assays may be performed to verify that the iPS cell lines are genetically matched to the donor.

These iPS cells can then be differentiated into specific neuronal subtypes. Pluripotent cells such as iPS cells are by definition capable of differentiating into cell types characteristic of different embryonic germ layers. A property of both embryonic stem cells human iPS cells is their ability, when plated in suspension culture, to form embryoid bodies (EBs). EBs formed from iPS cells are treated with two small molecules: an agonist of the sonic hedgehog (SHH) signaling pathway and retinoic acid (RA). For more detail, see the methods described in Dimos et al., 2008, Induced pluripotent stem cells generated from patients with ALS can be differentiated into motor neurons, Science 321(5893):1218-21; Amoroso et al., 2013, Accelerated high-yield generation of limb-innervating motor neurons from human stem cells, J Neurosci 33(2):574-86; and Boulting et al., 2011, A functionally characterized test set of human induced pluripotent stem cells, Nat Biotech 29(3):279-286; Davis-Dusenbery et al., 2014, How to make spinal motor neurons, Development 141(3):491-501; Sandoe and Eggan, 2013, Opportunities and challenges of pluripotent stem cell neurodegenerative disease models, Nat Neuroscience 16(7):780-9; and Han et al., 2011, Constructing and deconstructing stem cell models of neurological disease, Neuron 70(4):626-44.

2b. Direct Conversion of Cells in Specific Cell Types

By pathway 211, human somatic cells are obtained and direct lineage conversion of the somatic cells into motor neurons may be performed. Conversion may include the use of lineage-specific transcription factors to induce the conversion of specific cell types from unrelated somatic cells. See, e.g., Davis-Dusenbery et al., 2014, How to make spinal motor neurons, Development 141:491; Graf, 2011, Historical origins of transdifferentiation and reprogramming, Cell Stem Cell 9:504-516. It has been shown that a set of neural lineage-specific transcription factors, or BAM factors, causes the conversion of fibroblasts into induced neuronal(iN) cells. Vierbuchen 2010 Nature 463:1035. MicroRNAs and additional pro-neuronal factors, including NeuroD1, may cooperate with or replace the BAM factors during conversion of human fibroblasts into neurons. See, for example, Ambasudhan et al., 2011, Direct reprogramming of adult human fibroblasts to functional neurons under defined conditions, Cell Stem Cell 9:113-118; Pang et al., 2011, Induction of human neuronal cells by defined transcription factors, Nature 476:220-223; also see Yoo et al., 2011, MicroRNA mediated conversion of human fibroblasts to neurons, Nature 476:228-231.

2c. Maintenance of Differentiated Cells

Differentiated cells such as motor neurons may be dissociated and plated onto glass coverslips coated with poly-d-lysine and laminin. Motor neurons may be fed with a suitable medium such as a neurobasal medium supplemented with N2, B27, GDNF, BDNF, and CTNF. Cells may be maintained in a suitable medium such as an N2 medium (DMEM/F12 [1:1] supplemented with laminin [1 μg/mL; Invitrogen], FGF-2 [10 ng/ml; R&D Systems, Minneapolis, Minn.], and N2 supplement [1%; Invitrogen]), further supplemented with GDNF, BDNF, and CNTF, all at 10 ng/ml. Suitable media are described in Son et al., 2011, Conversion of mouse and human fibroblasts into functional spinal motor neurons, Cell Stem Cell 9:205-218; Vierbuchen et al., 2010, Direct conversion of fibroblasts to functional neurons by defined factors, Nature4 63:1035-1041; Kuo et al., 2003, Differentiation of monkey embryonic stem cells into neural lineages, Biology of Reproduction 68:1727-1735; and Wernig et al., 2002, Tau EGFP embryonic stem cells: an efficient tool for neuronal lineage selection and transplantation. J Neuroscience Res 69:918-24, each incorporated by reference.

### 3. Control Cell Line or Signature or Reference Value

Methods of the invention may include obtaining or observing a signal from the cell and comparing the observed signal to an expected signal, such as a signal obtained from a reference.

The term “reference” as used herein refers to a baseline value of any kind that one skilled in the art can use in the methods. In some embodiments, the reference is a cell that has not been exposed to a stimulus capable of or suspected to be capable of changing membrane potential. In one embodiment, the reference is the same cell transfected with the microbial rhodopsin but observed at a different time point. In another embodiment, the reference is the fluorescence of a homologue of Green Fluorescent Protein (GFP) operably fused to the microbial rhodopsin.

The reference signature may be obtained by obtaining a control cell that is also of the specific neural subtype and is genetically and phenotypically similar to the test cells. In certain embodiments—where, for example, a patient has a known mutation or allele at a certain locus—genetic editing is performed to correct the mutation and generate a control cell.

Genetic or genome editing techniques may proceed by any suitable method such as zinc-finger domain methods, transcription activator-like effector nucleases (TALENs), or clustered regularly interspaced short palindromic repeat (CRISPR) nucleases. Genome editing may be used to create a control cell that is isogenic but-for a variant of interest or to obtain other variants of the original genome, such as knocking out a gene, introducing a premature stop codon, interfering with a promoter region, or changing the function of an ion channel or other cellular protein. In certain embodiments, genome editing techniques are applied to the iPS cells. Genomic editing may be performed by any suitable method known in the art such as TALENs or CRISPR technology. TALENs and CRISPR methods provide one-to-one relationship to the target sites, i.e. one unit of the tandem repeat in the TALE domain recognizes one nucleotide in the target site, and the crRNA or gRNA of CRISPR/Cas system hybridizes to the complementary sequence in the DNA target. Methods can include using a pair of TALENs or a Cas9 protein with one gRNA to generate double-strand breaks in the target. The breaks are then repaired via non-homologous end-joining or homologous recombination (HR).

TALENs uses a nonspecific DNA-cleaving nuclease fused to a DNA-binding domain that can be to target essentially any sequence. For TALEN technology, target sites are identified and expression vectors are made. See Liu et al, 2012, Efficient and specific modifications of the Drosophila genome by means of an easy TALEN strategy, J. Genet. Genomics 39:209-215. The linearized expression vectors (e.g., by Not1) and used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55.

CRISPR methodologies employ a nuclease, CRISPR-associated (Cas9), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas9 and guide RNA (gRNA) may be synthesized by known methods. Cas9/guide-RNA (gRNA) uses a non-specific DNA cleavage protein Cas9, and an RNA oligo to hybridize to target and recruit the Cas9/gRNA complex. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11.

In certain embodiments, genome editing is performed using zinc finger nuclease-mediated process as described, for example, in U.S. Pub. 2011/0023144 to Weinstein.

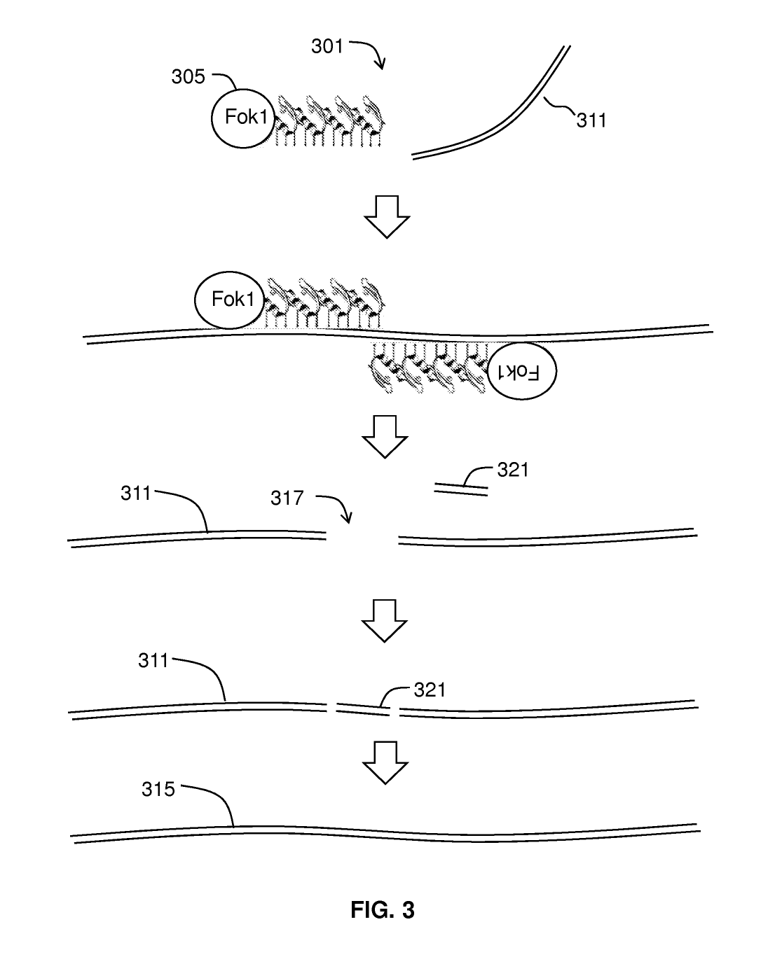

FIG. 3 gives an overview of a method 301 for zing-finger nuclease mediated editing. Briefly, the method includes introducing into the iPS cell at least one RNA molecule encoding a targeted zinc finger nuclease 305 and, optionally, at least one accessory polynucleotide. The cell includes target sequence 311. The cell is incubated to allow expression of the zinc finger nuclease 305, wherein a double-stranded break 317 is introduced into the targeted chromosomal sequence 311 by the zinc finger nuclease 305. In some embodiments, a donor polynucleotide or exchange polynucleotide 321 is introduced. Target DNA 311 along with exchange polynucleotide 321 may be repaired by an error-prone non-homologous end-joining DNA repair process or a homology-directed DNA repair process. This may be used to produce a control line with a control genome 315 that is isogenic to original genome 311 but for a changed site. See U.S. Pat. Nos. 6,607,882; 6,534,261 and 6,453,242; U.S. Pat. No. 5,789,538; U.S. Pat. No. 5,925,523; U.S. Pat. No. 6,007,988; U.S. Pat. No. 6,013,453; U.S. Pat. No. 6,410,248; U.S. Pat. No. 6,140,466; U.S. Pat. No. 6,200,759; and U.S. Pat. No. 6,242,568, each of which is incorporated by reference.

Using genome editing for modifying a chromosomal sequence, a control cell or cell line can be generated, or any other genetic variant of the first cell may be created. In certain embodiments, control cells are obtained from healthy individuals, i.e., without using genome editing on cells taken from the subject. The control line can be used to generate a control signature, or reference, for comparison to test data. In some embodiments, a control signature is stored on-file after having been previously generated and stored and the stored control signature is used (e.g., a digital file such as a graph or series of measurements stored in a non-transitory memory in a computer system). For example, a control signature could be generated by assaying a large population of subjects of known phenotype or genotype and storing an aggregate result as a control signature for later downstream comparisons.

### 4. Optogenetic Systems

In a preferred embodiment, methods of the invention include characterizing a cell by incorporating into a cell an optical actuator of electrical activity and an optical reporter of electrical activity—i.e., both into one cell or each of a plurality of cells. In some embodiments, a cell will receive one of the actuator and reporter. In certain embodiments, a cell will receive both via transfection with a single vector that includes genes coding for each of the reporter and actuator. As used herein the term “optical reporter” refers to a structure or system employed to yield an optical signal indicative of cellular electrical or neural activity such as a voltage drop across a membrane, a synaptic transmission, an action potential, a release or uptake or non-uptake of a neurotransmitter, etc. As used herein, the term “membrane potential” refers to a calculated difference in voltage between the interior and exterior of a cell. In one embodiment membrane potential, ΔV, is determined by the equation ΔV=V(interior)−V(exterior). By convention, V(exterior) is regarded as 0 V, so then ΔV=V(interior).

4a. Optogenetic Reporters

The cell and the optional control line may be caused to express an optical reporter of neural or electrical activity. Examples of neural activity include action potentials in a neuron or fusion of vesicles releasing neurotransmitters. Exemplary electrical activity includes action potentials in a neuron, cardiomyocyte, astrocyte or other electrically active cell. Further examples of neural or electrical activity include ion pumping or release or changing ionic gradients across membranes. Causing a cell to express an optical reporter of neural activity can be done with a fluorescent reporter of vesicle fusion. Expressing an optical reporter of neural or electrical activity can include transformation with an optogenetic reporter. For example, the cell may be transformed with a vector comprising an optogenetic reporter and the cell may also be caused to express an optogenetic actuator by transformation. In certain embodiments, the differentiated neurons are cultured (e.g., for about 4 days) and then infected with lentivirus bearing a genetically encoded optical reporter of neural or electrical activity and optionally an optical voltage actuator.

Any suitable optical reporter of neural or electrical activity may be used. Exemplary reporters include fluorescent reporters of transmembrane voltage differences, pHluorin-based reporters of synaptic vesicle fusion, and genetically encoded calcium indicators. In a preferred embodiment, a genetically encoded voltage indicator is used. Genetically encoded voltage indicators that may be used or modified for use with methods of the invention include FlaSh (Siegel, 1997, A genetically encoded optical probe of membrane voltage. Neuron 19:735-741); SPARC (Ataka, 2002, A genetically targetable fluorescent probe of channel gating with rapid kinetics, Biophys J 82:509-516); and VSFP1 (Sakai et al., 2001, Design and characterization of a DNA encoded, voltage-sensitive fluorescent protein, Euro J Neuroscience 13:2314-2318). A genetically encoded voltage indicator based on the paddle domain of a voltage-gated phosphatase is CiVSP (Murata et al., 2005, Phosphoinositide phosphatase activity coupled to an intrinsic voltage sensor, Nature 435:1239-1243). Another indicator is the hybrid hVOS indicator (Chanda et al., 2005, A hybrid approach to measuring electrical activity in genetically specified neurons, Nat Neuroscience 8:1619-1626), which transduces the voltage dependent migration of Dipicrylamine (DPA) through the membrane leaflet to “dark FRET” (fluorescence resonance energy transfer) with a membrane-targeted GFP. Methods of the invention may use a genetically encoded voltage indicator in which a fluorescent moiety is inserted in the voltage sensing domain. For example, in Accelerated Sensor of Action Potentials 1 (ASAP1), a circularly permuted green fluorescent protein is inserted in an extracellular loop of a voltage-sensing domain, rendering fluorescence responsive to membrane potential. In some embodiments, ASAP1 is used as a reporter. ASAP1 is described in St-Pierre et al., 2014, High-fidelity optical reporting of neuronal electrical activity with an ultrafast fluorescent voltage sensor, Nature Neuroscience 17(6):884-889.

Any suitable voltage reporter may be included such as, for example, Arch variants, one of the QuasArs, an electrochromic FRET (eFRET) sensor comprising a fluorescent protein fused to an Arch mutant, or any of the other ones disclosed or discussed herein. See Peng et al., 2014, Bright and fast multicoloured voltage reporters via electrochromic FRET, Nat Comm 5, incorporated by reference. In the eFRET variants, one can use fluorescent proteins of a variety of colors fused to an Arch mutant, typical a QuasAr, to produce a bright and fast fluorescent voltage sensor. It may be suitable to use a green flourescent protein (GFP) or the variant known as enhanced GFP (eGFP). The eGFP features an excitation spectral profile that overlays nicely with the 488 nm argon-ion laser line and is similar in profile to fluorescein and related synthetic fluorophores that are readily imaged using commonly available filter sets designed for fluorescein (FITC). Furthermore, EGFP is among the brightest and most photostable of the Aequorea-based voltage reporters.

A yellow flourescent protein (YFP) with a Q69M mutation is dubbed citrine. The mutation increases the acid stability of citrine, while simultaneously reducing its chloride sensitivity. In addition, Citrine is expressed more efficiently in mammalian cell culture (especially when targeted to acidic organelles) and is more photostable than many previous yellow fluorescent proteins. Citrine features absorption and fluorescence emission maxima at 516 and 529 nm, respectively, and is 75% brighter than EGFP.

A directed evolution approach of mRFP1, targeting certain amino acid residues followed by selecting for new color variants, resulted in a group of six monomeric FPs exhibiting emission maxima ranging from 540 nm to 610 nm. Those voltage reporters are named mHoneydew, mBanana, mOrange, mTangerine, mStrawberry, and mCherry (the “m” referring to monomer). The mOrange reporter is the brightest of the mFruit proteins and has spectral characteristics allowing it to be paired with other voltage reporters in the cyan and green spectral region for multicolor imaging and as a potential FRET acceptor. For additional discussion, see Day and Davidson, 2009, The fluorescent protein palette, Chem Soc Rev 38(10):2887-2921; Shaner et al., 2005, A guide to choosing fluorescent proteins, Nat Methods 2(12):905-909; Shaner et al., 2008, Improving the photostability of bright monomeric orange and red fluorescent proteins, Nat Meth 5(6):545-551; U.S. Pat. No. 6,066,476; U.S. Pat. No. 6,469,154; and U.S. Pat. No. 7,060,793, the contents of each of which are incorporated by reference entirely for all purposes.

In certain embodiments, an optical reporter of electrical activity in a cell is provided by a microbial rhodopsin or a modified microbial rhodopsin. A typical microbial rhodopsin is a light-driven proton pump structured as an integral membrane protein belonging to the family of Archaeal rhodopsins. Archaeal rhodopsins are characterized by seven transmembrane helices with a retinal chromophore buried therein, the retinal chromophore being covalently bound to conserved lysine residue in one of the helices via a Schiff base. See Neutze et al., 2002, Bacteriorhodopsin: a high-resolution structural view of vectorial proton transport, Biochimica et Biophysica Acta 1565:144-167; Beja et al., 2001, Proteorhodopsin phototrophy in the ocean, Nature 411:786-789. The invention includes the insight that microbial rhodopsins or modified microbial rhodopsins that have reduced ion pumping activity—compared to the natural microbial rhodopsin protein from which they are derived—can be used as an optically detectable sensor to sense voltage across membranous structures, such as in cells and sub-cellular organelles when they are present in the lipid bilayer membrane. That is, the microbial rhodopsin proteins and the modified microbial rhodopsin proteins can be used as optical reporters to measure changes in membrane potential of a cell, including prokaryotic and eukaryotic cells. The optical reporters described herein are not constrained by the need for electrodes and permit electrophysiological studies to be performed in e.g., subcellular compartments (e.g., mitochondria) or in small cells (e.g., bacteria). The optical reporters described herein can be used in methods for drug screening, in research settings, and in in vivo imaging systems.

The retinal chromophore imbues microbial rhodopsins with unusual optical properties. The linear and nonlinear responses of the retinal are highly sensitive to interactions with the protein host: small changes in the electrostatic environment can lead to large changes in absorption spectrum. These electro-optical couplings provide the basis for voltage sensitivity in microbial rhodopsins.

Some of the optical reporters described herein are natural proteins without modifications and are used in cells that do not normally express the microbial rhodopsin transfected to the cell, such as eukaryotic cells. For example, as shown in the examples, the wild type Archaerhodopsin 3 can be used in neural cells to specifically detect membrane potential and changes thereto.

Some of the microbial rhodopsins are derived from a microbial rhodopsin protein by modification of the protein to reduce or inhibit light-induced ion pumping of the rhodopsin protein. Such modifications permit the modified microbial rhodopsin proteins to sense voltage without altering the membrane potential of the cell with its native ion pumping activity. Other mutations impart other advantageous properties to microbial rhodopsin voltage sensors, including increased fluorescence brightness, improved photostability, tuning of the sensitivity and dynamic range of the voltage response, increased response speed, and tuning of the absorption and emission spectra.

Provided herein are illustrative exemplary optical voltage reporters and directions for making and using such sensors. Other sensors that work in a similar manner as optical reporters can be prepared and used based on the description and the examples provided herein.

Exemplary microbial rhodopsins include: green-absorbing proteorhodopsin (GPR, Gen Bank #AF349983), a light-driven proton pump found in marine bacteria; blue absorbing proteorhodopsin (BPR, GenBank #AF349981), a light-driven proton pump found in marine bacteria; Natronomonas pharaonis sensory rhodopsin II (NpSRII, GenBank #Z35086.1), a light-activated signaling protein found in the halophilic bacterium N. pharaonis; bacteriorhodopsin (BR, GenBank #NC_010364.1), a light-driven proton pump found in Halobacterium salinarum; Archaerhodopsin 3 (Arch3, GenBank #P96787), a light-driven proton pump found in Halobacterium sodomense; variants of the foregoing; and others discussed herein. Additional rhodopsions that can be mutated as indicated in the methods of the invention include fungal opsin related protein (Mac, GenBank #AAG01180); Cruxrhodopsin (Crux, GenBank #BAA06678); Algal bacteriorhodopsin (Ace, GenBank #AAY82897); Archaerhodopsin 1 (Arch 1, GenBank #P69051); Archaerhodopsin 2 (Arch 2, GenBank #P29563); and Archaerhodopsin 4 (Arch 4, GenBank #AAG42454). Some of the foregoing are pointed to by GenBank number. However, a rhodopsin may vary from a sequence in GenBank. Based on the description of the motif described herein, a skilled artisan will easily be able to make homologous mutations in microbial rhodopsin genes to achieve the described or desired functions, e.g. reduction in the pumping activity of the microbial rhodopsin in question.

In one embodiment, the green-absorbing proteorhodopsin (GPR) is used as a starting molecule to provide an optical reporter. This molecule is selected for its relatively red-shifted absorption spectrum and its ease of expression in heterologous hosts such as E. coli. In another embodiment, the blue-absorbing proteorhodopsin (BPR) is used as an optical reporter of voltage. Microbial rhodopsins are sensitive to quantities other than voltage. Mutants of GPR and BPR, as described herein, are also sensitive to intracellular pH. It is also contemplated that mutants of halorhodopsin may be sensitive to local chloride concentration. GPR has seven spectroscopically distinguishable states that it passes through in its photocycle. In principle the transition between any pair of states is sensitive to membrane potential. In one embodiment, the acid-base equilibrium of the Schiff base is chosen as the wavelength-shifting transition, hence the name of the reporter: Proteorhodopsin Optical Proton Sensor (PROPS). The absorption spectrum of wild-type GPR is known to depend sensitively on the state of protonation of the Schiff base. When protonated, the absorption maximum is at 545 nm, and when deprotonated the maximum is at 412 nm. When GPR absorbs a photon, the retinal undergoes a 13-trans to cis isomerization, which causes a proton to hop from the Schiff base to nearby Asp97, leading to a shift from absorption at 545 nm to 412 nm. The PROPS design described herein seeks to recapitulate this shift in response to a change in membrane potential.

Two aspects of wild-type GPR can be changed for it to serve as an optimal voltage sensor. First, the pKa of the Schiff base can be shifted from its wild-type value of 12 to a value close to the ambient pH. When pKa approximately equals pH, the state of protonation becomes maximally sensitive to the membrane potential. Second, the endogenous charge-pumping capability can be eliminated so the reporter does not perturb the quantity under study. However, in some situations, a wild type microbial rhodopsin can be used, such as Arch 3 WT, which functions in neurons to measure membrane potential as shown in our examples.

In one embodiment, a single point mutation induces both changes in GPR. Mutating Asp97 to Asn eliminates a negative charge near the Schiff base, and destabilizes the proton on the Schiff base. The pKa shifts from about 12 to 9.8. In wild-type GPR, Asp97 also serves as the proton acceptor in the first step of the photocycle, so removing this amino acid eliminates proton pumping. This mutant of GPR is referred to herein as PROPS. Similarly, in an analogous voltage sensor derived from BPR, the homologous mutation Asp99 to Asn lowers the pKa of the Schiff base and eliminates the proton-pumping photocycle. Thus, in one embodiment the optical reporter is derived from BPR in which the amino acid residue Asp99 is mutated to Asn.

In GPR, additional mutations shift the pKa closer to the physiological value of 7.4. In particular, mutations Glu108 to Gln and Glu142 to Gln individually or in combination lead to decreases in the pKa and to further increases in the sensitivity to voltage. Many mutations other than those discussed herein may lead to additional changes in the pKa and improvements in the optical properties of PROPS and are contemplated herein.

The invention provides reporters based on rhodop sins with introduced mutations. For example, mutations that eliminate pumping in microbial rhodopsins in the present invention generally comprise mutations to the Schiff base counterion; a carboxylic amino acid (Asp or Glu) conserved on the third transmembrane helix (helix C) of the rhodopsin proteins. Mutations to the carboxylic residue directly affect the proton conduction pathway, eliminating proton pumping (e.g., Asp to Asn, Gln, or His mutation, or Glu to Asn Gln, or His mutation). Mutating the proton acceptor aspartic acid adjacent the Schiff base to asparagine suppresses proton pumping. Thus, in some embodiments, the mutations are selected from the group consisting of: D97N (green-absorbing proteorhodopsin), D95N (Archaerhodopsin 3), D99N (blue-absorbing proteorhodopsin), D75N (sensory rhodopsin II), and D85N (bacteriorhodopsin). In other embodiments, residues that can be mutated to inhibit pumping include (using bacteriorhodopsin numbering) D96, Y199, and R82, and their homologues in other microbial rhodopsins. In another embodiment, residue D95 can be mutated in Archaerhodopsin to inhibit proton pumping (e.g., D95N). Residues near the binding pocket can be mutated singly or in combination to tune the spectra to a desired absorption and emission wavelength. In bacteriorhodopsin these residues include, but are not limited to, L92, W86, W182, D212, I119, and M145. Homologous residues may be mutated in other microbial rhodopsins. Thus, in some embodiments, the mutation to modify the microbial rhodopsin protein is performed at a residue selected from the group consisting of L92, W86, W182, D212, I119, M145. Mutations can shift the dynamic range of voltage sensitivity into a desired band by shifting the distribution of charge in the vicinity of the Schiff base, and thereby changing the voltage needed to add or remove a proton from this group. Voltage-shifting mutations in green-absorbing proteorhodopsin include, but are not limited to, E108Q, E142Q, L217D, either singly or in combination using green-absorbing proteorhodopsin locations as an example, or a homologous residue in another rhodopsin. In one embodiment, a D95N mutation is introduced into Archaerhodopsin 3 to adjust the pKa of the Schiff base towards a neutral pH. Additionally or alternatively, mutations can enhance brightness, photostability, or both. Residues which, when mutated, may restrict the binding pocket to increase fluorescence include (using bacteriorhodopsin numbering) Y199, Y57, P49, V213, and V48.

Optical reporters that may be suitable for use with the invention include those that use the endogenous fluorescence of the microbial rhodopsin protein Archaerhodopsin 3 (Arch) from Halorubum sodomense. Arch resolves action potentials with high signal-to-noise (SNR) and low photo-toxicity.

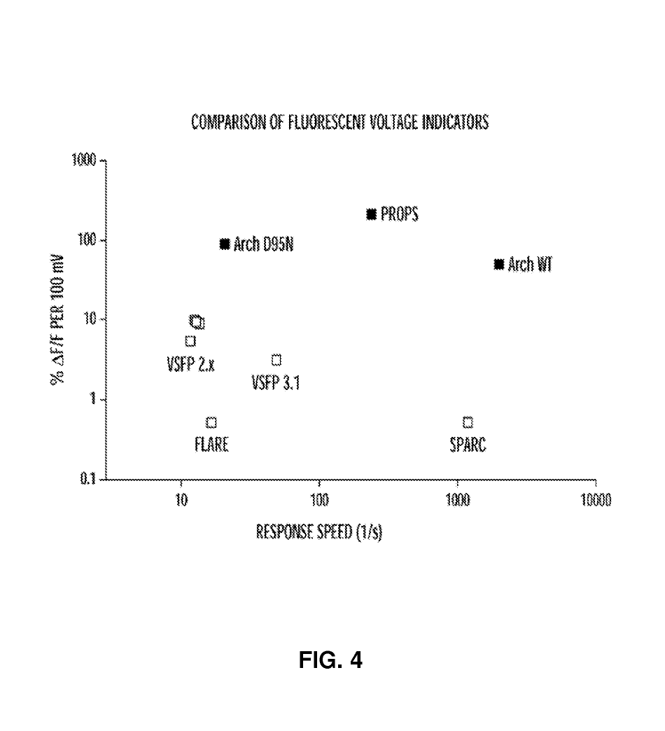

FIG. 4 shows genetically encoded fluorescent voltage indicators classified according to their sensitivity and speed-the two key parameters that determine the performance of an indicator. The invention provides reporters such as Proteorhodopsin Optical Proton Sensor (PROPS), Arch 3 WT, and Arch 3 D95N, shown on the upper right. PROPS functions in bacteria, while Arch 3 WT and Arch 3 D95N function in mammalian cells. Such microbial rhodopsin-based voltage indicators are faster and far more sensitive than other indicators.

The invention may use optical reporters that include fluorescent voltage indicating proteins such as VSFP 2.3 (Knopfel et al., 2010, J Neurosci 30:14998-15004), which exhibits a response time of 78 ms and f (where f=(delta F/F per 100 mV)) of 9.5%. VSFP 2.4 (Ibid.) has a 72 ms response time and f of 8.9%. VSFP 3.1 (Lundby et al., 2008, PLoSOne 3:2514) has a response time of 1-20 ms and a F of 3%. Mermaid is a molecule described in Perron et al., 2009, Front Mol Neurosci 2:1-8 with a response time of 76 and a F of 9.2%. SPARC (Ataka & Pieribone, 2002, Biophys J 82:509-516) response time 0.8 ms and F 0.5%. Flash (Siegel, 1997, Neuron 19:735-741) has response time 2.8-85 ms and f of 5.1%. Arch 3 WT has a response time of <0.5 ms and f of 66%. Arch D95N has a response time of 41 ms and f of 100%.

Optical recording of action potentials were made in a single rat hippocampal neuron.

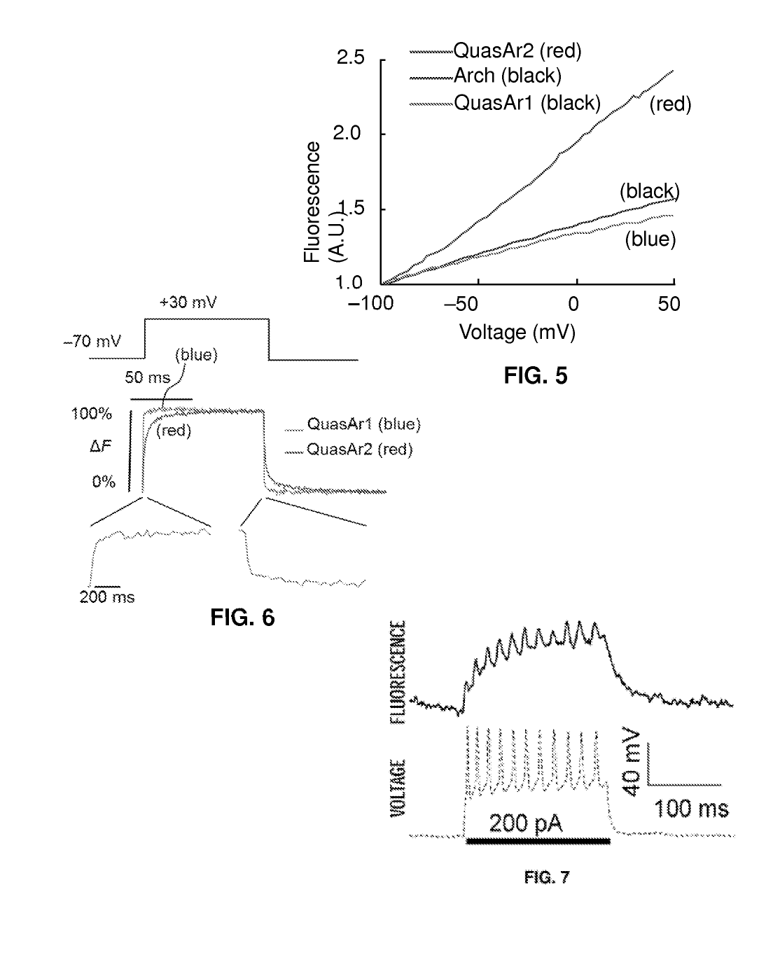

FIG. 5 shows the dependence of fluorescence on membrane voltage of Archaerhodopsin-based voltage indicators.

FIG. 6 shows electrically recorded membrane potential of a neuron expressing QuasArs.

FIG. 7 shows whole-cell membrane potential determined via electrical recording (bottom, voltage line) and weighted ArchD95N fluorescence (top, fluorescence line) during a single-trial recording of a train of action potentials. The data represents a single trial, in which spiking was induced by injection of a current pulse. The fluorescence shows clear bursts accompanying individual action potentials. This experiment is the first robust measurement of action potentials in a single mammalian neuron using a genetically encoded voltage indicator.

QuasAr2 refers to a specific variant of Arch. As discussed, archaerhodopsin 3 (Arch) functions as a fast and sensitive voltage indicator. Improved versions of Arch include the QuasArs (‘quality superior to Arch’), described in Hochbaum et al., 2014. QuasAr1 differs from wild-type Arch by the mutations P60S, T80S, D95H, D106H and F161V. QuasAr2 differed from QuasAr1 by the mutation H95Q.

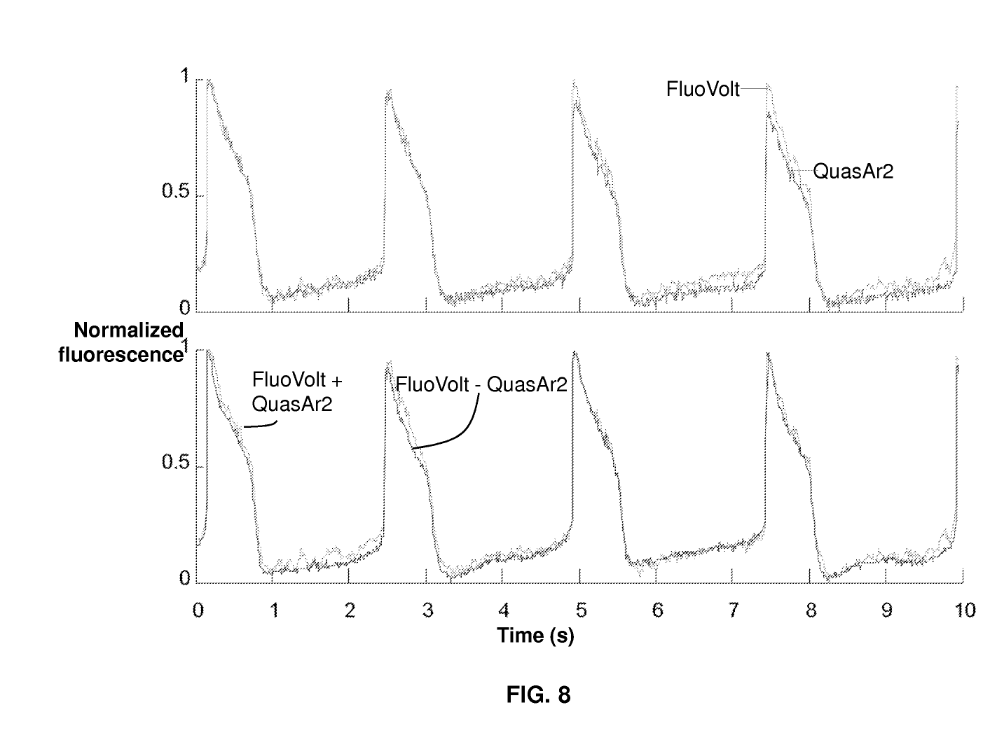

FIG. 8 compares cardiomyocyte action potential waveforms, as measured by the genetically encoded voltage indicator QuasAr2 and the voltage-sensitive dye, FluoVolt. Cells were sparsely transfected with the QuasAr2 construct and then treated with FluoVolt dye. QuasAr2 was excited by red laser light at a wavelength of 635 nm with fluorescence detection centered at 720 nm. FluoVolt was excited by 488 nm laser light with fluorescence detection centered at 525 nm. The top panel shows the simultaneously recorded AP waveforms from a cell expressing QuasAr2 (red line) and labeled with FluoVolt (green line). The similarity of these traces establishes that QuasAr2 fluorescence accurately represents the underlying AP waveform. The lower trace compares the FluoVolt AP waveform in the presence (FluoVolt+, QuasAr2+, green) and absence (FluoVolt+, QuasAr2−, cyan) of QuasAr2 expression. The similarity of these two traces establishes that expression of QuasAr2 does not perturb the AP waveform.

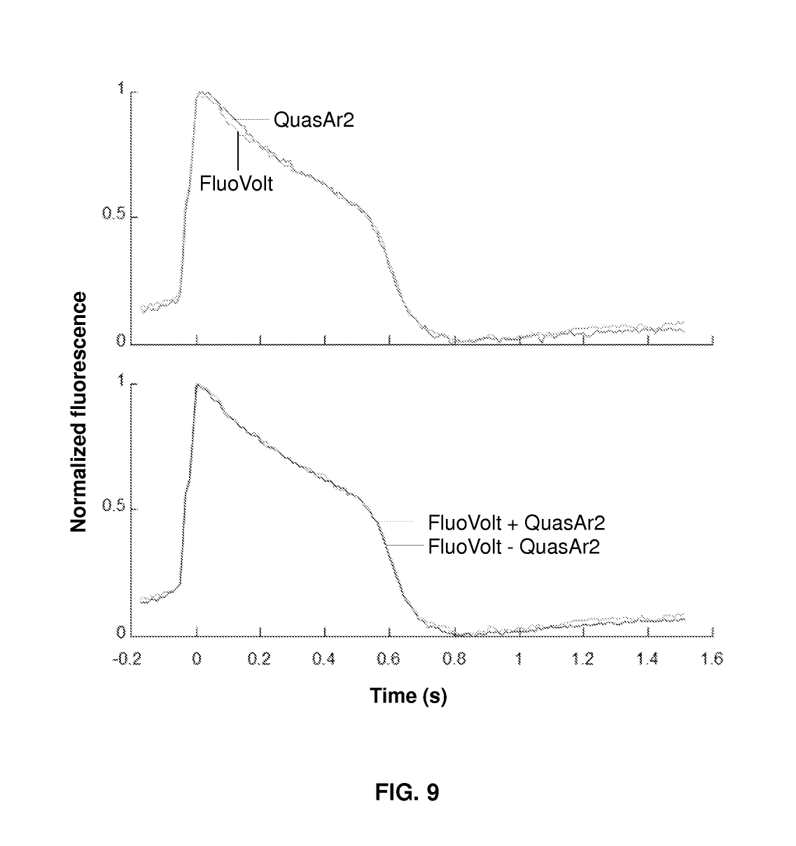

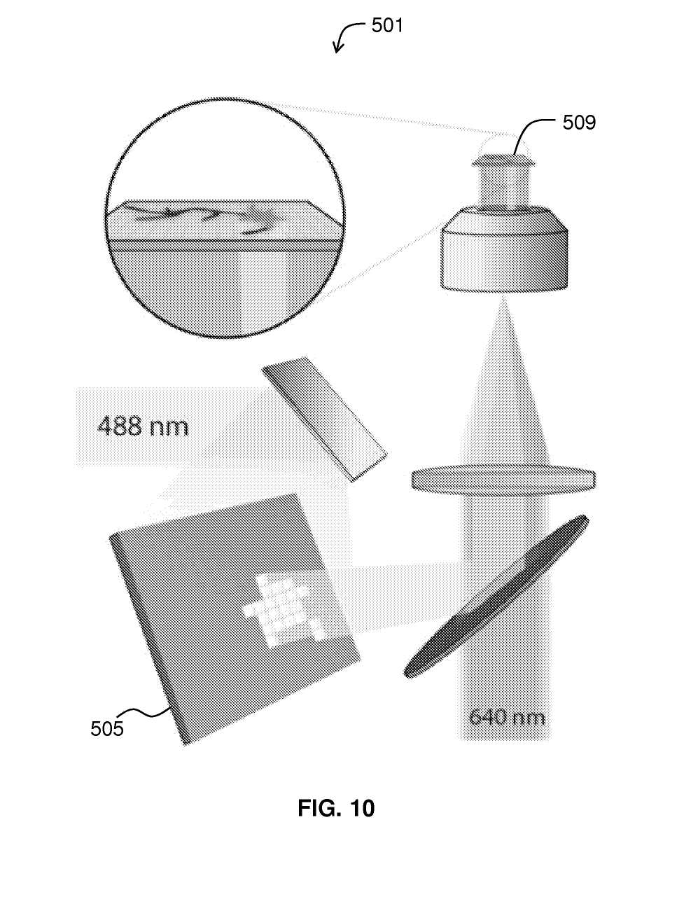

FIG. 9 shows plots of the average waveforms from the traces in FIG. 8. Arch and the above-mentioned variants target eukaryotic membranes and can image single action potentials and subthreshold depolarization in cultured mammalian neurons. See Kralj et al, 2012, Optical recording of action potentials in mammalian neurons using a microbial rhodopsin, Nat Methods 9:90-95. See Hochbaum et al., All-optical electrophysiology in mammalian neurons using engineered microbial rhodopsins, Nature Methods, published online Jun. 22, 2014. Thus Arch and variants of Arch may provide good optical reporters of neural activity according to embodiments of the invention.

The invention provides optical reporters based on Archaerhodopsins that function in mammalian cells, including human stem cell-derived neurons and cardiomyocytes. These proteins indicate electrical dynamics with sub-millisecond temporal resolution and sub-micron spatial resolution and may be used in non-contact, high-throughput, and high-content studies of electrical dynamics in cells and tissues using optical measurement of membrane potential. These reporters are broadly useful, particularly in eukaryotic, such as mammalian, including human cells.

The invention includes reporters based on Archaerhodopsin 3 (Arch 3) and its homologues. Arch 3 is Archaerhodopsin from H. sodomense and it is known as a genetically-encoded reagent for high-performance yellow/green-light neural silencing. Gene sequence at GenBank: GU045593.1 (synthetic construct Arch 3 gene, complete cds. Submitted Sep. 28, 2009). These proteins localize to the plasma membrane in eukaryotic cells and show voltage-dependent fluorescence.

Exemplary sequences that can be used to generate virus constructs with Arch 3 include a lentivirus backbone with promoters such as CamKII (excitatory neuron specific); hSynapsin (pan neuronal); CAG enhancer (pan cellular); CMV (pan cellular); Ubiquitin (pan cellular); others; or a combination thereof.

The invention may use optical reporters that include fluorescent voltage indicating proteins such as VSFP 2.3 (Knopfel et al., 2010, Toward the second generation of optogenetic tools, J Neurosci 30:14998-15004), which exhibits a response time of 78 ms and f (where f=(delta F/F per 100 mV)) of 9.5%. VSFP 2.4 (Ibid.) has a 72 ms response time and f of 8.9%. VSFP 3.1 (Lundby et al., 2008, Engineering of a genetically encodable fluorescent voltage sensor exploiting fast Ci-VSP voltage-sensing movements, PLoSOne 3:2514) has a response time of 1-20 ms and a f of 3%. Mermaid is a molecule described in Perron et al., 2009, Second and third generation voltage-sensitive fluorescent proteins for monitoring membrane potential, Front Mol Neurosci 2:1-8 with a response time of 76 and a F of 9.2%. SPARC (Ataka & Pieribone, 2002, Biophys J 82:509-516) response time 0.8 ms and F 0.5%. Flash (Siegel, 1997, Neuron 19:735-741) has response time 2.8-85 ms and f of 5.1%. Arch 3 WT has a response time of 0.6 ms and f of 32%.

Fluorescence recordings may be acquired on an epifluorescence microscope, described in Kralj et al., 2012, Optical recording of action potentials in mammalian neurons using a microbial rhodopsin, Nat. Methods 9:90-95.

Optical reporters of the invention show high sensitivity. In mammalian cells, optical reporters show about 3-fold increase in fluorescence between −150 mV and +150 mV. The response is linear over most of this range. Membrane voltage can be measured with a precision of <1 mV in a 1 s interval. Reporters of the invention show high speed. Arch 3 WT shows 90% of its step response in 0.6 ms. A neuronal action potential lasts approximately 1 ms, so the speeds of Arch indicators meet the benchmark for imaging electrical activity of neurons. Arch 3 WT retains the photo-induced proton-pumping, so illumination slightly hyperpolarizes the cell. Reporters of the invention show high photo-stability and are comparable to GFP in the number of fluorescence photons produced prior to photobleaching. The reporters may also show far red spectrum. The voltage-indicating protein reporters, sometimes referred to as genetically encoded voltage indicators (GEVIs), may be excited with a laser at wavelengths between 590-640 nm, and the emission is in the near infrared, peaked at 710 nm. The emission is farther to the red than any existing fluorescent protein. These wavelengths coincide with low cellular auto-fluorescence and good transmission through tissue. This feature makes these proteins particularly useful in optical measurements of action potentials as the spectrum facilitates imaging with high signal-to-noise ratio, as well as multi-spectral imaging in combination with other fluorescent probes.

The reporters can be targeted to specific locations or cell types including primary neuronal cultures, cardiomyocytes (HL-1 and human iPSC-derived), HEK cells, and Gram positive and Gram negative bacteria as well as to the endoplasmic reticulum, and to mitochondria. The constructs are useful also for in vivo imaging in C. elegans, zebrafish, mice, and rats. Using promoters specific to a particular cell type, time, or both, membrane potential may be imaged in any optically accessible cell type or organelle in a living organism. A reporter may include at least three elements: a promoter, a microbial rhodopsin voltage sensor, one or more targeting motifs, and an optional accessory fluorescent protein. Some non-limiting examples for each of these elements are rhodopsins are given above. Exemplary promoters include CMV, 14x UAS-E1b, HuC, ara, and lac. Exemplary targeting motifs include SS (beta-2nAChR) SS (PPL), ER export motif, TS from Kir2.1, and MS. Exemplary fluorescent proteins include Venus, EYFP, and TagRFP.

In one embodiment, at least one or more rhodopsin, promoter, targeting motif, and fluorescent protein is selected to create an optical voltage sensor with the desired properties. In some embodiments, methods and compositions for voltage sensing as described herein involves selecting: 1) a microbial rhodopsin protein, 2) one or more mutations to imbue the protein with sensitivity to voltage or to other quantities of interest and to eliminate light-driven charge pumping, 3) codon usage appropriate to the host species, 4) a promoter and targeting sequences to express the protein in cell types of interest and to target the protein to the sub-cellular structure of interest, 5) an optional fusion with a conventional fluorescent protein to provide ratiometric imaging, 6) a chromophore to insert into the microbial rhodopsin, and 7) an optical imaging scheme.

In one embodiment, the voltage sensor is selected from a microbial rhodopsin protein (wild-type or mutant) that provides a voltage-induced shift in its absorption or fluorescence. The starting sequences from which these constructs can be engineered include, but are not limited to, the rhodopsins and mutations discussed herein that can be made to the gene to enhance the performance of the protein product.

4b. Multimodal Sensing/Multiplexing

Membrane potential is only one of several mechanisms of signaling within cells. One may correlate changes in membrane potential with changes in concentration of other species, such as Ca²⁺, H⁺ (i.e. pH), Na⁺, ATP, cAMP, NADH.

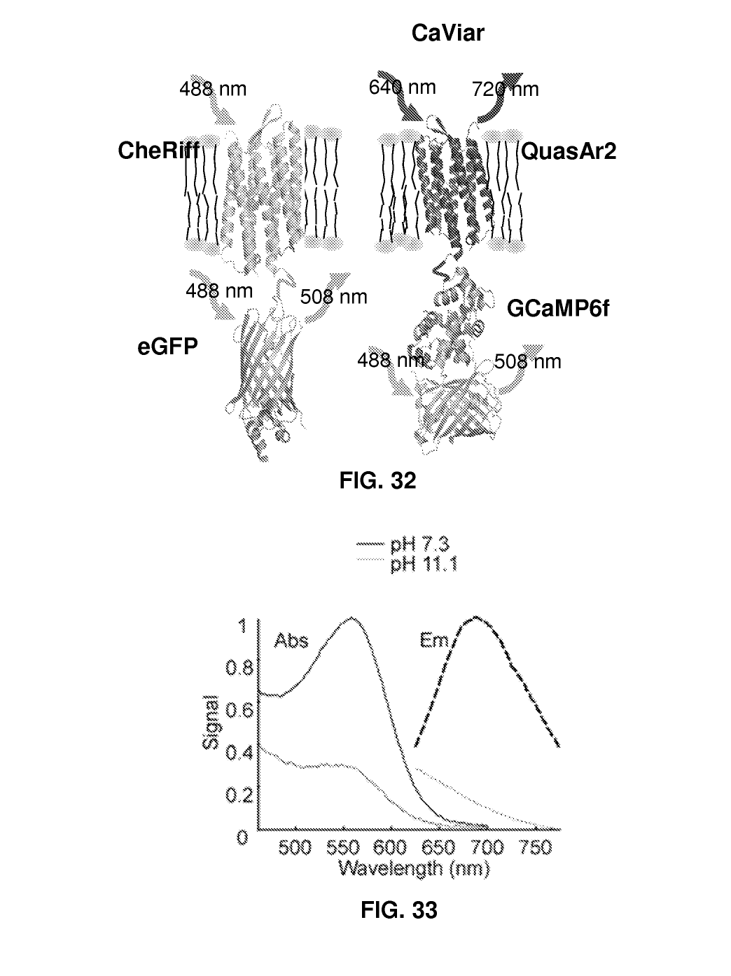

FIG. 32 shows a fusion of Arch with GCaMP6f (a fluorescent Ca²⁺ indicator). The fusion of an Arch-based voltage indicator and a genetically encoded Ca²⁺ indicator is called CaViar (See Hou, Jennifer H., et al. “Simultaneous mapping of membrane voltage and calcium in zebrafish heart in vivo reveals chamber-specific developmental transitions in ionic currents.” Frontiers in physiology 5 (2014).). One can also use fusions with other protein-based fluorescent indicators to enable other forms of multimodal imaging using the concept as taught herein. Concentration of ions such as sodium, potassium, chloride, and calcium can be simultaneously measured when the nucleic acid encoding the microbial rhodopsin is operably linked to or fused with an additional fluorescent analyte sensitive indicator; or when the microbial rhodopsin and the additional fluorescent analyte sensitive indicator are co-expressed in the same cell.

It is often desirable to achieve simultaneous optical stimulation of a cell, calcium imaging, and voltage imaging. To achieve all three modalities in the same cell, the invention provides for a violet-excited Channelrhodopsin actuator (psChR or TsChR); a red-shifted genetically encoded calcium indicator; and a far red Arch-derived voltage indicator. Red-shifted genetically encoded calcium indicators include R-GECO1 (See Zhao, Yongxin, et al. “An expanded palette of genetically encoded Ca²⁺ indicators.” Science 333.6051 (2011): 1888-1891 and Wu, Jiahui, et al. “Improved orange and red Ca²⁺ indicators and photophysical considerations for optogenetic applications.” ACS chemical neuroscience 4.6 (2013): 963-972.), R-CaMP2 (See Inoue, Masatoshi, et al. “Rational design of a high-affinity, fast, red calcium indicator R-CaMP2.” Nature methods 12.1 (2015): 64-70.), jRCaMP1a (Addgene plasmid 61562), and jRGECO1a (Addgene plasmid 61563). These calcium indicators are excited by wavelengths between 540 and 560 nm, and emit at wavelengths between 570 and 620 nm, thereby permitting spectral separation from the violet-excited channelrhodopsin actuator and the Arch-based voltage indicator.

One can combine imaging of voltage indicating proteins with other structural and functional imaging, of e.g. pH, calcium, or ATP. One may also combine imaging of voltage indicating proteins with optogenetic control of membrane potential using e.g. channelrhodopsin, halorhodopsin, and Archaerhodopsin. If optical measurement and control are combined, one can perform all-optical electrophysiology to probe the dynamic electrical response of any membrane.

The invention provides high-throughput methods of characterizing cells. Robotics and custom software may be used for screening large libraries or large numbers of conditions which are typically encountered in high throughput drug screening methods.

4c. Optogenetic Actuator

In a preferred embodiment, cells are transformed with an optical voltage actuator or light-gated ion channel. The cells comprising the light-gated ion channels act as actuator cells to propagate a signal to the reporter cells expressing the optical voltage reporter such as one of the Arch-based proteins. The far-red excitation spectrum of certain Arch-based reporters suggests that they may be used in an assay with a blue light-activated channelrhodop sin to achieve all-optical electrophysiology. For spatially precise optical excitation, the channelrhodopsin should carry current densities sufficient to induce action potentials (APs) when only a subsection of a cell is excited. Preferably, light used for imaging the reporter should not activate the actuator, and light used for activating the actuator should not confound the fluorescence signal of the reporter. Thus in a preferred embodiment, an optical actuator and an optical reporter are spectrally orthogonal to avoid crosstalk and allow for simultaneous use. Spectrally orthogonal systems are discussed in Carlson and Campbell, 2013, Circular permutated red fluorescent proteins and calcium ion indicators based on mCherry, Protein Eng Des Sel 26(12):763-772.

Any suitable light-gated ion channel or channelrhodopsin may be used as an actuator. Channelrhodopsins are proteins that can give rise to depolarization when activated by light. Channelrhodopsin-2 (ChR2), isolated from the algae Chlamydomonas reinhardtii, can depolarize and evoke precisely timed action potentials. Rhodopsin may refer to a protein that includes an opsin protein and a cofactor, usually retinal (retinaldehyde). The rhodopsin ChR2 is derived from the opsin Channelopsin-2 (Chop2) (Nagel, et. al. Proc. Natl. Acad. Sci. USA 100:13940, incorporated by reference). A light-gated ion channel of the present invention can incorporate retinal that is added or use background levels of retinal present in the cell. It is intended herein that the methods of the invention encompass either the opsin or the rhodopsin form of the protein, e.g. Chop2 or ChR2. Any suitable light-gated ion channel/channelrhodopsion may be used. Other suitable channelrhodopsins include ChR2 point mutants (see e.g., Nagel G, et al. Light activation of channelrhodopsin-2 in excitable cells of Caenorhabditis elegans triggers rapid behavioral responses. Curr. Biol. 2005; 15:2279-2284), channelrhodopsins from other algal species identified using genomic strategies (see e.g., Govorunova et al., 2011, New channelrhodopsin with a red-shifted spectrum and rapid kinetics from Mesostigma viride 2:e00115-11) and chimeras constructed by combining channelrhodopsins. See e.g., Wen et al., 2010, Opto-current-clamp actuation of cortical neurons using a strategically designed channelrhodopsin, PLoS ONE 5:e12893, incorporated by reference. Strong channelrhodopsins fall into at least three genetic classes. The first class consists of wild-type ChR2 and ChR2 mutants with several single-amino-acid substitutions: ChR2(H134R) (ChR2R), ChR2(E123A) (ChETAA), ChR2(T159C) (TC 14), ChR2(E123T/T159C) (ChETATC) and ChR2(L132C) (CatCh). Another class includes hybrids formed from combining different segments of ChR1 and ChR2: ChIEF19, which has an I170V amino acid substitution relative to ChR1, channelrhodopsin fast receiver (FR) and channelrhodopsin green receiver (GR). A third class consists of hybrids formed by combining ChR1 and VChR1 (a ChR variant from Volvox carteri), termed C1V1, including the mutants C1V1(E162T) (C1V1T) and C1V1(E122T/E162T) (C1V1TT). Light-gated ion channels are discussed in U.S. Pat. No. 8,906,360 and US Pub. 2014/0324134, both incorporated by reference. For additional background see Mattis et al., 2014, Principles for applying optogenetic tools derived from direct comparative analysis of microbial opsins, Nat Methods 9(2):159-172, incorporated by reference.

Preferably, a genetically-encoded optogenetic actuator is used. One actuator is channelrhodopsin2 H134R, an optogenetic actuator described in Nagel, G. et al., 2005, Light activation of channelrhodopsin-2 in excitable cells of Caenorhabditis elegans triggers rapid behavioral responses, Curr Biol 15:2279-2284.

A screen of plant genomes has identified an optogenetic actuator, Scherffelia dubia ChR (sdChR), derived from a fresh-water green alga first isolated from a small pond in Essex, England. See Klapoetke et al., 2014, Independent optical excitation of distinct neural populations, Nat Meth Advance Online Publication 1-14; see also Melkonian & Preisig, 1986, A light and electron microscopic study of Scherffelia dubia, a new member of the scaly green flagellates (Prasinophyceae). Nord. J. Bot. 6:235-256, both incorporated by reference. SdChR may offer good sensitivity and a blue action spectrum.

An improved version of sdChR dubbed CheRiff may be used as an optical actuator. The gene for Scherffelia dubia Channelrhodopsin (sdChR) (selected from a screen of channelrhodopsins for its blue excitation peak (474 nm) and its large photocurrent relative to ChR2) is synthesized with mouse codon optimization, a trafficking sequence from Kir2.1 is added to improve trafficking, and the mutation E154A is introduced. CheRiff exhibits significantly decreased crosstalk from red illumination (to 10.5±2.8 pA) allowing its use in cells along with optogenetic reporters described herein. CheRiff shows good expression and membrane trafficking in cultured rat hippocampal neurons. The maximum photocurrent under saturating illumination (488 nm, 500 mW/cm2) is 2.0±0.1 nA (n=10 cells), approximately 2-fold larger than the peak photocurrents of ChR2 H134R or ChIEF (Lin et al., 2009, Characterization of engineered channelrhodopsin variants with improved properties and kinetics, Biophys J 96:1803-1814). In neurons expressing CheRiff, whole-cell illumination at only 22±10 mW/cm2 induces a photocurrent of 1 nA. Compared to ChR2 H134R and to ChIEF under standard channelrhodopsin illumination conditions (488 nm, 500 mW/cm2). At 23° C., CheRiff reaches peak photocurrent in 4.5±0.3 ms (n=10 cells). After a 5 ms illumination pulse, the channel closing time constant was comparable between CheRiff and ChIEF (16±0.8 ms, n=9 cells, and 15±2 ms, n=6 cells, respectively, p=0.94), and faster than ChR2 H134R (25±4 ms, n=6 cells, p<0.05). Under continuous illumination CheRiff partially desensitizes with a time constant of 400 ms, reaching a steady-state current of 1.3±0.08 nA (n=10 cells). Illumination of neurons expressing CheRiff induces trains of APs with high reliability and high repetition-rate.

When testing for optical crosstalk between Arch-based reporters and CheRiff in cultured neurons, illumination sufficient to induce high-frequency trains of APs (488 nm, 140 mW/cm²) perturbed fluorescence of reporters by <1%. Illumination with high intensity red light (640 nm, 900 W/cm²) induced an inward photocurrent through CheRiff of 14.3±3.1 pA, which depolarized neurons by 3.1±0.2 mV (n=5 cells). ChIEF and ChR2 H134R generated similar red light photocurrents and depolarizations. For most applications this level of optical crosstalk is acceptable.

4d. Vectors for Delivery of Optogenetic Systems

The optogenetic reporters and actuators may be delivered in constructs described here as Optopatch constructs delivered through the use of an expression vector. Optopatch may be taken to refer to systems that perform functions traditionally associated with patch clamps, but via an optical input, readout, or both as provided for by, for example, an optical reporter or actuator. An Optopatch construct may include a bicistronic vector for co-expression of CheRiff-eGFP and a reporter (e.g., a suitable Arch-based reporter such as QuasAr2). The reporter and CheRiff constructs may be delivered separately, or a bicistronic expression vector may be used to obtain a uniform ratio of actuator to reporter expression levels.

The genetically encoded reporter, actuator, or both may be delivered by any suitable expression vector using methods known in the art. An expression vector is a specialized vector that contains the necessary regulatory regions needed for expression of a gene of interest in a host cell. Examples of vectors include plasmids (e.g. pBADTOPO, pCI-Neo, pcDNA3.0), cosmids, and viruses (such as a lentivirus, an adeno-associated virus, or a baculovirus). In some embodiments the gene of interest is operably linked to another sequence in the vector. In some embodiments, it is preferred that the viral vectors are replication defective, which can be achieved for example by removing all viral nucleic acids that encode for replication. A replication defective viral vector will still retain its infective properties and enters the cells in a similar manner as a replicating vector, however once admitted to the cell a replication defective viral vector does not reproduce or multiply. The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

Many viral vectors or virus-associated vectors are known in the art. Such vectors can be used as carriers of a nucleic acid construct into the cell. Constructs may be integrated and packaged into non-replicating, defective viral genomes like Adenovirus, Adeno-associated virus (AAV), serotypes of AAV that include AAV1-AAV9, or Herpes simplex virus (HSV) or others, including retroviral and lentiviral vectors, for infection or transduction into cells. The vector may or may not be incorporated into the cell's genome. The constructs may include viral sequences for transfection, if desired. Suitable delivery methods include viral and non-viral vectors, as well as biological or chemical methods of transfection. The methods can yield either stable or transient gene expression in the system used. In some embodiments, a viral vector such as an (i) adenovirus, (ii) adeno-associated virus, (iii) retrovirus, (iv) lentivirus, or (v) other is used.

### (i) Adenovirus

Adenoviruses are double stranded, non-enveloped and icosahedral viruses containing a 36 kb viral genome (Kojaoghlanian et al., 2003, The impact of adenovirus infection on the immunocompromised host, Rev Med Virol 13:155-171). Their genes are divided into early (E1A, E1B, E2, E3, E4), delayed (IX, IVa2) and major late (L1, L2, L3, L4, L5) genes depending on whether their expression occurs before or after DNA replication. More than 51 human adenovirus serotypes have been described which can infect and replicate in a wide range of organs. These viruses have been used to generate a series of vectors for gene transfer cellular engineering. The initial generation of adenovirus vectors were produced by deleting the E1 gene (required for viral replication) generating a vector with a 4 kb cloning capacity. An additional deletion of E3 (responsible for host immune response) allowed an 8 kb cloning capacity (Bett et al., 1994, An efficient and flexible system for construction of adenovirus vectors with insertions or deletions in early regions 1 and 3, PNAS 91:8802-8806; Danthinne and Imperiale, 2000, Production of first generation adenovirus vector, a review, Gene Ther 7:1707-1714). The second generation of vectors was produced by deleting the E2 region (required for viral replication) and/or the E4 region (participating in inhibition of host cell apoptosis) in conjunction with E1 or E3 deletions. The resultant vectors have a cloning capacity of 10-13 kb (Armentano et al., 1995, Characterization of an adenovirus gene transfer vector containing an E4 deletion, Hum Gen Ther 6(10):1343-1353). The third “gutted” generation of vectors was produced by deletion of the entire viral sequence with the exception of the inverted terminal repeats (ITRs) and the cis acting packaging signals. These vectors have a cloning capacity of 25 kb (Kochanek et al., 2001, High-capacity “gutless” adenoviral vectors, Curr Op Mol Ther 3:454-463) and have retained their high transfection efficiency both in quiescent and dividing cells.

Importantly, the adenovirus vectors do not normally integrate into the genome of the host cell, but they have shown efficacy for transient gene delivery into adult stem cells. These vectors have a series of advantages and disadvantages. An important advantage is that they can be amplified at high titers and can infect a wide range of cells. The vectors are generally easy to handle due to their stability in various storing conditions. Adenovirus type 5 (Ad5) has been successfully used in delivering genes in human and mouse stem cells and without integration generally provides transient expression.

### (ii) Adeno-Associated Virus

Adeno-Associated viruses (AAV) are ubiquitous, noncytopathic, replication-incompetent members of ssDNA animal virus of parvoviridae family (Gao et al., 2005, New recombinant serotypes of AAV vectors, Curr Gene Ther 5 (3):285-97). AAV is a small icosahedral virus with a 4.7 kb genome. These viruses have a characteristic termini consisting of palindromic repeats that fold into a hairpin. They replicate with the help of helper virus, which are usually one of the many serotypes of adenovirus. In the absence of helper virus they integrate into the human genome at a specific locus (AAVS1) on chromosome 19 and persist in latent form until helper virus infection occurs. AAV can transduce cell types from different species including mouse, rat and monkey. These viruses are similar to adenoviruses in that they are able to infect a wide range of dividing and non-dividing cells. Unlike adenovirus, they have the ability to integrate into the host genome at a specific site in the human genome.

In some embodiments the viral vector is an adeno-associated virus (AAV) vector. AAV can infect both dividing and non-dividing cells and may incorporate its genome into that of the host cell. One suitable viral vector uses recombinant adeno-associated virus (rAAV), which is widely used for gene delivery in the CNS. In certain embodiments, the vector may use AAV serotype 9 (AAV9). See Bell et al., 2011, The AAV9 receptor and its modification to improve in vivo lung gene transfer in mice, J Clin Invest 121(6):2427-2435; and Cearley & Wolfe, 2006, Transduction characteristics of adeno-associated virus vectors expressing cap serotypes 7, 8, 9, and Rh10 in the mouse brain, Mol Ther 13:528-537; and Foust et al., 2009, Intravascular AAV9 preferentially targets neonatal neurons and adult astrocytes, Nat Biotechnol 27:59-65.

### (ii) Retroviruses

Retroviral genomes consist of two identical copies of single stranded positive sense RNAs, 7-10 kb in length coding for three genes; gag, pol and env, flanked by long terminal repeats (LTR) (Yu & Schaffer, 2006, Engineering retroviral and lentiviral vectors by selection of a novel peptide insertion library for enhanced purification, J. Virol. 80:3285-3292). The gag gene encodes the core protein capsid containing matrix and nucleocapsid elements that are cleavage products of the gag precursor protein. The pol gene codes for the viral protease, reverse transcriptase and integrase enzymes derived from gag-pol precursor gene. The env gene encodes the envelop glycoprotein which mediates viral entry. An important feature of the retroviral genome is the presence of LTRs at each end of the genome. These sequences facilitate the initiation of viral DNA synthesis, moderate integration of the proviral DNA into the host genome, and act as promoters in regulation of viral gene transcription. Retroviruses are subdivided into three general groups: the oncoretroviruses (Maloney Murine Leukenmia Virus, MoMLV), the lentiviruses (HIV), and the spumaviruses (foamy virus). Retroviral based vectors are the most commonly used integrating vectors for gene therapy. These vectors generally have a cloning capacity of approximately 8 kb and are generated by a complete deletion of the viral sequence with the exception of the LTRs and the cis acting packaging signals.

### (ii) Lentivirus

Lentiviruses are members of Retroviridae family of viruses (Scherr et al., 2002, Gene transfer into hematopoietic stem cells using lentiviral vectors, Curr Gene Ther. 2(1):45-55). They have a more complex genome and replication cycle as compared to the oncoretroviruses (Beyer et al., 2002, Oncoretrovirus and lentivirus vectors pseudotyped with lymphocytic choriomeningitis virus glycoprotein: generation, concentration, and broad host range, J. Virol 76:1488-1495). They differ from simpler retroviruses in that they possess additional regulatory genes and elements, such as the tat gene, which mediates the transactivation of viral transcription and rev, which mediates nuclear export of un-spliced viral RNA. See also U.S. Pat. No. 5,665,577 to Sodroski, the contents of which are incorporated by reference.

Lentivirus vectors are derived from the human immunodeficiency virus (HIV-1) by removing the genes necessary for viral replication rendering the virus inert. Although they are devoid of replication genes, the vector can still efficiently integrate into the host genome allowing stable expression of the transgene. These vectors have the additional advantage of a low cytotoxicity and an ability to infect diverse cell types.

Lentiviral vectors may include a eukaryotic promoter. The promoter can be any inducible promoter, including synthetic promoters, that can function as a promoter in a eukaryotic cell. For example, the eukaryotic promoter can be, but is not limited to, CamKIIα promoter, human Synapsin promoter, ecdysone inducible promoters, E1a inducible promoters, tetracycline inducible promoters etc., as are well known in the art. In addition, the lentiviral vectors used herein can further comprise a selectable marker, which can comprise a promoter and a coding sequence for a selectable trait. Nucleotide sequences encoding selectable markers are well known in the art, and include those that encode gene products conferring resistance to antibiotics or anti-metabolites, or that supply an auxotrophic requirement. Examples of such sequences include, but are not limited to, those that encode thymidine kinase activity, or resistance to methotrexate, ampicillin, kanamycin, among others. Use of lentiviral vectors is discussed in Wardill et al., 2013, A neuron-based screening platform for optimizing genetically-encoded calcium indicators, PLoS One 8(10):e77728; Dottori, et al., Neural development in human embryonic stem cells-applications of lentiviral vectors, J Cell Biochem 112(8):1955-62; and Diester et al., 2011, An optogenetic toolbox designed for primates, Nat Neurosci 14(3):387-97. When expressed under a CaMKIIα promoter in cultured rat hippocampal neurons the Optopatch construct exhibits high expression and good membrane trafficking of both CheRiff and a reporter.

In certain embodiments, genetic material is delivered by a non-viral method. Non-viral methods include plasmid transfer, modified RNA, and the application of targeted gene integration through the use of integrase or transposase technologies. Exemplary recombinase systems include: cre recombinase from phage P1 (Lakso et al., 1992, Targeted oncogene activation by site-specific recombination in transgenic mice, PNAS 89:6232-6236; Orban et al., 1992, Tissue- and site-specific DNA recombination in transgenic mice, PNAS 89:6861-6865), FLP (flippase) from yeast 2 micron plasmid (Dymecki, 1998, Using Flp-recombinase to characterize expansion of Wnt1-expressing neural progenitors in the mouse, Dev Biol 201:57-65), and an integrase isolated from streptomyses phage I C31 (Groth et al., 2000, A phage integrase directs efficient site-specific integration in human cells, PNAS 97(11):5995-6000). Each of these recombinases recognize specific target integration sites. Cre and FLP recombinase catalyze integration at a 34 bp palindromic sequence called lox P (locus for crossover) and FRT (FLP recombinase target) respectively. Phage integrase catalyzes site-specific, unidirectional recombination between two short att recognition sites in mammalian genomes. Recombination results in integration when the att sites are present on two different DNA molecules and deletion or inversion when the att sites are on the same molecule. It has been found to function in tissue culture cells (in vitro) as well as in mice (in vivo).

The Sleeping Beauty (SB) transposon is comprised of two inverted terminal repeats of 340 base pairs each (Izsvak et al., 2000, Sleeping Beauty, a wide host-range transposon vector for genetic transformation in vertebrates, J Mol Biol 302(1):93-102). This system directs the precise transfer of specific constructs from a donor plasmid into a mammalian chromosome. The excision and integration of the transposon from a plasmid vector into a chromosomal site is mediated by the SB transposase, which can be delivered to cells as either in a cis or trans manner (Kaminski et al., 2002, Design of a nonviral vector for site-selective, efficient integration into the human genome, FASEB J 6:1242-1247). A gene in a chromosomally integrated transposon can be expressed over the lifetime of a cell. SB transposons integrate randomly at TA-dinucleotide base pairs although the flanking sequences can influence integration.

In certain embodiments, methods of the invention use a Cre-dependent expression system. Cre-dependent expression includes Cre-Lox recombination, a site-specific recombinase technology that uses the enzyme Cre recombinase, which recombines a pair of short target sequences called the Lox sequences. This system can be implemented without inserting any extra supporting proteins or sequences. The Cre enzyme and the original Lox site called the LoxP sequence are derived from bacteriophage P1. Bacteriophage P1 uses Cre-lox recombination to circularize and replicate its genomic DNA. This recombination strategy is employed in Cre-Lox technology for genome manipulation, which requires only the Cre recombinase and LoxP sites. Sauer & Henderson, 1988, Site-specific DNA recombination in mammalian cells by the Cre recombinase of bacteriophage P1, PNAS 85:5166-70 and Sternberg & Hamilton, 1981, Bacteriophage P1 site-specific recombination. I. Recombination between LoxP sites, J Mol Biol 150:467-86. Methods may use a Cre recombinase-dependent viral vector for targeting tools such as channelrhodopsin-2 (ChR2) to specific neurons with expression levels sufficient to permit reliable photostimulation. Optogenetic tools such as ChR2 tagged with a fluorescent protein such as mCherry (e.g., ChR2mCherry) or any other of the tools discussed herein are thus delivered to the cell or cells for use in characterizing those cells.

The delivery vector may include Cre and Lox. The vector may further optionally include a Lox-stop-Lox (LSL) cassette to prevent expression of the transgene in the absence of Cre-mediated recombination. In the presence of Cre recombinase, the LoxP sites recombine, and a removable transcription termination Stop element is deleted. Removal of the stop element may be achieved through the use of AdenoCre, which allows control of the timing and location of expression. Use of the LSL cassette is discussed in Jackson, et al., 2001, Analysis of lung tumor initiation and progression using conditional expression of oncogenic K-ras, Genes & Dev 15:3243-3248.

In certain embodiments, a construct of the invention uses a “flip-excision” switch, or FLEX switch (FLip EXicision), to achieve stable transgene inversion. The FLEX switch is discussed in Schnutgen et al., 2003, A directional strategy for monitoring Cre-mediated recombination at the cellular level in the mouse, Nat Biotechnol 21:562-565. The FLEX switch uses two pairs of heterotypic, antiparallel LoxP-type recombination sites which first undergo an inversion of the coding sequence followed by excision of two sites, leading to one of each orthogonal recombination site oppositely oriented and incapable of further recombination. A FLEX switch provides high efficiency and irreversibility. Thus in some embodiments, methods use a viral vector comprising rAAV-FLEX-rev-ChR2mCherry. Additionally or alternatively, a vector may include FLEX and any other optogenetic tool discussed herein (e.g., rAAV-FLEX-QuasAr2, rAAV-FLEX-CheRiff). Using rAAV-FLEX-rev-ChR2mCherry as an illustrative example, Cre-mediated inversion of the ChR2mCherry coding sequence results in the coding sequence being in the wrong orientation (i.e., rev-ChR2mCherry) for transcription until Cre inverts the sequence, turning on transcription of ChR2mCherry. FLEX switch vectors are discussed in Atasoy et al., 2009, A FLEX switch targets channelrhodopsin-2 to multiple cell types for imaging and long-range circuit mapping, J Neurosci 28(28):7025-7030.

Use of a viral vector such as Cre-Lox system with an optical reporter, optical actuator, or both (optionally with a FLEX switch and/or a Lox-Stop-Lox cassette) for labeling and stimulation of neurons allows for efficient photo-stimulation with only brief exposure (1 ms) to less than 100 μW focused laser light or to light from an optical fiber. Such Further discussion may be found in Yizhar et al., 2011, Optogenetics in neural systems, Neuron 71(1):9-34; Cardin et al., 2010, Targeted optogenetic stimulation and recording of neurons in vivo using cell-type-specific expression of Channelrhodopsin-2, Nat Protoc 5(2):247-54; Rothermel et al., 2013, Transgene expression in target-defined neuron populations mediated by retrograde infection ith adeno-associated viral vectors, J Neurosci 33(38):195-206; and Saunders et al., 2012, Novel recombinant adeno-associated viruses for Cre activated and inactivated transgene expression in neurons, Front Neural Circuits 6:47.

In certain embodiments, actuators, reporters, or other genetic material may be delivered using chemically-modified mRNA. It may be found and exploited that certain nucleotide modifications interfere with interactions between mRNA and toll-like receptor, retinoid-inducible gene, or both. Exposure to mRNAs coding for the desired product may lead to a desired level of expression of the product in the cells. See, e.g., Kormann et al., 2011, Expression of therapeutic proteins after delivery of chemically modified mRNA in mice, Nat Biotech 29(2):154-7; Zangi et al., 2013, Modified mRNA directs the fate of heart progenitor cells and induces vascular regeneration after myocardial infarction, Nat Biotech 31:898-907.

It may be beneficial to culture or mature the cells after transformation with the genetically encoded optical reporter with optional actuator. In some embodiments, the neurons are matured for 8-10 days post infection. Using microscopy and analytical methods described herein, the cell and its action potentials may be observed. For additional discussion, see U.S. Pub. 2013/0224756, incorporated by reference in its entirety for all purposes.

Other methods for transfection include physical methods such as electroporation as well as methods that employ biomolecules.

Electroporation relies on the use of brief, high voltage electric pulses which create transient pores in the membrane by overcoming its capacitance. One advantage of this method is that it can be utilized for both stable and transient gene expression in most cell types. The technology relies on the relatively weak nature of the hydrophobic and hydrophilic interactions in the phospholipid membrane and its ability to recover its original state after the disturbance. Once the membrane is permeabilized, polar molecules can be delivered into the cell with high efficiency. Large charged molecules like DNA and RNA move into the cell through a process driven by their electrophoretic gradient.

Biomolecule-based methods include the use of protein transduction domains (PTD). PTDs are short peptides that are transported into the cell without the use of the endocytotic pathway or protein channels. The mechanism involved in their entry is not well understood, but it can occur even at low temperature (Derossi et al., 1996, J Biol Chem 271(30):18188-93). The two most commonly used naturally occurring PTDs are the trans-activating activator of transcription domain (TAT) of human immunodeficiency virus and the homeodomain of Antennapedia transcription factor. In addition to these naturally occurring PTDs, there are a number of artificial peptides that have the ability to spontaneously cross the cell membrane (Joliot and Prochiantz, 2004, Transduction peptides: from technology to physiology, Nat Cell Biol 6(3):189-96). These peptides can be covalently linked to the pseudo-peptide backbone of PNA (peptide nucleic acids) to help deliver them into the cell.

Additionally or alternatively, liposomes may be used. Liposomes are synthetic vesicles that resemble the cell membrane. When lipid molecules are agitated with water they spontaneously form spherical double membrane compartments surrounding an aqueous center forming liposomes. They can fuse with cells and allow the transfer of “packaged” material into the cell. Liposomes have been successfully used to deliver genes, drugs, reporter proteins and other biomolecules into cells (Felnerova et al., 2004, Liposomes and virosomes as delivery systems for antigens, nucleic acids and drugs, Curr Opin Biotech 15: 518-529). The advantage of liposomes is that they are made of natural biomolecules (lipids) and are nonimmunogenic.

Diverse hydrophilic molecules can be incorporated into them during formation. For example, when lipids with positively charged head group are mixed with recombinant DNA they can form lipoplexes in which the negatively charged DNA is complexed with the positive head groups of lipid molecules. These complexes can then enter the cell through the endocytotic pathway and deliver the DNA into lysosomal compartments. The DNA molecules can escape this compartment with the help of dioleoylethanolamine (DOPE) and are transported into the nucleus where they can be transcribed (Tranchant et al., 2004, Physicochemical optimisation of plasmid delivery by cationic lipids, J Gene Med 6 Suppl 1:S24-35).

Immunoliposomes are liposomes with specific antibodies inserted into their membranes. The antibodies bind selectively to specific surface molecules on the target cell to facilitate uptake. The surface molecules targeted by the antibodies are those that are preferably internalized by the cells so that upon binding, the whole complex is taken up. This approach increases the efficiency of transfection by enhancing the intracellular release of liposomal components. These antibodies can be inserted in the liposomal surface through various lipid anchors or attached at the terminus of polyethylene glycol grafted onto the liposomal surface. In addition to providing specificity to gene delivery, the antibodies can also provide a protective covering to the liposomes that helps to limit their degradation after uptake by endogenous RNAses or proteinases (Bendas, 2001, Immunoliposomes: A promising approach to targeting cancer therapy, BioDrugs 15(4):215-224). To further prevent degradation of liposomes and their contents in the lysosomal compartment, pH sensitive immunoliposomes can be employed (Torchilin et al., 2006, pH-sensitive liposomes, J Liposome Res 3:201-255). These liposomes enhance the release of liposomal content into the cytosol by fusing with the endosomal membrane within the organelle as they become destabilized and prone to fusion at acidic pH.

In general, non-viral gene delivery systems have not been as widely applied as a means of gene delivery into stem cells as viral gene delivery systems. However, promising results are demonstrated in a study looking at the transfection viability, proliferation and differentiation of adult neural stem/progenitor cells into the three neural lineages neurons. Non-viral, non-liposomal gene delivery systems (ExGen500 and FuGene6) had a transfection efficiency of between 16% (ExGen500) and 11% (FuGene6) of cells. FuGene6-treated cells did not differ from untransfected cells in their viability or rate of proliferation, whereas these characteristics were significantly reduced following ExGen500 transfection. Importantly, neither agent affected the pattern of differentiation following transfection. Both agents could be used to genetically label cells, and track their differentiation into the three neural lineages, after grafting onto ex vivo organotypic hippocampal slice cultures (Tinsley et al, 2006, Efficient non-viral transfection of adult neural stem/progenitor cells, without affecting viability, proliferation or differentiation, J Gene Med 8(1):72-81).

### (iv) Polymer-Based Methods

The protonated epsilon-amino groups of poly L-lysine (PLL) interact with the negatively charged DNA molecules to form complexes that can be used for gene delivery. These complexes can be rather unstable and showed a tendency to aggregate. The conjugation of polyethylene glycol (PEG) was found to lead to an increased stability of the complexes. To confer a degree of tissue-specificity, targeting molecules such as tissue-specific antibodies have also been employed. An additional gene carrier that has been used for transfecting cells is polyethylenimine (PEI) which also forms complexes with DNA. Due to the presence of amines with different pKa values, it has the ability to escape the endosomal compartment. PEG grafted onto PEI complexes was found to reduce the cytotoxicity and aggregation of these complexes. This can also be used in combination with conjugated antibodies to confer tissue-specificity. See Lee & Kim, 2014, Bioreducible polymers for therapeutic gene delivery, J Control Relase ePub; Wang et al., 2013, Non-viral gene delivery methods, Curr Pharm Biotechnol 14(1):46-40; and Gupta et al., 2012, Structuring polymers for delivery of DNA-based therapeutics: updated insights, Crit Rev Ther Drug Carrier Syst 29(6):447-85.

Optical actuators, reporters, or both as discussed herein may be targeted to intracellular organelles, including mitochondria, the endoplasmic reticulum, the sarcoplasmic reticulum, synaptic vesicles, and phagosomes. Accordingly, in one embodiment, the invention provides expression constructs, such as viral constructs comprising a reporter and/or actuatory operably linked to a sequence targeting the protein to an intracellular organelle, including a mitochondrion, an endoplasmic reticulum, a sarcoplasmic reticulum, a synaptic vesicle, and a phagosome. In some embodiments, the optical voltage sensor further comprises a localization or targeting sequence to direct or sort the sensor to a particular face of a biological membrane or subcellular organelle.

Methods of the invention can be used to express proteins transiently, stably, or both. Transduction and transformation methods for transient expression of nucleic acids are well known to one skilled in the art. Transient transfection can be carried out, e.g., using calcium phosphate, by electroporation, or by mixing a cationic lipid with the material to produce liposomes, cationic polymers or highly branched organic compounds. All these are in routine use in genetic engineering.

Exemplary protocols for stable expression can be found, e.g., in Essential Stem Cell Methods, edited by Lanza and Klimanskaya, published in 2008, Academic Press. For example, one can generate a virus that integrates into the genome and comprises a selectable marker, and infect the cells with the virus and screen for cells that express the marker, which cells are the ones that have incorporated the virus into their genome. A VSV-g psuedotyped lenti virus with a puromycin selectable marker in HEK cells can be used according to established procedures. Generally, one can use a stem cell specific promoter to encode a GFP if FACS sorting is necessary. The hiPS cultures are cultivated on embryonic fibroblast (EF) feeder layers or on Matrigel in fibroblast growth factor supplemented EF conditioned medium. The cells are dissociated by trypsinization, plated, and maintained in an undifferentiated state, e.g., using EF conditioned medium. Cells are cultured with the virus for 24 hours; washed, typically with PBS, and fresh media is added with a selection marker, such as 1 micro g/mL puromycin. The medium is replaced about every 2 days with additional puromycin. Cells surviving after 1 week are re-plated, e.g., using the hanging drop method to form EBs with stable incorporation of gene.

In some embodiments, it is advantageous to express an optical voltage reporter (e.g., QuasAr2 or a suitable variant thereof) in only a single cell-type within an organism, and further, if desired, to direct the reporter to a particular subcellular structure within the cell. Upstream promoters control when and where the gene is expressed. Constructs are made that optimize expression in all eukaryotic cells. In one embodiment, the optical voltage sensor is under the control of a neuron-specific promoter.

The promoter sequence can be selected to restrict expression of the protein to a specific class of cells and environmental conditions. Common promoter sequences include, but are not limited to, CMV (cytomegalovirus promoter; a universal promoter for mammalian cells), 14x UAS-E1b (in combination with the transactivator Gal4, this promoter allows combinatorial control of transgene expression in a wide array of eukaryotes. Tissue-specific expression can be achieved by placing Gal4 under an appropriate promoter, and then using Gal4 to drive the UAS-controlled transgene), HuC (drives pan-neuronal expression in zebrafish and other teleosts), ara (allows regulation of expression with arabinose in bacteria) and lac (allows regulation of expression with IPTG in bacteria).

Methods of the invention can be used to target actuators, reporters, or both to specific cellular sites such as the plasma membrane. In some embodiments, constructs are designed to include signaling sequences to optimize localization of the protein to the plasma membrane. These can include e.g., a C-terminal signaling sequence from the O.sub.2 nicotinic acetylcholine receptor and/or an endoplasmic reticulum export motif from Kir2.1.

Additional improvements in plasma localization can be obtained by adding Golgi export sequences and membrane localization sequences. See Gong et al., 2014, Imaging neural spiking in brain tissue using FRET-opsin protein voltage sensors, Nat Comm 5:articel3674; and Gradinaru et al., 2010, Molecular and Cellular Approaches for Diversifying and Extending Optogenetics, Cell 141:154-165.

As discussed above, the invention includes optogenetic reporters, optogenetic actuators, and vectors for the expression of microbial rhodopsins. See also U.S. Pat. No. 8,716,447 to Deisseroth; U.S. Pat. No. 8,647,870 to Hegemann; U.S. Pat. No. 8,617,876 to Farrar; U.S. Pat. No. 8,603,790 to Deisseroth; U.S. Pat. No. 8,580,937 to Spudich; U.S. Pat. No. 8,562,658 to Shoham; and U.S. Pat. No. 8,202,699 to Hegemann, the contents of each of which are incorporated by reference.

The invention further provides cells expressing the constructs, and further methods of measuring membrane potential changes in the cells expressing such constructs as well as methods of screening for agents that affect the membrane potential of one or more of the intracellular membranes.

### 5. Imaging Activity Assay

5a. Capturing Images

Methods of the invention may include stimulating the cells that are to be observed. Stimulation may be direct or indirect (e.g., optical stimulation of an optical actuator or stimulating an upstream cell in synaptic or gap junction-mediated communication with the cell(s) to be observed). Stimulation may be optical, electrical, chemical, or by any other suitable method. Stimulation may involve any pattern of a stimulation including, for example, regular, periodic pulses, single pulses, irregular patterns, or any suitable pattern. Methods may include varying optical stimulation patterns in space or time to highlight particular aspects of cellular function. For example, a pulse pattern may have an increasing frequency. In certain embodiments, imaging includes stimulating a neuron that expresses an optical actuator using pulses of light.

Optical reporters of the invention provide accurate values of the membrane potential, without systematic artifacts from photobleaching, variation in illumination intensity, cell movement, or variations in protein expression level. In cells that are accessible to patch clamp, one can calibrate the fluorescence as a function of membrane potential by varying the membrane potential under external control. However, constructs of the invention function in systems that are inaccessible to patch clamp. In these cases direct calibration is not possible.

The Arch 3 fusion with eGFP enables ratiometric determination of membrane potential. Similar ratiometric determinations may be made using other optical reporters such as those described in this application using the identical concept. The eGFP fluorescence is independent of membrane potential, The ratio of Arch 3 fluorescence to eGFP fluorescence provides a measure of membrane potential that is independent of variations in expression level, illumination, or movement.

In the methods of the invention, the cells are excited with a light source so that the emitted fluorescence can be detected. The wavelength of the excitation light depends on the fluorescent molecule. For example, the Archaerhodopsin constructs in the examples are all excitable using light with wavelengths varying between lambda=594 nm and lambda=645 nm. Alternatively, the range may be between lambda=630-645 nm. For example a commonly used Helium Neon laser emits at lambda=632.8 nm and can be used in excitation of the fluorescent emission of these molecules.

In some embodiments a second light is used. For example, if the cell expresses a reference fluorescent molecule or a fluorescent molecule that is used to detect another feature of the cell, such a pH or Calcium concentration. In such case, the second wavelength differs from the first wavelength. Examples of useful wavelengths include wavelengths in the range of lambda=447-594 nm, for example, lambda=473 nm, lambda=488 nm, lambda=514 nm, lambda=532 nm, and lambda=561 nm.

Methods of the invention allow for the measurement of action potentials with sub-millisecond temporal resolution. A neuron expressing an Optopatch construct may be exposed to whole-field illumination with pulses of blue light (10 ms, 25 mW/cm²) to stimulate CheRiff, and simultaneous constant illumination with red light (800 W/cm²) to excite fluorescence of the reporter (e.g., QuasAr2 or a suitable variant thereof). The fluorescence of the reporter may be imaged at a 1 kHz frame rate. Key parameters include temporal precision with which single spikes can be elicited and recorded, signal-to-noise ratio (SNR) in fluorescence traces, and long-term stability of the reporter signal. Methods provided herein may be found to optimize those parameters. Further discussion may be found in Foust et al., 2010, Action potentials initiate in the axon initial segment and propagate through axon collaterals reliably in cerebellar Purkinje neurons, J. Neurosci 30:6891-6902; and Popovic et al., 2011, The spatio-temporal characteristics of action potential initiation in layer 5 pyramidal neurons: a voltage imaging study, J. Physiol. 589:4167-4187.

In some embodiments, measurements are made using a low-magnification microscope that images a 1.2×3.3 mm field of view with 3.25 μm spatial resolution and 2 ms temporal resolution. In other embodiments, measurements are made using a high-magnification microscope that images a 100 μm field of view with 0.8 μm spatial resolution and 1 ms temporal resolution. A suitable instrument is an inverted fluorescence microscope, similar to the one described in the Supplementary Material to Kralj et al., 2012, Optical recording of action potentials in mammalian neurons using a microbial rhodopsin, Nat. Methods 9:90-95. Briefly, illumination from a red laser 640 nm, 140 mW (Coherent Obis 637-140 LX), is expanded and focused onto the back-focal plane of a 60× oil immersion objective, numerical aperture 1.45 (Olympus 1-U2B616).

FIG. 10 gives a functional diagram of components of an optical imaging apparatus 501 according to certain embodiments. A 488 nm blue laser beam is modulated in intensity by an acousto-optic modulator (not shown), and then reflected off a digital micromirror device (DMD) 505. The DMD imparted a spatial pattern on the blue laser beam (used for CheRiff excitation) on its way into the microscope. The micromirrors were re-imaged onto the sample 509, leading to an arbitrary user-defined spatiotemporal pattern of illumination at the sample. Simultaneous whole-field illumination with 640 nm red light excites fluorescence of the reporter.

With the inverted fluorescence microscope, illumination from a blue laser 488 nm 50 mW (Omicron PhoxX) is sent through an acousto-optic modulator (AOM; Gooch and Housego 48058-2.5-0.55-5W) for rapid control over the blue intensity. The beam is then expanded and modulated by DMD 505 with 608×684 pixels (Texas Instruments LightCrafter). The DMD is controlled via custom software (Matlab) through a TCP/IP protocol. The DMD chip is re-imaged through the objective onto the sample, with the blue and red beams merging via a dichroic minor. Each pixel of the DMD corresponds to 0.65 μm in the sample plane. A 532 nm laser is combined with the red and blue beams for imaging of mOrange2. Software is written to map DMD coordinates to camera coordinates, enabling precise optical targeting of any point in the sample.

To achieve precise optical stimulation of user-defined regions of a neuron, pixels on DMD 505 are mapped to pixels on the camera. The DMD projects an array of dots of known dimensions onto the sample. The camera acquires an image of the fluorescence. Custom software locates the centers of the dots in the image, and creates an affine transformation to map DMD coordinates onto camera pixel coordinates.

A dual-band dichroic filter (Chroma zt532/635rpc) separates reporter (e.g., Arch) from excitation light. A 531/40 nm bandpass filter (Semrock FF01-531/40-25) may be used for eGFP imaging; a 710/100 nm bandpass filter (Chroma, HHQ710/100) for Arch imaging; and a quad-band emission filter (Chroma ZET405/488/532/642m) for mOrange2 imaging and pre-measurement calibrations. A variable-zoom camera lens (Sigma 18-200 mm f/3.5-6.3 II DC) is used to image the sample onto an EMCCD camera (Andor iXon+DU-860), with 128×128 pixels. Images may be first acquired at full resolution (128×128 pixels). Data is then acquired with 2×2 pixel binning to achieve a frame rate of 1,000 frames/s. For runs with infrequent stimulation (once every 5 s), the red illumination is only on from 1 s before stimulation to 50 ms after stimulation to minimize photobleaching. Cumulative red light exposure may be limited to <5 min. per neuron.

Low magnification wide-field imaging is performed with a custom microscope system based around a 2×, NA 0.5 objective (Olympus MVX-2). Illumination is provided by six lasers 640 nm, 500 mW (Dragon Lasers 635M500), combined in three groups of two. Illumination is coupled into the sample using a custom fused silica prism, without passing through the objective. Fluorescence is collected by the objective, passed through an emission filter, and imaged onto a scientific CMOS camera (Hamamatsu Orca Flash 4.0). Blue illumination for channelrhodopsin stimulation is provided by a 473 nm, 1 W laser (Dragon Lasers), modulated in intensity by an AOM and spatially by a DMD (Digital Light Innovations DLi4130-ALP HS). The DMD is re-imaged onto the sample via the 2× objective. During a run, neurons may be imaged using wide-field illumination at 488 nm and eGFP fluorescence. A user may select regions of interest on the image of the neuron, and specify a time course for the illumination in each region. The software maps the user-selected pixels onto DMD coordinates and delivers the illumination instructions to the DMD.

The inverted fluorescence micro-imaging system records optically from numerous (e.g., 50) expressing cells or cell clusters in a single field of view. The system may be used to characterize optically evoked firing patterns and AP waveforms in neurons expressing an Optopatch construct. Each field of view is exposed to whole-field pulses of blue light to evoke activity (e.g., 0.5 s, repeated every 6 s, nine intensities increasing from 0 to 10 mW/cm2). Reporter fluorescence such as from QuasAr2 may be simultaneously monitored with whole-field excitation at 640 nm, 100 W/cm2. Additional useful discussion of microscopes and imaging systems may be found in U.S. Pat. No. 8,532,398 to Filkins; U.S. Pat. No. 7,964,853 to Araya; U.S. Pat. No. 7,560,709 to Kimura; U.S. Pat. No. 7,459,333 to Richards; U.S. Pat. No. 6,972,892 to DeSimone; U.S. Pat. No. 6,898,004 to Shimizu; U.S. Pat. No. 6,885,492 to DeSimone; and U.S. Pat. No. 6,243,197 to Schalz, the contents of each of which are incorporated by reference.

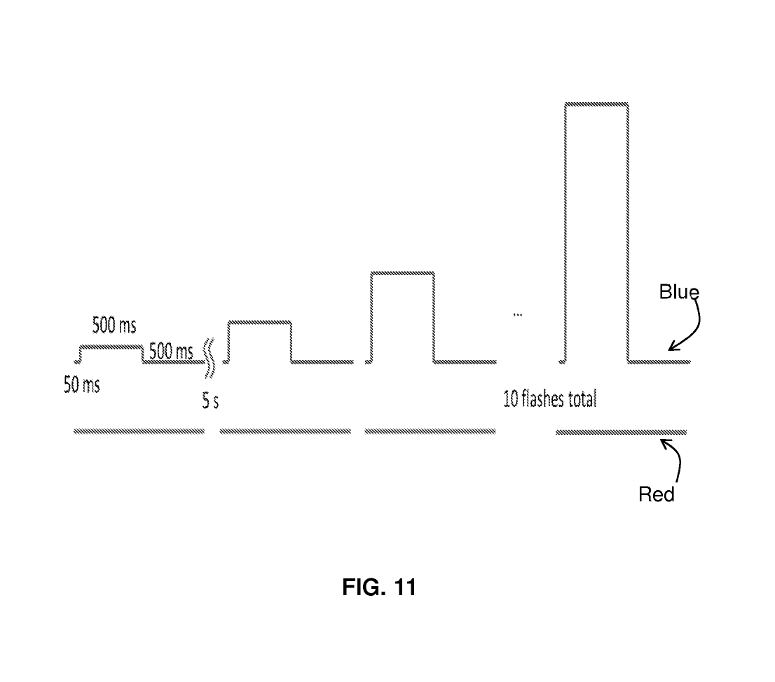

FIG. 11 illustrates a pulse sequence of red and blue light used to record action potentials under increasing optical stimulation. In some embodiments, neurons are imaged on a high resolution microscope with 640 nm laser (600 W/cm²) for voltage imaging. In certain embodiments, neurons are imaged on a high resolution microscope with 640 nm laser (600 W/cm²) for voltage imaging and excited with a 488 nm laser (20-200 mW/cm²). Distinct firing patterns can be observed (e.g., fast adapting and slow-adapting spike trains). System measurements can detect rare electrophysiological phenotypes that might be missed in a manual patch clamp measurement. Specifically, the cells' response to stimulation (e.g., optical actuation) may be observed. Instruments suitable for use or modification for use with methods of the invention are discussed in U.S. Pub. 2013/0170026 to Cohen, incorporated by reference.

Using the described methods, populations of cells may be measured. For example, both diseased and corrected (e.g., by zing finger domains) motor neurons may be measured. A cell's characteristic signature such as a neural response as revealed by a spike train may be observed.

5b. Extracting Fluorescence from Movies

Fluorescence values are extracted from raw movies by any suitable method. One method uses the maximum likelihood pixel weighting algorithm described in Kralj et al., 2012, Optical recording of action potentials in mammalian neurons using a microbial rhodopsin, Nat Methods 9:90-95. Briefly, the fluorescence at each pixel is correlated with the whole-field average fluorescence. Pixels that showed stronger correlation to the mean are preferentially weighted. This algorithm automatically finds the pixels carrying the most information, and de-emphasizes background pixels.

In movies containing multiple cells, fluorescence from each cell is extracted via methods known in the art such as Mukamel et al., 2009, Automated analysis of cellular signals from large-scale calcium imaging data, Neuron 63(6):747-760, or Maruyama et al., 2014, Detecting cells using non-negative matrix factorization on calcium imaging data, Neural Networks 55:11-19. These methods use the spatial and temporal correlation properties of action potential firing events to identify clusters of pixels whose intensities co-vary, and associate such clusters with individual cells.

Alternatively, a user defines a region comprising the cell body and adjacent neurites, and calculates fluorescence from the unweighted mean of pixel values within this region. In low-magnification images, direct averaging and the maximum likelihood pixel weighting approaches may be found to provide optimum signal-to-noise ratios.

### 6. Signal Processing

6a. Independent Component Analysis to Associate Signals with Cells

An image or movie may contain multiple cells in any given field of view, frame, or image. In images containing multiple neurons, the segmentation is performed semi-automatically using an independent components analysis (ICA) based approach modified from that of Mukamel, et al., 2009, Automated analysis of cellular signals from large-scale calcium imaging data, Neuron 63:747-760. The ICA analysis can isolate the image signal of an individual cell from within an image.

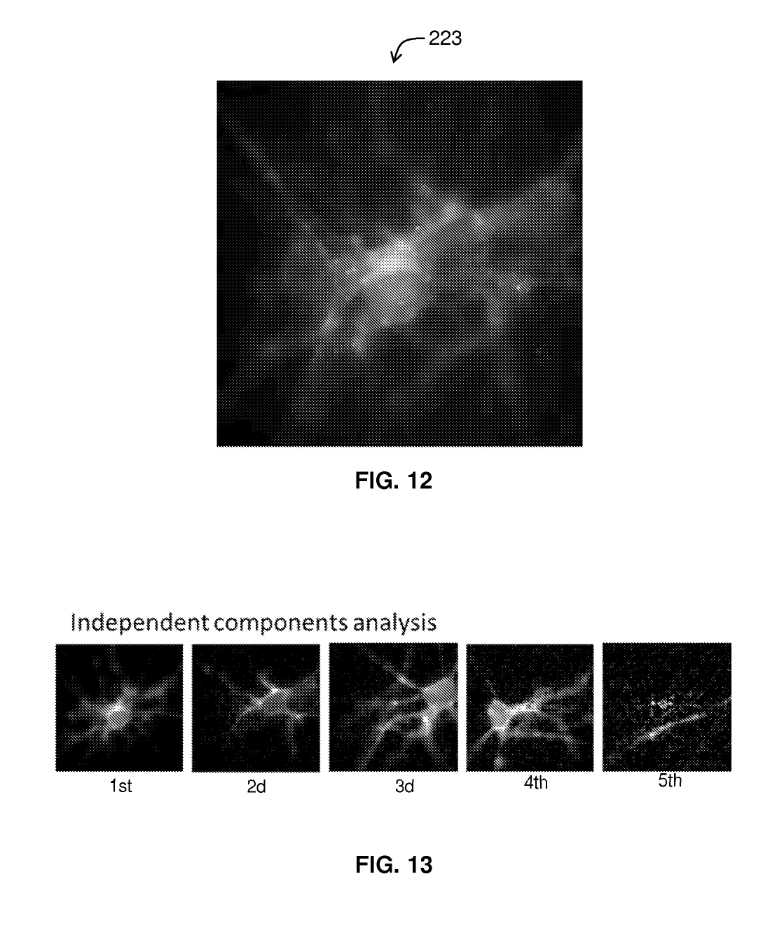

FIG. 12-FIG. 15 illustrate the isolation of individual cells in a field of view. Individual cells are isolated in a field of view using an independent component analysis.

FIG. 12 shows an image that contains five neurons whose images overlap with each other. The fluorescence signal at each pixel is an admixture of the signals from each of the neurons underlying that pixel.

As shown in FIG. 13, the statistical technique of independent components analysis finds clusters of pixels whose intensity is correlated within a cluster, and maximally statistically independent between clusters. These clusters correspond to images of individual cells comprising the aggregate image of FIG. 12.

From the pseudo-inverse of the set of images shown in FIG. 13 are calculated spatial filters with which to extract the fluorescence intensity time-traces for each cell. Filters are created by setting all pixel weights to zero, except for those in one of the image segments. These pixels are assigned the same weight they had in the original ICA spatial filter.

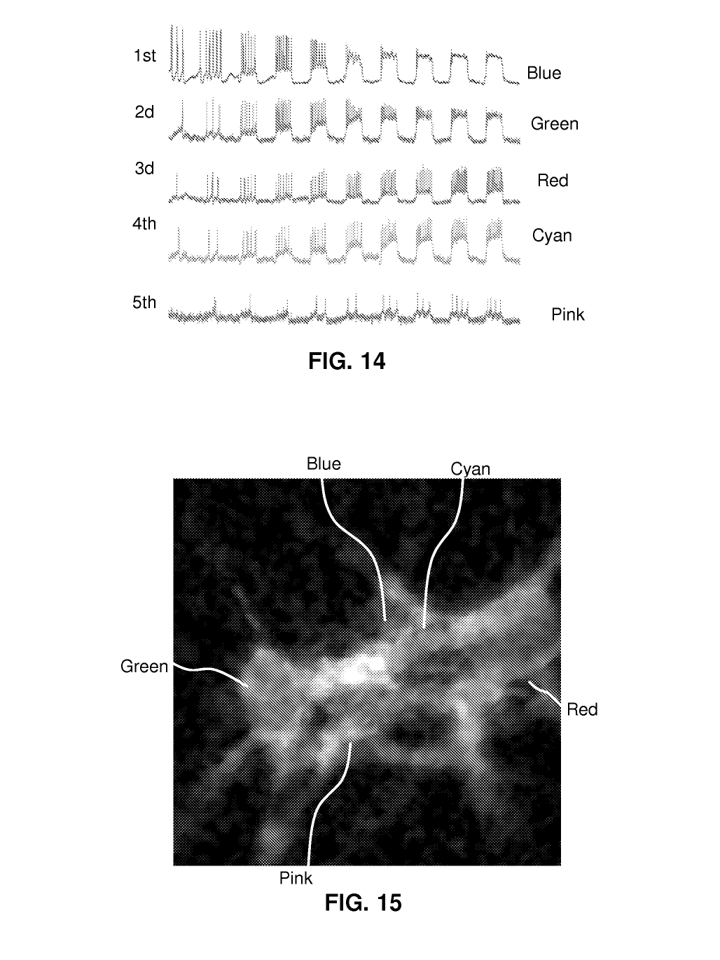

In FIG. 14, by applying the segmented spatial filters to the movie data, the ICA time course has been broken into distinct contributions from each cell. Segmentation may reveal that the activities of the cells are strongly correlated, as expected for cells found together by ICA. In this case, the spike trains from the image segments are similar but show a progress corresponding to different physiological responses of the cells to the stimulus pattern shown in FIG. 11.

FIG. 15 shows an overlay of the individual filters used to map (and color code) individual cells from the original image.

For individual cells, the sub-cellular details of action potential propagation can be represented by the timing at which an interpolated action potential crosses a threshold at each pixel in the image. Identifying the wavefront propagation may be aided by first processing the data to remove noise, normalize signals, improve SNR, other pre-processing steps, or combinations thereof. Action potential signals may first be processed by removing photobleaching, subtracting a median filtered trace, and isolating data above a noise threshold. The AP wavefront may then be identified using an algorithm based on sub-Nyquist action potential timing such as an algorithm based on the interpolation approach of Foust, et al., 2010, Action potentials initiate in the axon initial segment and propagate through axon collaterals reliably in cerebellar Purkinje neurons. J. Neurosci 30, 6891-6902 and Popovic et al, 2011, The spatio-temporal characteristics of action potential initiation in layer 5 pyramidal neurons: a voltage imaging study. J. Physiol. 589, 4167-4187.

6b. Signal Processing Via Sub-Nyquist Action Potential Timing (SNAPT)

A sub-Nyquist action potential timing (SNAPT) algorithm highlights subcellular timing differences in AP initiation. For example, the algorithm may be applied for neurons expressing Optopatch1, containing a voltage reporter such as QuasAr2 or a suitable variant thereof and a voltage actuator such as CheRiff. Either the soma or a small dendritic region is stimulated via repeated pulses of blue light. The timing and location of the ensuing APs is monitored.

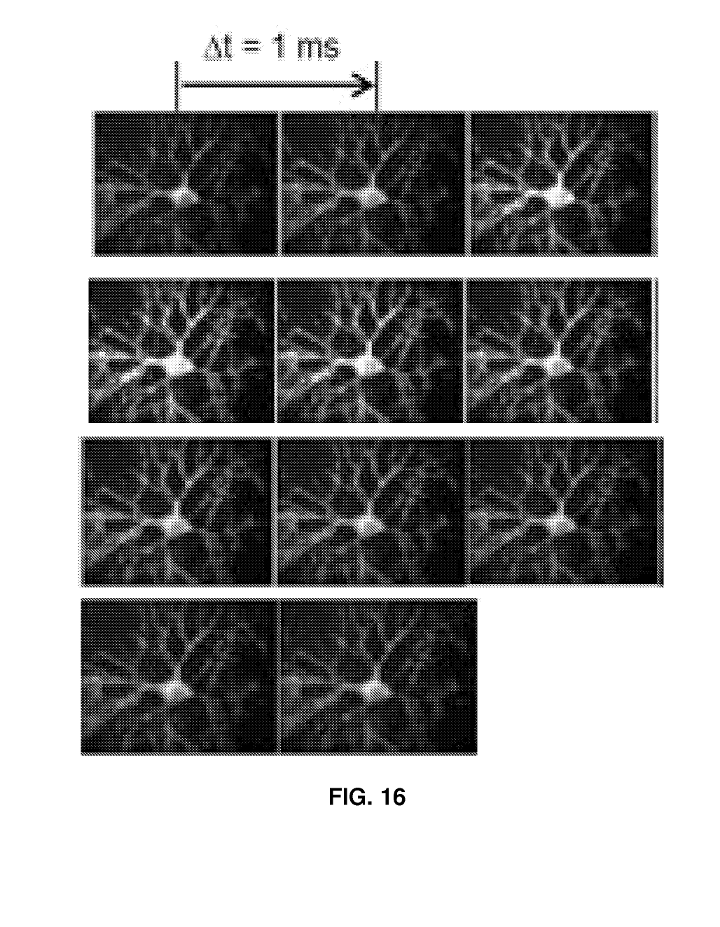

FIG. 16 shows a patterned optical excitation being used to induce action potentials. Movies of individual action potentials are acquired (e.g., at 1,000 frames/s), temporally registered, and averaged.

The first step in the temporal registration of spike movies is to determine the spike times. Determination of spike times is performed iteratively. A simple threshold-and-maximum procedure is applied to the whole-field fluorescence trace, F(t), to determine approximate spike times, {T0}. Waveforms in a brief window bracketing each spike are averaged together to produce a preliminary spike kernel K0(t). A cross-correlation of K0(t) with the original intensity trace F(t) is calculated. Whereas the timing of maxima in F(t) is subject to errors from single-frame noise, the peaks in the cross-correlation, located at times {T}, are a robust measure of spike timing. A movie showing the mean AP propagation may be constructed by averaging movies in brief windows bracketing spike times {T}. Typically 100-300 APs are included in this average. The AP movie has high signal-to-noise ratio. A reference movie of an action potential is thus created by averaging the temporally registered movies (e.g., hundreds of movies) of single APs.

Spatial and temporal linear filters may further decrease the noise in AP movie. A spatial filter may include convolution with a Gaussian kernel, typically with a standard deviation of 1 pixel. A temporal filter may be based upon Principal Components Analysis (PCA) of the set of single-pixel time traces. The time trace at each pixel is expressed in the basis of PCA eigenvectors. Typically the first 5 eigenvectors are sufficient to account for >99% of the pixel-to-pixel variability in AP waveforms, and thus the PCA Eigen-decomposition is truncated after 5 terms. The remaining eigenvectors represented uncorrelated shot noise.

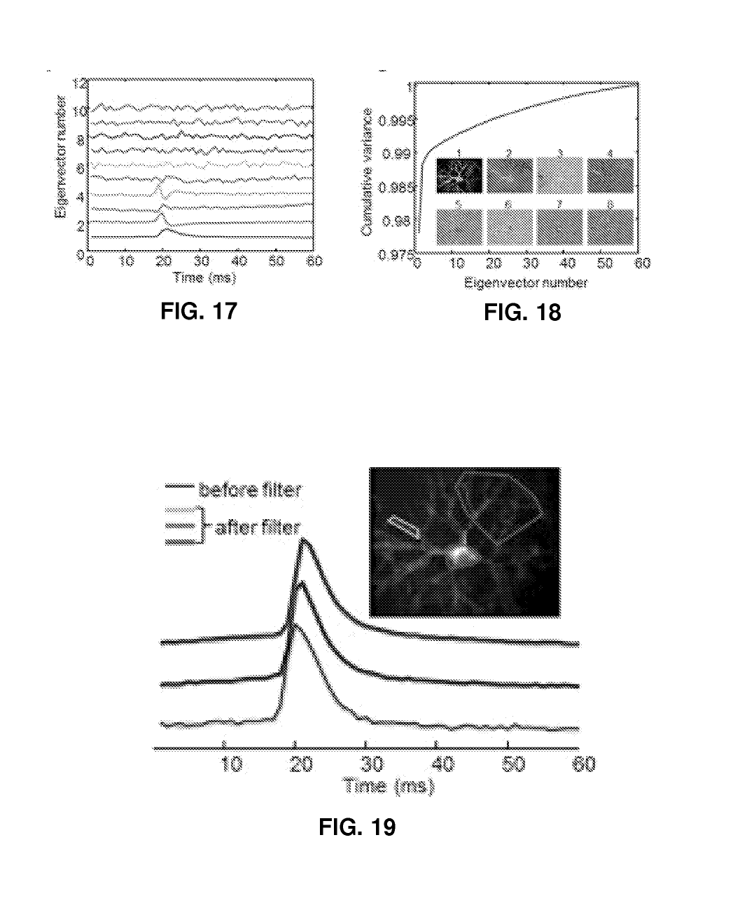

FIG. 17 shows eigenvectors resulting from a principal component analysis (PCA) smoothing operation performed to address noise. Photobleaching or other such non-specific background fluorescence may be addressed by these means.

FIG. 18 shows a relation between cumulative variance and eigenvector number.

FIG. 19 gives a comparison of action potential waveforms before and after the spatial and PCA smoothing operations.

A smoothly varying spline function may be interpolated between the discretely sampled fluorescence measurements at each pixel in this smoothed reference AP movie. The timing at each pixel with which the interpolated AP crosses a user-selected threshold may be inferred with sub-exposure precision. The user sets a threshold depolarization to track (represented as a fraction of the maximum fluorescence transient), and a sign for dV/dt (indicating rising or falling edge. The filtered data is fit with a quadratic spline interpolation and the time of threshold crossing is calculated for each pixel.

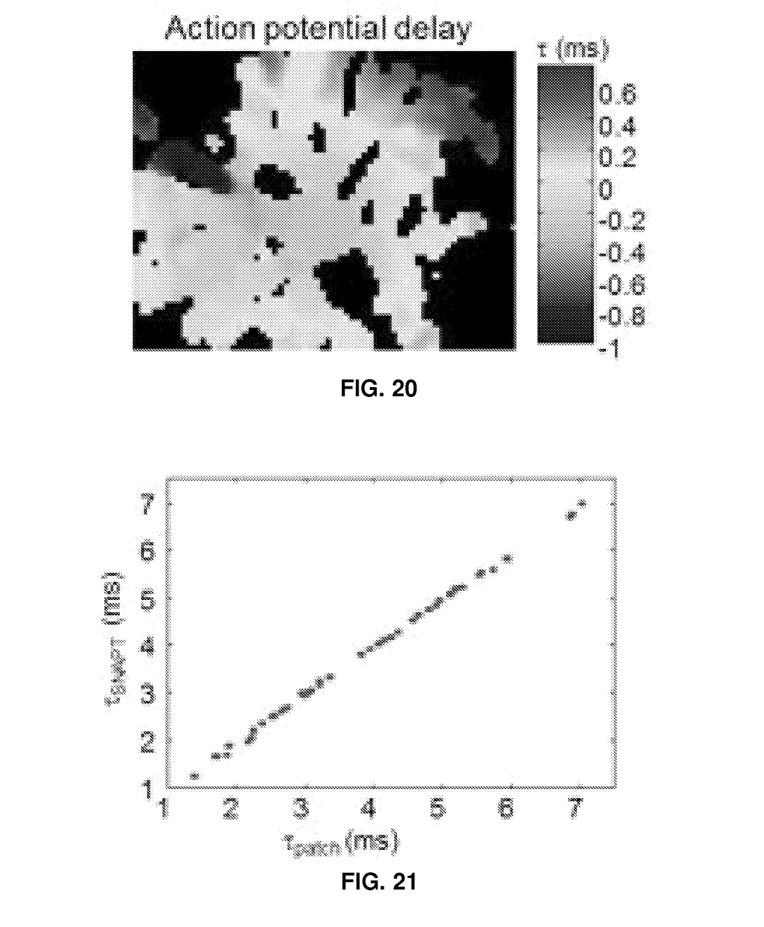

FIG. 20 shows an action potential timing map. The timing map may be converted into a high temporal resolution SNAPT movie by highlighting each pixel in a Gaussian time course centered on the local AP timing. The SNAPT fits are converted into movies showing AP propagation as follows. Each pixel is kept dark except for a brief flash timed to coincide with the timing of the user-selected AP feature at that pixel. The flash followed a Gaussian time-course, with amplitude equal to the local AP amplitude, and duration equal to the cell-average time resolution, σ. Frame times in the SNAPT movies are selected to be ˜2-fold shorter than σ. Converting the timing map into a SNAPT movie is for visualization; propagation information is in the timing map.

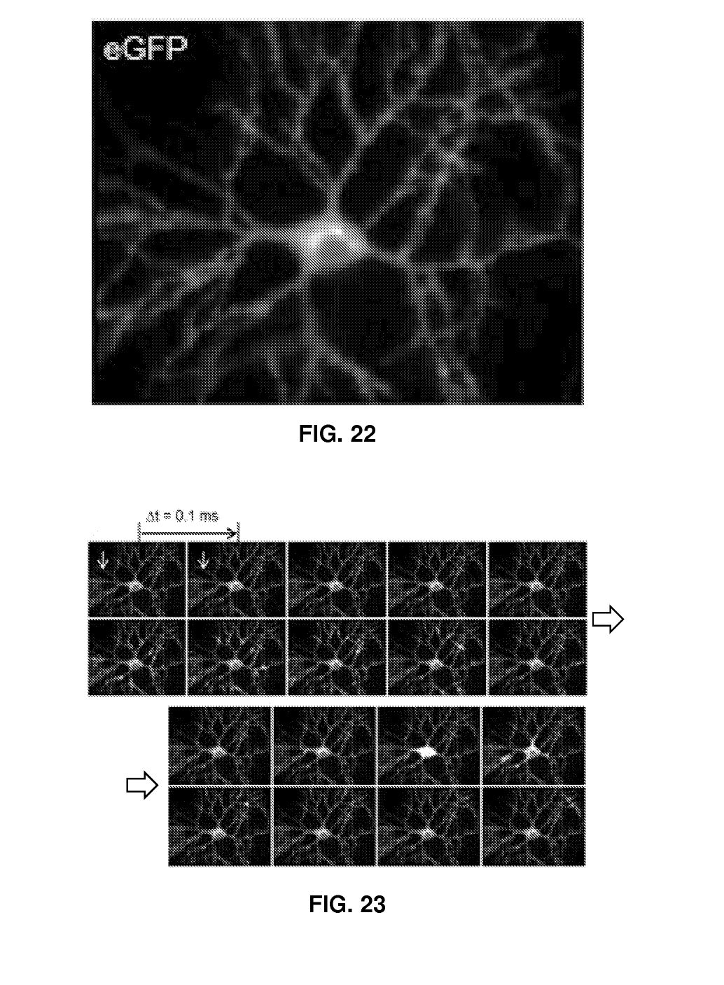

FIG. 21 shows the accuracy of timing extracted by the SNAPT algorithm for voltage at a soma via comparison to a simultaneous patch clamp recording. FIG. 22 gives an image of eGFP fluorescence, indicating CheRiff distribution.

FIG. 23 presents frames from a SNAPT movie formed by mapping the timing information from FIG. 20 onto a high spatial resolution image from FIG. 22. In FIG. 23, the white arrows mark the zone of action potential initiation in the presumed axon initial segment (AIS). FIGS. 20-23 demonstrate that methods of the invention can provide high resolution spatial and temporal signatures of cells expressing an optical reporter of neural activity.

After acquiring Optopatch data, cells may be fixed and stained for ankyrin-G, a marker of the axon initial segment (AIS). Correlation of the SNAPT movies with the immunostaining images establish that the AP initiated at the distal end of the AIS. The SNAPT technique does not rely on an assumed AP waveform; it is compatible with APs that change shape within or between cells.

The SNAPT movies show AP initiation from the soma in single neurites in measured cells. The described methods are useful to reveal latencies between AP initiation at the AIS and arrival in the soma of 320±220 μs, where AP timing is measured at 50% maximum depolarization on the rising edge. Thus Optopatch can resolve functionally significant subcellular details of AP propagation. Discussion of signal processing may be found in Mukamel et al., 2009, Automated analysis of cellular signals from large-scale calcium imaging data, Neuron 63(6):747-760.

Methods of the invention are used to obtain a signature from the observed cell or cells tending to characterize a physiological parameter of the cell. The measured signature can include any suitable electrophysiology parameter such as, for example, activity at baseline, activity under different stimulus strengths, tonic vs. phasic firing patterns, changes in AP waveform, others, or a combination thereof. Measurements can include different modalities, stimulation protocols, or analysis protocols. Exemplarily modalities for measurement include voltage, calcium, ATP, or combinations thereof. Exemplary stimulation protocols can be employed to measure excitability, to measure synaptic transmission, to test the response to modulatory chemicals, others, and combinations thereof. Methods of invention may employ various analysis protocols to measure: spike frequency under different stimulus types, action potential waveform, spiking patterns, resting potential, spike peak amplitude, others, or combinations thereof.

In certain embodiments, the imaging methods are applied to obtain a signature mean probability of spike for cells from a subject and may also be used to obtain a signature from a control line of cells such as a wild-type control (which may be produced by genome editing as described above so that the control and the wild-type are isogenic but for a single site). The observed signature can be compared to a control signature and a difference between the observed signature and the expected signature corresponds to a characteristic of the cell.

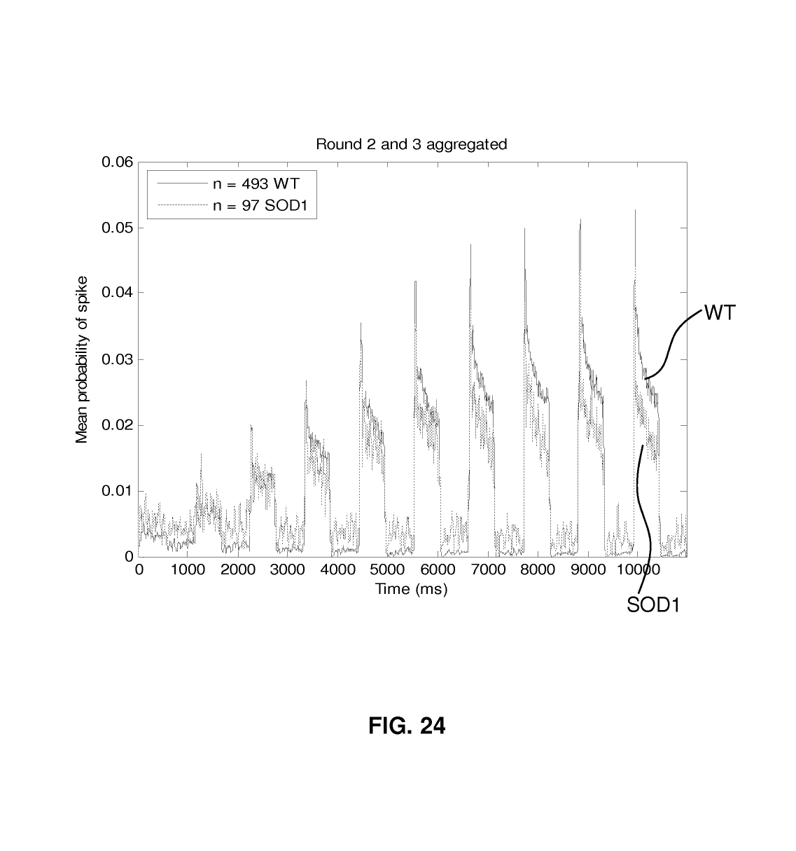

FIG. 24 shows a mean probability of spike of wild-type (WT) and mutant (SOD1 A4V) motor neurons derived from human induced pluripotent stem cells. The SOD1 A4V mutations is associated with amyotrophic lateral sclerosis (ALS). Cellular excitability was measured by probability of spiking during each blue light stimulation, and during no stimulation (spontaneous firing). The mutant neurons had increased rate of firing in the absence of optical stimulation, but a decreased rate of firing under strong optical stimulation.

### 7. Cellular Interactions

In embodiments of the invention, one cell contains the actuator and another cell contains a voltage reporter as well as optionally a calcium reporter (e.g., both within a single fusion protein). In preferred embodiments of the invention, a group of cells contain at least one actuator and another group of cells contain at least one reporter. Both groups of cells are in communication with each other, thereby forming a network. A network, as used herein, relates to a system of at least two cells that are in electrical or chemical communication to each other. Investigations of network effects, as used herein, incorporate this interconnected system of neurons to investigate communication therebetween. The ability to probe network effects may be particularly important as many genes, such as ones that are being implicated in schizophrenia and bipolar disorder, code for synaptic proteins. Network effects also promise to be important in the cardiac area, where for example a monolayer of cardiomyocytes may be illuminated with some cells expressing actuators of the invention while imaged via others expressing the reporters.

Additionally, methods of the invention may be employed to study and use network effects whereby one cell or a genetically specified cell type is stimulated, and a different one is recorded. In addition, methods of the invention allow for investigations into genetics, as many genes, such as ones that are being implicated in schizophrenia and bipolar disorder, code for synaptic proteins. See background discussion neural activity in U.S. Pat. No. 8,401,609 to Deisseroth, the contents of which are incorporated by reference.

In some embodiments, the invention provides a method where in a first set of cells, each cell includes an actuator and in a second set of cells, each cell includes an optical reporter. The method includes stimulating the first set of cells and measuring a signal from the optical reporter in the second set, thereby evaluating whether cells of the first set of cells transmitted a signal to cells of the second set of cells. Preferably, the actuator is an optical actuator such as CheRiff and stimulating the first set of cells includes illuminating the CheRiff actuator.

In some embodiments, the invention provides a method where in a first set of cells, only a few cells include an actuator and in a second set of cells, only a few cells include an optical reporter. The method includes stimulating the first set of cells and measuring a signal from the optical reporters in the second set, thereby evaluating whether cells of the first set of cells transmitted a signal to cells of the second set of cells. Investigations of transmittal length and strength are allowed.

Preferred methods of the invention study the propagation of signals between cells. Methods of the invention may incorporate any type of cell known to accept and/or receive a signal, such as an electrical signal. In addition, propagation of signals between cells may be between like cells, or differing cells.

Preferred methods of the invention involving the characterization of propagation signals between two neurons, between clusters of neurons, or between neurons and other cells. A typical neuron has a cell body, thousands of dendrites, and one axon. Typically, incoming signals are received through the dendrites and the outgoing signal flows along the axon. At the end of the axon are the axon terminals, which contain neurotransmitters. Communication between neurons is achieved at synapses by the process of neurotransmission. Initially, an action potential is generated near the cell body portion of the axon. This signal is then propagated along the axon, away from the dendrites, towards the axon terminals. Conduction ends at the axon terminals. At the axon terminals, electrical synapses, the output is the electrical signal. At chemical synapses, the output is the neurotransmitter.

In vivo, a neuron cannot fulfill its function if it is not connected to other neurons in a network. Therefore, methods of the present invention involve at least two cells. In the simplest form, one cell contains the actuator and the other contains a reporter. The actuator is stimulated and the reporter releases an optically detectable signal is the signal propagated between the neurons. Additionally, it is noted that where networks of cells signal, the signals may propagate from cell to cell in a one-to-one, one-to-many, many-to-one, many-to-many schema, or a combination thereof. That is, axon terminals of two or more neurons may be in synaptic communication with dendrites of one or more other neurons. Where a plurality of cells form a network, signal processing described above may be employed to discern which individual cells have signaled which, when, and how quickly. Thus systems and methods of the invention may be used to—for example—study, discover, or diagnose a condition affecting a synaptic protein.

In preferred embodiments, methods of the present invention may involve a network of neurons (or neural network), where in a group of neurons, information flows from one neuron to another. Preferred methods of the invention characterize the communication between a network of neurons. A neuron containing an actuator is stimulated and releases a signal. A proximate neuron receives the signal as input, processes that signal then sends a signal as output to other neurons through synapses. A downstream neuron receiving a signal contains a reporter. Upon the neuron receiving a signal from another neuron in the network, the reporter releases an optically detectable signal.

Network effects are also important in the cardiac area, where for example, a monolayer of cardiomyocytes may be illuminated with some cells expressing actuators of the invention while imaged via others expressing the reporters. Cells of the invention (e.g., neurons, cardiomyocytes, etc.) may be visualized via a microscope of the invention. Those cells may be in electrical or synaptic communication with one another.

7a. In Vitro Agent Screening

The present invention allows for the optical detection of electrophysiological indicators of cells containing the optogenetic actuators and/or reporters of the invention. In methods of the invention, a cell or a set of cells contains an actuator and a different cell or a set of cells contains the reporter. Upon receiving a signal from another cell, a cell containing a reporter may be caused to express an optical reporter of neural or electrical activity. In some methods of the invention, cells, or a network of cells, may be exposed to an agent, such as a drug. In some methods of invention, the exterior of the cells are exposed to an agent, for example nanoparticles. A signature that characterized the cells or network of cells can be observed before, during, or after testing the compound. Any combination of different cells and cell types can be exposed to one or any combination of compounds, including different test compound controls.

In certain embodiments of the invention, various cells may be incorporated into the assays and processes. Cells can be derived from various sources, as discussed above, and cells can be derived from various organs of a specimen. For example, cells from lung tissue, kidney tissue, pancreatic tissue, stomach tissue, dermal tissue, gall bladder tissue, cardiac tissue, muscle tissue, etc. can be incorporated into assays of the invention. Multiple cell types can be incorporated into assays and methods of invention to efficiently and comprehensively determine the effects a single agent or a group of agents have on cellular signatures or characteristics. Furthermore, in some embodiments, only some cell types include the optogenetic actuators and/or reporters of the invention.

Cells can be divided into multiple subsets. Forming cell subsets (particularly microscopic cell subsets) is useful for sampling multiple tissue culture conditions as each cell subset constitutes a unit that can be exposed to a variety of cell culture conditions and agents. For example, the subsets can be arranged in arrays and then exposed to various agents, using the principles of combinatorial chemistry. Arrangements in arrays allow for a plurality of agents to be investigated in parallel, accomplishing many assays simultaneously. A set of subsets may comprise 2, 4, 10, several hundred or several thousand subsets. Each subset may comprise 1, 50, 390, 800, several hundred or several thousand cells.

In embodiments using subsets, a single subset can be engineered and designed to represent a network. For example, a subset may include two types of cells: cells containing actuators and cells containing reporters. In a preferred embodiment, for example, a subset would contain a network of neurons or cardiac cells. The cells are arranged within the subset to allow for electrical or chemical signals to be propagated when the actuator is activated. If a signal successfully propagates, an optically detectable signal is detected. Therefore, an array of multiple subsets allows for the investigation of network effect of a plurality of agents.

In some embodiments of the invention, cells, or subsets of cells may be labeled. A label or tag may be used to identify a cell or a cell subset to determine a culture condition, a sequence of culture conditions, an agent exposed to a cell subset, or agents exposed to a cell subset. A single label or multiple labels may be used to identify a cell or cell subset. A label or multiple labels may be added at a specific culturing step, when an agent or plurality of agents is added to a subset, or added as a positional reference. Examples of labels or tags are molecules of unique sequence, structure or mass; or fluorescent molecules or objects such as beads; or radiofrequency and other transponders; or objects with unique markings or shapes. Labelling of cell units may be achieved by a variety of means, for instance labelling either the cells themselves, or any material to which the cells are attached or otherwise associated with. Any of the chemical and non-chemical methods used to encode synthetic combinatorial libraries can be adapted for this purpose and some of these are described in Methods in Enzymology Vol 267 (1996), ‘Combinatorial Chemistry’, John N. Abelson (Editor); Combinatorial Chemistry, Oxford University Press (2000), Hicham Fenniri (Editor); K. Braeckmans et al., ‘Scanning the code’, Modern Drug Discovery (February 2003); and Braeckmans et al., 2002, Encoding microcarriers: Present and Future Technologies, Nature Reviews Drug Discovery, 1::447-456 (2002), all of which are herein incorporated by reference. Detection of tags can be accomplished by a variety of methods familiar to those skilled in the art. Methods include mass spectrometry, nuclear magnetic resonance, sequencing, hybridisation, antigen detection, electrophoresis, spectroscopy, microscopy, image analysis, fluorescence detection, etc.

In some embodiments, combinatorial cell culture or split-pool cell culture may be used, which involves the serial subdividing and combining of subsets in order to sample multiple combinations of cell culture conditions or exposure to agents. See for example, United States Patent Publication 2007/0298411, the contents of which are incorporated by reference. An initial starter culture (or different starter cultures) of cell subsets are divided into x number of aliquots which are grown separately under different culture conditions or are exposed to various differing agents. Following cell culture for a given time, the cell subsets can be pooled by combining and mixing the different aliquots. This pool can be split again into x2 number of aliquots, each of which is cultured under different conditions for a period of time, and subsequently also pooled. This iterative procedure of splitting, culturing and pooling (or pooling, splitting and culturing; depending on where one enters the cycle) cell units allows systematic sampling of many different combinations of cell culture conditions.

In some embodiments, splitting and/or pooling cell subsets are according to a predetermined protocol, the overall effect being that adventitious duplications or omissions of combinations are prevented. Predetermined handling of cell subsets can be optionally planned in advance and logged on a spreadsheet or computer program, and splitting and/or pooling operations executed using automated protocols, for instance robotics. Robotic devices capable of determining the identity of a sample, and therefore partitioning the samples according to a predetermined protocol, have been described (see ‘Combinatorial Chemistry, A practical Approach’, Oxford University Press (2000), Ed H. Fenniri). Alternatively, standard laboratory liquid handling and/or tissue culture robotics (for example such as manufactured by: Beckman Coulter Inc, Fullerton, Calif.; The Automation Partnership, Royston, UK) is capable of spatially encoding the identity of multiple samples and of adding, removing or translocating these according to pre-programmed protocols.

In some embodiments of the invention, a cell subset is spatially resolved on a substrate. In some embodiments of the invention, a cell subset is contained within well, or small, open divot. In some embodiments of the invention, a cell subset is contained within a closed well, or small, closed divot. In some embodiments of the invention, a cell or a cell subset is contained within a well or a multi-well plate or substrate. The multi-well plate or substrate may contain 96, 384, 1536, or 3456 wells. In some embodiments of the invention, a cell subset is contained within a vessel, where a vessel is construed as any means suitable to contain a cell culture. A vessel may be a petri-dish; a cell culture bottle or flask; or a multi-well plate.

In some embodiments of the invention, the assay may be of a short duration or a long duration. For example, to prepare for an assay, each well or vessel may be filled with a cell subset. An agent or agents may be added to the well. In some embodiments, a label may be added. After some incubation time has passed to allow the biological matter to absorb, bind to, or otherwise react (or fail to react) with the agent or agents in the wells, measurements are taken across all the plate's wells, either manually or by a machine. The time period for a first measurement may be under a minute, after a few minutes, after an hour, after several hours, or after a few days. It should be appreciated that when measurements are taken during a time period is dependent upon the protocol used, the agent or agents being investigated, and the assay being used. In addition, measurements may be taken at any interval over a defined time period. For example, hourly measurements may be taken over the course of a day, several days, or several weeks. Measurements may be taken over the course of several weeks. Measurements may be taken over the course of several months. It should be appreciated that any time period may be defined within the protocol of in vitro drug assay.

In some embodiments, controls may be incorporated, whether positive or negative, or both. Negative controls are subsets where no change is expected. Positive controls are subsets where a change is expected. For example, in assays of multiple subsets, one or more subsets can be treated to serve as a negative control, while another subset or group of subsets are treated to serve as a positive control. In addition, some assays may only take an initial measurement to serve as a base-line measurement and subsequent measurements are compared to the base-line measurement. Assays may include blind and double-blind protocols.

Methods of the invention include in vitro assay screening of various agents and conditions across varying subsets. Cells, uniform or varying in type, are exposed to one or multiple agents to comprehensively understand the cellular response from an agent. A single agent may be applied to a cell subset, or multiple agents may be applied to a cell subset. Methods of the invention may include any agent or any combination of agents. An agent may be a drug, a compound, a protein, an element, a nucleic acid, a virus, a pathogen, an enzyme, antibodies, genes, nanoparticles, etc.

Methods of the invention may include in vitro drug assays to detect the electrophysiological effects on a cell after the application of a compound and this can be used to detect and eliminate unsuccessful drug candidates prior to clinical trials. For example, the methods allow for determining which drug candidates impact membrane potentials by measuring membrane potentials before, during and after exposure to a drug compound. For example, the methods allow for determining which drug candidates impact signal propagation by characterizing detectable signals before, during and after exposure to a drug compound. Application of compounds can reveal the effects of those compounds on cellular electrophysiology and firing sequences of neurons, cardiomyocytes, or pacemaker cells. The measured signature can include any suitable electrophysiology parameter such as, for example, activity at baseline, activity under different stimulus strengths, tonic vs. phasic firing patterns, changes in AP waveform, others, or a combination thereof. Measurements can include different modalities, stimulation protocols, or analysis protocols. Exemplarily modalities for measurement include voltage, calcium, ATP, or combinations thereof. Exemplary stimulation protocols can be employed to measure excitability, to measure synaptic transmission, to test the response to modulatory chemicals, others, and combinations thereof. Methods of invention may employ various analysis protocols to measure: spike frequency under different stimulus types, action potential waveform, spiking patterns, resting potential, spike peak amplitude, others, or combinations thereof.

In some embodiments of the invention, the principles of combinatorial chemistry are utilized to aid in the drug development process. Combinatorial chemistry comprises chemical synthetic methods that make it possible to prepare a large number (tens to thousands or even millions) of compounds in a single process. These compound libraries can be made as mixtures, sets of individual compounds or chemical structures generated in silico. The compounds are then screened and analyzed using the methods of the invention.

Principles of combinatorial science may be utilized to screen and test a large number of compounds. A library of different or related compounds may be grouped into a library to determine an agent's effect on cell-cell communication. For example, a subset or multiple subsets may be exposed to various combinations of drugs known to be used by a population of patients to determine possible drug interferences. For example, a drug can be screened with a known over the counter drug to determine any possible interferences or interaction. A drug could be screen in combination with other known drugs a patient afflicted with a known disease may be prescribed. Libraries of known compounds, ions, or elements can be tested in combination to determine the effects, if any, on cell or subsets signature or characterization.

These early-stage in vitro cell-based assays represent essential aspects of in vivo pharmacology and toxicology. The optogenetic actuators and reporters disclosed in the present application can be used in methods for drug screening, e.g., for drugs targeting the nervous system or the circulatory system (cardiomyocytes). In a culture of cells expressing specific ion channels, one can screen for agonists or antagonists without the labor of applying traditional patch clamps or electrodes to cells one at a time. In neuronal cultures one can probe the effects of drugs on action potential initiation, propagation, and synaptic transmission. Application in human iPSC-derived neurons will enable studies on genetically determined neurological diseases, as well as studies on the response to environmental stresses (e.g. anoxia).

Any compound or drug may be used in the methods of the present invention. The present invention provides an in vitro methodology for investigating electrophysiological changes in cells from exposure to a compound. Any drug, molecule, compound, inhibitor, enzyme, or chemical species may be used in conjunction with the present invention. The word drug herein refers to any moiety, which may be an organic or inorganic molecule, a protein, peptide or polypeptide, a hormone, a fatty acid, a nucleotide, a polysaccharide, a plant extract, whether isolated or synthetically produced, etc. which is known to have a beneficial therapeutic activity, when administered to a subject, and includes drugs that are used to treat cardiovascular and neurological diseases, as well as cancer, diabetes, asthma, allergies, inflammation, infections and liver disease.

Methods of the invention may screen for drugs or agents that impact synaptic transmission, for example increasing or decreasing neurotransmitters. It is known in the art that drugs can increase or decrease the effects of neurotransmitters. A drug that works against or blocks the effects of a neurotransmitter is defined as an antagonist. A drug that increases or pushes the effects is defined as an agonist. Some drugs can be both. This type of drug is called a mixed agonist-antagonist, and usually depends on the dose. A drug can decrease or increase the synthesis of the neurotransmitter or even cause it to leak at its vesicles. It can increase its release, block its breaking down process, decrease its reuptake, inactivate chemicals, or even stimulate or stop the postsynaptic receptors.

Methods of the inventions allow for investigations related to synaptic transmissions. For example, neurotoxins interfere with synaptic transmissions by inhibiting neurotransmitter release. For example, the tetanus and botulinum toxins are respectively produced by the bacteria Clostridium tetani and Clostridium botulinum, can cause conditions such as lockjaw. However, these toxins have their greatest effect on inhibition of synaptic transmission by inhibitory neurons in the spinal cord, neurons that would normally inhibit muscle contraction. C. botulinum causes botulism, which is characterized by weakness and paralysis of skeletal muscle as well as by a variety of symptoms that are related to inhibition of cholinergic nerve endings in the autonomic nervous system. In addition, botulinum neurotoxin (BoNT) has recently emerged as a potential novel approach to control pain. Studies have revealed a number of mechanisms by which BoNTs can influence and alleviate chronic pain, including inhibition of pain peptide release from nerve terminals and sensory ganglia, anti-inflammatory and anti-glutaminergic effects, reduction of sympathetic neural discharge, and inhibition of muscle spindle discharge.

Additionally, synapse loss is an early and invariant feature of Alzheimer's disease (AD) and there is a strong correlation between the extent of synapse loss and the severity of dementia. Accordingly, it has been proposed that synapse loss underlies the memory impairment evident in the early phase of AD and that since plasticity is important for neuronal viability, persistent disruption of plasticity may account for the frank cell loss typical of later phases of the disease.

Methods of the invention can be used to screen potential drug candidates for possible treatment of neurotoxins, Alzheimer's, dementia, epilepsy, etc. See for example, Pacico et al., 2014, New in vitro phenotypic assay for epilepsy: fluorescent measurement of synchronized neuronal calcium oscillations, PLoS One 9(1):e84755, which is incorporated by reference, in which cultures were observed at different stages of development (6-20 days in vitro) and at different densities by monitoring their activity over 10-minute periods with data acquisition every 0.8 seconds to investigate the spontaneous development of intracellular calcium oscillations. Investigations were conducted in high density neuronal cultures (50,000 cells per well in 96-well plates). In an aspect of the invention, cells grown in culture can be exposed to a compound and then analyzed at various time points. A cell culture in which cells have been exposed to a particular compound can be analyzed by testing aliquots of the cell culture. The aliquots are exposed to stimulation and analyzed for release of a detectable signal. The aliquot may be cultured for a set time period and again analyzed for release of a detectable signal. The measurements may be repeated for an unlimited number of times.

Employing the methods and applications of the present invention, neuronal properties and cellular integrative mechanisms are investigated without outside influences (e.g., from other neurons or hormones, etc.). In addition, in vitro assays provides a controlled system in which the ability to use known concentrations of drugs allows for the mimicking of the concentrations of neurotransmitters released at the synapse, adjusting applied drug concentrations to those known to exist in blood or brains of the test subject with systemically administered drugs, or using drug concentrations within the known range for selective action at target receptors.

In certain embodiments, compounds are added to cells or subsets and their effect on the cells is observed to distinguish possible diseases or causes or mechanisms of diseases. For example, where two or more cells in synaptic connection with one another are observed, extrinsic stimulation of an upstream cell should manifest as an action potential in a downstream cell. A compound that is known to inhibit neurotransmitter reuptake may be revealed to work on only certain neural subtypes thus indicating a specific disease pattern.

In other embodiments, effects on particular channels may be investigated. The γ-aminobutyric acid A (GABA_{A}) ion channels are important drug targets for treatment of neurological and psychiatric disorders. Finding GABA_{A} channel subtype selective allosteric modulators could lead to new improved treatments. However, the progress in this area has been obstructed by the challenging task of developing functional assays to support screening efforts and the generation of cells expressing functional GABA_{A} ion channels with the desired subtype composition. Assays of the present invention may be utilized to investigate a drug's effect on particular ion channels.

In some embodiments, methods of the invention are used to detect, measure, or evaluate synaptic transmission. A signature may be observed for a cell other than the cell to which direct stimulation was applied. In fact, using the signal processing algorithms, synaptic transmission among a plurality of cells may be detected thus revealing patterns of neural connection. Establishing an assay that successfully detects firing of a downstream neuron upon stimulation of an upstream neuron can reveal, where the subject cell to be observed fails to fire upon stimulation of an upstream neuron, a disease or condition characterized by a failure of synaptic transmission.

In some embodiments, compounds can be evaluated as candidate therapies to determine suitability of a treatment prior to application to patient. For example, one can test epilepsy drugs to find the one that reverts the firing pattern back to wild-type. In some embodiments, the invention provides systems and methods for identifying possible therapies for a patient by testing compounds, which systems and methods may be employed as personalized medicine. Due to the nature of the assays described herein, it may be possible to evaluate the effects of candidate therapeutic compounds on a per-patient basis thus providing a tool for truly personalized medicine. For example, an assay as described herein may reveal that a patient suffering from a certain disease has neurons or neural subtypes that exhibit a disease-type physiological phenotype under the assays described herein. One or a number of different compounds may be applied to those neurons or neural subtypes. Cells that are exposed to one of those different compounds (or a combination of compounds) may exhibit a change in physiological phenotype from disease-type to normal. The compound or combination of compounds that affects the change in phenotype from disease-type to normal is thus identified as a candidate treatment compound for that patient.

Similarly, the optical voltage sensing using the constructs provided herein provides new and much improved methods to screen for drugs that modulate the cardiac action potential and its intercellular propagation. These screens will be useful both for determining safety of candidate drugs and to identify new cardiac drug leads. Identifying drugs that interact with the hERG channel is a particularly promising direction because inhibition of hERG is associated with ventricular fibrillation in patients with long QT syndrome. Application in human iPSC-derived cardiomyocytes will enable studies on genetically determined cardiac conditions, as well as studies on the response to environmental stresses (e.g. anoxia).

Methods of the invention may incorporate the use of statistical methods, common in the field. Calculations such as z-scores, t-tests, mean difference, percent inhibition, percent activity, outlier, B-score method, and quantile-based methods may be utilized for analysis.

### Types of Assays Using Network Effects

Aspects of the invention may be utilized in various assays during the drug development and drug screening processes. It should be appreciated that the optogenetic actuators and reporters of the invention may be incorporated into all cells, or a selected number of cells within an assay. Other detection methods may be utilized in combination with the optical detection methods of the invention.

In some embodiments of the invention, assays such as enzymatic assays, inflammatory assays, signaling assays, uptake assays and proliferation assays are used in conjunction with the invention.

In some embodiments, the cells of the network may not be exposed to an agent; however, the cell environment is exposed to an agent. Exposure to nanoparticles in vivo increases the risk of onset of neurodegenerative diseases and nanoparticles are apparently able to kill neurons in vitro. See for example Fedorovich, et al., Are synapses targets of nanoparticles?, Biochem. Soc. Trans., 2010 April; 38(2):536-8. Therefore, the cellular environment may be exposed to an agent, such as a nanoparticle, to determine the network effects of nanoparticles in the space surrounding the cells.

In some embodiments, several drugs may be screened on the same subset to determine possible interactions. One drug might inhibit the metabolism of a co-administered drug. The affected drug thereby might have plasma concentrations, higher than intended, leading to toxicity. A well-known case of inhibitory drug-drug interactions is the inhibition of the metabolism of a non-drowsy antihistamine, terfenadine, by the antifungal drug, ketoconazole. A number of patients developed fatal cardiac arrhythmia after they were administered both terfenadine and ketoconazole owing to the elevated level of terfenadine. Terfenadine has been taken off the market owing to its drug-drug interaction potential. It is now known that ketoconazole is a potent inhibitor of CYP3A4, the P450 isoform responsible for metabolism of terfenadine. A drug can also accelerate the metabolism of a co-administered drug by inducing the corresponding metabolic pathways. In this situation, the major result is the diminished efficacy of the affected drug owing to the lower-than-intended plasma level. Rifampin-birth control pill interaction is an example of drug-drug interactions. Women of child-bearing age on birth control pill experienced pregnancy owing to the induction of the drug-metabolizing enzymes for the active ingredients of the birth control pills. Rifampin is now known to induce two major pathways of the metabolism of birth control pill ingredients: CYP3A4 and estrogen sulfotransferase.

In some embodiments of the invention, application of agents or compounds can be used to define or characterize the effect of an agent on cell toxicity. Accurate prediction of drug safety remains the major challenge for the pharmaceutical industry. Early screening for drug toxicity, especially human-specific toxicity, has become a routine practice in drug discovery and development. The use of human cells, which retains organ-specific properties represent important experimental systems for early toxicity screening. The promising primary human cell culture systems include the following: hepatocytes for liver toxicity; renal proximal tubule epithelial cells for nephrotoxicity; vascular endothelial cells for vascular toxicity; neuronal and glial cells for neurotoxicity; cardiomyocytes for cardiotoxicity; and skeletal myocytes for rhadomyolysis.

Cytotoxicity is screened using parameters such as membrane integrity, cellular metabolite content, mitochondrial functions and lysosomal functions. Membrane integrity is the measurement of the increase in cytoplasmic enzymes such as lactate dehydrogenase in the culture medium after treatment. This endpoint represents a classical endpoint for cytotoxicity. For some cell types (e.g. hepatocytes), the basal level can be too high and therefore might limit the sensitivity of the endpoint. Cellular metabolite content represents the most commonly measured metabolite content. Dead and damaged cells contain little or no ATP. Bioluminesence assay using the luciferin-luciferase assay represent a sensitive assay for cellular ATP, allowing the use of as little as a few hundred cells per assay. To determine mitochondrial functions, the chemical, 3-(4,5-dimethylthiazol-2-yl)-2,5-diphenyltetrazolium bromide (MTT), is converted to a blue crystal which can be solubilized and quantified by spectrophotometry. The MTT assay is a common assay for cytotoxicity and usually would yield results similar to the ATP content assay. To measure lysosomal functions, neutral red uptake assay is used to measured cell viability as reflected by lysosomal functions. Neutral red is taken into a cell owing to lysosomal activities. Cell damage would be accompanied by a decreased neutral red uptake. The assay involves incubation of cells with neutral red, followed by washing of extracellular dye, and quantification of uptake by cell solubilization and spectrometry. Apoptosis is measured by the induction of programmed cell death or apoptosis is a known mechanism of drug toxicity. There are also specific systems for the evaluation of toxicity of known mechanism. For instance, QT prolongation, a manifestation of cardiac arrhythmia, can be studied in cardiomyocytes.

In some embodiments of the invention, Integrated Discrete Multiple Organ Co-culture (IdMOC) processes are incorporated to effectively characterize an agent or a group of agents. In an IdMOC system of the invention, cells from different organs are physically separated but interconnected and are co-cultured. The system uses a ‘wells-within-a-well’ concept for the co-culturing of cells or tissue slices from different organs as physically separated (discrete) entities in the small inner wells. These inner wells are nevertheless interconnected (integrated) by overlying culture medium in the large outer containing well. The IdMOC system thereby models the in vivo situation, in which multiple organs are physically separated but interconnected by the systemic circulation, permitting multiple organ interactions. The IdMOC system, with either cells or tissue slices from multiple organs, can be used to evaluate cell type-specific or organ-specific toxicity.

In some embodiments of the present invention, an agent or a group of agents are exposed to cells within an IdMOC system. The interconnectedness of the cells allows for various types of cells to be investigated simultaneously to capture the full effectiveness of an agent or group of agents. In the IdMOC system, the optogenetic actuators or reporters may be incorporated into each cell, or only a select number of cells. Cells within the system may be analyzed by the optical detection methods of the invention, or in combination with other detection methods.

A major drawback of in vitro system is that each cell type is studied in isolation, whereas in the human body, there might be multiple organ interactions that are critical to drug toxicity. For instance, a drug can be first metabolized by the liver and the liver metabolites can cause toxicity in a distant organ. To overcome this deficiency of single cell type in vitro systems, the independent discrete multiple organ co-culture (IdMOC) system, has been developed. The IdMOC allows the co-culturing of cells from different organs as physically separated cultures that are interconnected by an overlying medium, akin to the blood circulation connecting the multiple organs in the human body.

The IdMOC models the multiple organ interaction in human in vivo, allowing the evaluation of organ-specific effect of a drug and its metabolites. The IdMOC consists of multiple wells (inner wells) within a larger well (containing chamber). Multiple cell types are firstly individually cultured in the inner wells in media optimized for each cell type). On the day of experimentation, the individual media are removed and the chamber will be filled with a single medium, flooding all the inner wells, thereby allowing well-to-well communication via the overlying medium. The test materials are added to the overlying medium. After experimentation, the overlying medium can be analyzed for overall metabolism of the test material and the individual cell types can be processed for the quantification of associated test material to evaluate possible organ-specific bioaccumulation, evaluation of cell viability for cytotoxicity, and evaluation of efficacy.

In some embodiments of the invention, the principles of Hit to Lead (H2L) are applied to optimize or fully characterize an agent with several rounds of assays. Hit to lead (H2L) also known as lead generation is a stage in early drug discovery where small molecule hits from a high throughput screen (HTS) are evaluated and undergo limited optimization to identify promising lead compounds. Drug discovery processes may involve the identification of screening hits, medicinal chemistry and optimization of those hits to increase the affinity, selectivity (to reduce the potential of side effects), efficacy/potency, metabolic stability (to increase the half-life), and oral bioavailability. Once a compound that fulfills all of these requirements has been identified, it will begin the process of drug development prior to clinical trials. The lead compounds undergo more extensive optimization in a subsequent step of drug discovery called lead optimization (LO). After hits are identified from a high throughput screen, the hits are confirmed and evaluated using re-testing, dose response curve generation, orthogonal testing secondary screening, chemical amenability, biophysical testing, and hit ranking and clustering.

Methods of the invention may involve arranging multiple subsets of cells on a substrate. One or more subsets contain a network, where cells, such as neurons, are arranged to be in synaptic communication. However, only some cells contain actuators and others contain reporters, creating the network described above. Other subsets include various other cells which may contain both actuators and reporters. The other cells are from various organs, such as the liver, pancreas, gall bladder, etc. The subsets are exposed to stimuli and detectable signals are recorded. The plurality of subsets is exposed to an agent, or a combination of agents. The subsets are exposed to stimuli and detectable signals are recorded. Evaluation of the two signals allows for characterization of the agent on the various subsets, allowing for a comprehensive analysis of the agent. It should be appreciated that this example is only demonstrative; other combinations of agents, subsets, measurement parameters, etc. can be employed.

In some embodiments, the cells are genetically modified. At least two genetically modified cells are placed within communication of each other; wherein one contains an actuator and one contains a reporter. The cells are investigated for the genetic effects of sending or receiving signals between cells. In other embodiments, genetically modified cells are incorporated into a network.

Methods of the inventions allow for investigations related to ion channels. A recent article reported that among the 100 top-selling drugs, 15 are ion-channel modulators with a total market value of more than $15 billion. See Molokanova & Savchenko, 2008, A. Drug Discov. Today 13:14-22. However, searches for new ion-channel modulators are limited by the absence of good indicators of membrane potential. Ion channels are important drug targets because they play a crucial role in controlling a very wide spectrum of physiological processes and because their dysfunction can lead to pathophysiology. New generations of therapeutic agents are expected to result from discovering and commercializing successful drugs that modulate the activity of voltage-gated sodium, calcium, or potassium channels, or ligand-gated ion channels. Electrophysiology pertains to the flow of ions in biological tissues and, in particular, to the measuring of this flow. At the cellular level, electrical activity of neurons consists of the movement of charges (ions) through neuronal surface membranes. The major charge carrying ions are sodium (Na⁺), potassium (K⁺), chloride (Cl⁻) and calcium (Ca²⁺). The surface membranes of neurons are primarily composed of lipids (resistive elements, in electrical terms) which do not allow ionic flow. Instead, these semipermeable membranes are spanned by large specialized protein aggregates that form pores or channels through the lipid membrane. There are specific channel protein assemblies (usually more than one) for each of the ionic charge carriers, as well as those for certain cations in general, that confer a semipermeable nature to the membrane. The ability of these channels to permit ion flow is determined by several factors, most prominently the electrical potential that exists across the membrane, the gradient of ions set up by membrane pumps, and the semipermeable nature of the channels, as well as by responses of receptors, guanosine triphosphate (GTP) binding proteins (termed G proteins), and second messengers to neurotransmitters and hormones.

In some embodiments of the invention, a compound effect on an ion channel can be characterized. For example, for determining compounds that effect calcium oscillations, methods of invention can be employed to systematically determine which compounds result in the desired effects prior to pre-clinical investigations. Calcium signaling results from a complex interplay between activation and inactivation of intracellular and extracellular calcium permeable channels. In excitable cells, such as the heart for example, these may be comprised of, or initiated by regenerative all-or-none plasma membrane channel activation, the Ca²⁺ action potential with amplification by intracellular Ca²⁺ release. Calcium oscillations can be investigated using cell cultures of cortical and hippocampal primary cultures from mouse or rat tissue. See for example, Pacico et al., 2014, New in vitro phenotypic assay for epilepsy: fluorescent measurement of synchronized neuronal calcium oscillations, PLoS One 9(1):e84755, which is incorporated by reference, in which cultures were observed at different stages of development (6-20 days in vitro) and at different densities by monitoring their activity over 10-minute periods with data acquisition every 0.8 seconds to investigate the spontaneous development of intracellular calcium oscillations. Investigations were conducted in high density neuronal cultures (50,000 cells per well in 96-well plates). Cells grown in culture can be exposed to a compound and then analyzed at various time points. A cell culture in which cells have been exposed to a particular compound can be analyzed by testing aliquots of the cell culture. The aliquots are exposed to stimulation and analyzed for release of a detectable signal. The aliquot may be cultured for a set time period and again analyzed for release of a detectable signal. The measurements may be repeated for an unlimited number of times.

Employing the methods and applications of the present invention, neuronal properties and cellular integrative mechanisms are investigated without outside influences (e.g., from other neurons or hormones, etc.). In addition, in vitro assays provides a controlled system in which the ability to use known concentrations of drugs allows for the mimicking of the concentrations of neurotransmitters released at a synapse, adjusting applied drug concentrations to those known to exist in blood or brains of the test subject with systemically administered drugs, or using drug concentrations within the known range for selective action at target receptors. In some embodiments, the optical reporters described herein are used to measure or monitor membrane potential changes in response to a candidate ion channel modulator. Such screening methods can be performed in a high throughput manner by simultaneously screening multiple candidate ion channel modulators in cells.

7b. Multimodal Sensing/Multiplexing

Membrane potential is only one of several mechanisms of signaling within cells. One may correlate changes in membrane potential with changes in concentration of other species, such as Ca++, H+ (i.e. pH), Na+, ATP, cAMP. We constructed fusions of Arch with pHluorin (a fluorescent pH indicator) and GCaMP6f (a fluorescent Ca++ indicator). One can also use fusions with other protein-based fluorescent indicators to enable other forms of multimodal imaging using the concept as taught herein. Concentration of ions such as sodium, potassium, chloride, and calcium can be simultaneously measured when the nucleic acid encoding the microbial rhodopsin is operably linked to or fused with an additional fluorescent ion sensitive indicator.

Additional fluorescent proteins may be included. The term “additional fluorescent molecule” refers to fluorescent proteins other than microbial rhodopsins. Such molecules may include, e.g., green fluorescent proteins and their homologs.

Fluorescent proteins that are not microbial rhodopsins are well known and commonly used, and examples can be found, e.g., in a review Wachter, 2006, The Family of GFP-Like Proteins: Structure, Function, Photophysics and Biosensor Applications. Introduction and Perspective, Photochem and Photobiol 82(2):339-344. Also, Shaner et al., 2005, A guide to choosing fluorescent proteins, Nat Meth 2:905-909 provides examples of additional useful fluorescent proteins.

One can combine imaging of voltage indicating proteins with other structural and functional imaging, of e.g. pH, calcium, or ATP. One may also combine imaging of voltage indicating proteins with optogenetic control of membrane potential using e.g. channelrhodopsin, halorhodopsin, and Archaerhodopsin. If optical measurement and control are combined in a feedback loop, one can perform all-optical patch clamp to probe the dynamic electrical response of any membrane.

The invention provides high-throughput methods of characterizing cells. Robotics and custom software may be used for screening large libraries or large numbers of conditions which are typically encountered in high throughput drug screening methods.

### 8. Measurement Methodologies

The spectroscopic states of microbial rhodopsins are typically classified by their absorption spectrum. However, in some cases there is insufficient protein in a single cell to detect spectral shifts via absorbance alone. Any of the following several optical imaging techniques can be used to probe other state-dependent spectroscopic properties.

8a. Fluorescence

It was found that many microbial rhodopsin proteins and their mutants produce measurable fluorescence. For example, fluorescence of an Arch-based reporter may be excited by light with a wavelength between wavelength of 500 and 650 nm, and emission is peaked at 710 nm. The rate of photobleaching of the reporter decreases at longer excitation wavelengths, so one preferable excitation wavelength is in the red portion of the spectrum, near 633 nm. These wavelengths are further to the red than the excitation and emission wavelengths of any other fluorescent protein, a highly desirable property for in vivo imaging. Preferably, the fluorescence of the reporter shows negligible photobleaching, in stark contrast to all other known fluorophores. When excited at 633 nm, the reporter and GFP emit a comparable numbers of photons prior to photobleaching. Thus microbial rhodopsins constitute a new class of highly photostable, membrane-bound fluorescent markers. It may be found that fluorescence of the reporter is sensitive to the state of protonation of the Schiff base in that the protonated form preferentially fluoresces. Thus voltage-induced changes in protonation enhance changes in fluorescence. In some embodiments, the fluorescence of the reporter is detected using e.g., a fluorescent microscope, a fluorescent plate reader, FACS sorting of fluorescent cells, etc.

8b. Electrochromic Fluorescence Resonance Energy Transfer (eFRET)

FRET is a useful tool to quantify molecular dynamics in biophysics and biochemistry, such as protein-protein interactions, protein-DNA interactions, and protein conformational changes. For monitoring the complex formation between two molecules (e.g., retinal and microbial rhodopsin), one of them is labeled with a donor and the other with an acceptor, and these fluorophore-labeled molecules are mixed. When they are dissociated, the donor emission is detected upon the donor excitation. On the other hand, when the donor and acceptor are in proximity (1-10 nm) due to the interaction of the two molecules, the acceptor emission is predominantly observed because of the intermolecular FRET from the donor to the acceptor.

A fluorescent molecule appended to a microbial rhodopsin can transfer its excitation energy to the retinal, but only if the absorption spectrum of the retinal overlaps with the emission spectrum of the fluorophore. Changes in the absorption spectrum of the retinal lead to changes in the fluorescence brightness of the fluorophore. To perform electrochromic FRET, a fluorescent protein is fused with the microbial rhodopsin voltage sensor, and the fluorescence of the protein is monitored. This approach has the advantage over direct fluorescence that the emission of fluorescent proteins is far brighter than that of retinal, but the disadvantage of being an indirect readout, with smaller fractional changes in fluorescence.

In some embodiments, voltage-induced changes in the absorption spectrum of microbial rhodopsins are detected using electrochromic FRET.

8c. Rhodopsin Optical Lock-In Imaging (ROLI)

The absorption spectrum of many of the states of retinal is temporarily changed by a brief pulse of light. In ROLI, periodic pulses of a “pump” beam are delivered to the sample. A second “probe” beam measures the absorbance of the sample at a wavelength at which the pump beam induces a large change in absorbance. Thus the pump beam imprints a periodic modulation on the transmitted intensity of the probe beam. These periodic intensity changes are detected by a lock-in imaging system. In contrast to conventional absorption imaging, ROLI provides retinal-specific contrast. Modulation of the pump at a high frequency allows detection of very small changes in absorbance.

In some embodiments, the voltage-induced changes in the absorption spectrum of a microbial rhodopsin are detected using rhodopsin optical lock-in imaging.

8d. Raman

Raman spectroscopy is a technique that can detect vibrational, rotational, and other low-frequency modes in a system. The technique relies on inelastic scattering of monochromatic light (e.g., a visible laser, a near infrared laser or a near ultraviolet laser). The monochromatic light interacts with molecular vibrations, phonons or other excitations in the system, resulting in an energy shift of the laser photons. The shift in energy provides information about the phonon modes in the system.

Retinal in microbial rhodopsin molecules is known to have a strong resonant Raman signal. This signal is dependent on the electrostatic environment around the chromophore, and therefore is sensitive to voltage.

In some embodiments, voltage-induced changes in the Raman spectrum of microbial rhodopsins are detected using Raman microscopy.

8e. Second Harmonic Generation (SHG)

Second harmonic generation, also known in the art as “frequency doubling” is a nonlinear optical process, in which photons interacting with a nonlinear material are effectively “combined” to form new photons with twice the energy, and therefore twice the frequency and half the wavelength of the initial photons.

SHG signals have been observed from oriented films of bacteriorhodopsin in cell membranes. SHG is an effective probe of the electrostatic environment around the retinal in optical voltage sensors. Furthermore, SHG imaging involves excitation with infrared light which penetrates deep into tissue. Thus SHG imaging can be used for three-dimensional optical voltage sensing using the optical reporters described herein.

In some embodiments, voltage-induced changes in the second harmonic spectrum of microbial rhodopsins are detected using SHG imaging.

8f. Photothermal Imaging

Photothermal imaging senses the change in refractive index in a medium arising from a change in temperature, where the change in temperature is induced by optical absorption. In photothermal imaging, a “pump” beam of light is absorbed by a sample and generates local heating. A second “probe” beam of light, at a wavelength that is not absorbed by the sample, propagates through the sample. Temperature-induced changes in the optical path length are detected by one of several optical configurations, e.g. Schlieren imaging or differential interference contrast (DIC) microscopy.

In some embodiments, photothermal imaging is used to detect voltage-induced changes in the absorption spectrum of a microbial rhodopsin.

8g. Chromophore

In the wild, microbial rhodopsins contain a bound molecule of retinal which serves as the optically active element. These proteins will also bind and fold around many other chromophores with similar structure, and possibly preferable optical properties. Analogues of retinal with locked rings cannot undergo trans-cis isomerization, and therefore have higher fluorescence quantum yields (Brack et al., Picosecond time-resolved adsorption and fluorescence dynamics in the artificial bacteriorhodopsin pigment BR6.11, Biophys. J. 65(2):964-972). Analogues of retinal with electron-withdrawing substituents have a Schiff base with a lower pKa than natural retinal and therefore may be more sensitive to voltage (Sheves et al., 1986, Controlling the pKa of the bacteriorhodopsin Schiff base by use of artificial retinal analogs, PNAS 83(10):3262-3266; Rousso et al., 1995, pKa of the protonated Schiff base and asparatic 85 in the Bacteriorhodopsin binding site is controlled by a specific geometry between the two resdidues, Biochemistry 34(37):12059-12065). Covalent modifications to the retinal molecule may lead to optical voltage sensors with significantly improved optical properties and sensitivity to voltage.

### 9. Systems of the Invention

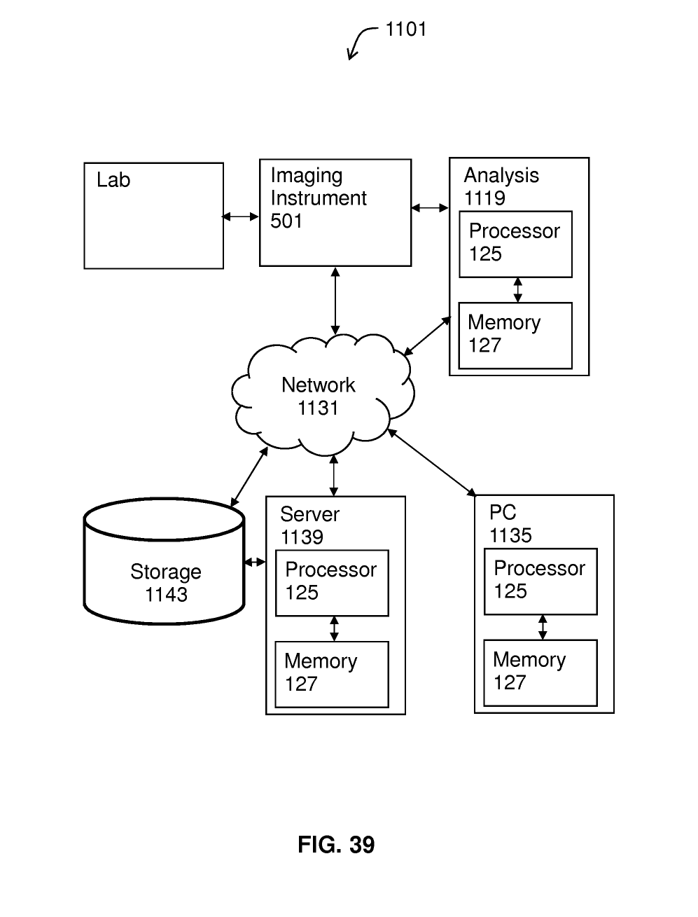

FIG. 39 presents a system 1101 useful for performing methods of the invention. Results from a lab (e.g., transformed, converted patient cells) are loaded into imaging instrument 501. Imaging instrument 501 is operably coupled to an analysis system 1119, which may be a PC computer or other device that includes a processor 125 coupled to a memory 127. A user may access system 1101 via PC 1135, which also includes a processor 125 coupled to a memory 127. Analytical methods described herein may be performed by any one or more processor 125 such as may be in analysis system 1119, PC 1135, or server 1139, which may be provided as part of system 1101. Server 1139 includes a processor 125 coupled to a memory 127 and may also include optional storage system 1143. Any of the computing device of system 1101 may be communicably coupled to one another via network 1131. Any, each, or all of analysis system 1119, PC 1135, and server 1139 will generally be a computer. A computer will generally include a processor 125 coupled to a memory 127 and at least one input/output device.

A processor 125 will generally be a silicon chip microprocessor such as one of the ones sold by Intel or AMD. Memory 127 may refer to any tangible, non-transitory memory or computer readable medium capable of storing data or instructions, which—when executed by a processer 125—cause components of system 1101 to perform methods described herein. Typical input/output devices may include one or more of a monitor, keyboard, mouse, pointing device, network card, Wi-Fi card, cellular modem, modem, disk drive, USB port, others, and combinations thereof. Generally, network 1131 will include hardware such as switches, routers, hubs, cell towers, satellites, landlines, and other hardware such as makes up the Internet.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Imaging in Cardiomyocytes

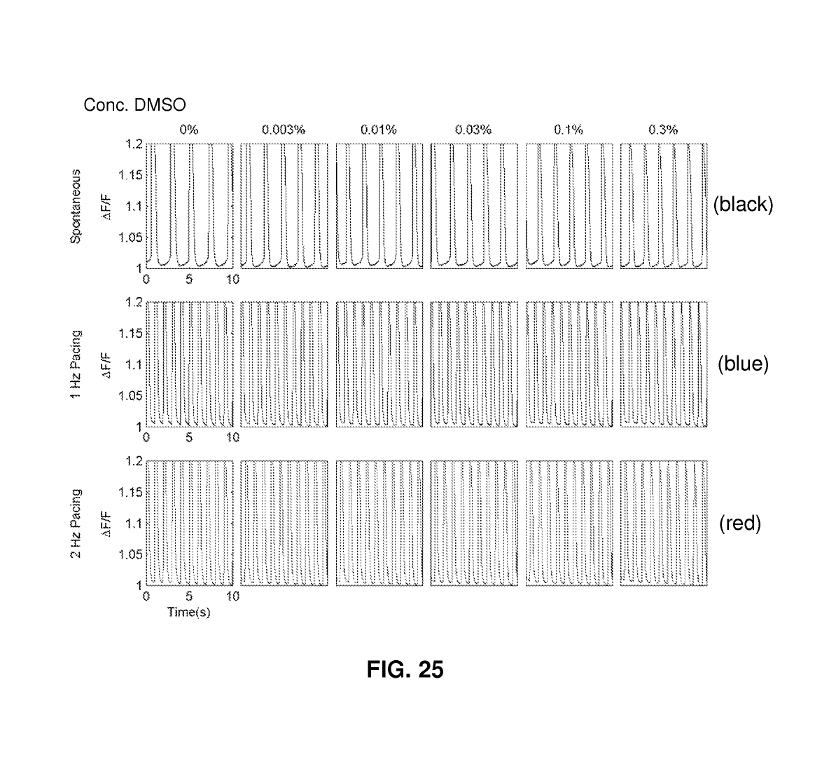

Methods and systems of the invention may be used to characterize cardiac cells. A cell can be obtained and converted into a cardiomyocyte. For example, using methods described herein, fibroblasts may be converted to cardiomyocytes via induced pluripotent stem cells. An optical actuator of electrical activity, an optical reporter of electrical activity, or both may be incorporated into any one or more of cardiomyocytes as described above. As shown in FIGS. 25-30, a signal may be obtained from the optical reporter in response to a stimulation of the cardiomyocytes. By evaluating the signal, the cardiomyocytes are characterized.

FIG. 25 demonstrates effects of DMSO vehicle control on the action potential (AP) waveforms of hiPSC-derived cardiomyocytes. Representative segments of the mean fluorescence (AF/F) versus time (seconds, s) traces at each concentration (0% ‘blank’, 0.003%, 0.01%, 0.03%, 0.1% and 0.3% DMSO) are shown for spontaneously beating cells (top panel) as well as the same cells optogenetically paced at 1 Hz (middle panel) and 2 Hz (bottom panel). Traces are taken from a single dish of cells and a single field-of-view. Data was taken at 100 Hz frame rate.

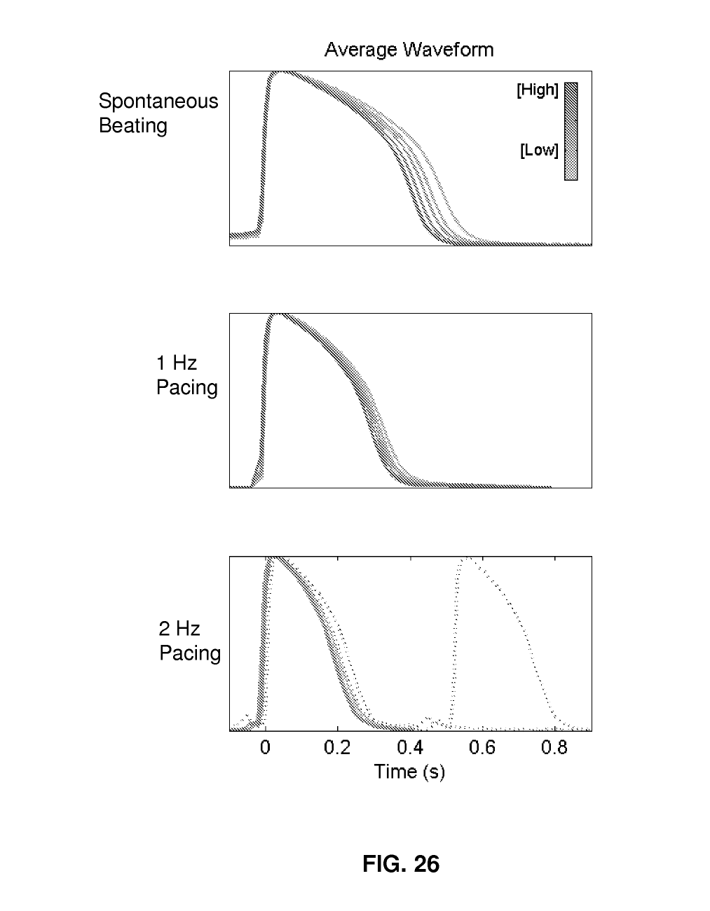

FIG. 26 presents the effects of DMSO control vehicle on the average AP waveform. The average waveform for the range of concentrations tested (cyan to magenta; lowest to highest concentrations tested, respectively) is shown. The top, middle, and bottom panels correspond to spontaneous beating, 1 Hz pacing and 2 Hz pacing, respectively. Dashed lines indicate that the cells did not beat at the specified pacing rate. In the case of spontaneous beating, this criterion did not apply. Panels are calculated from data taken at 100 Hz.

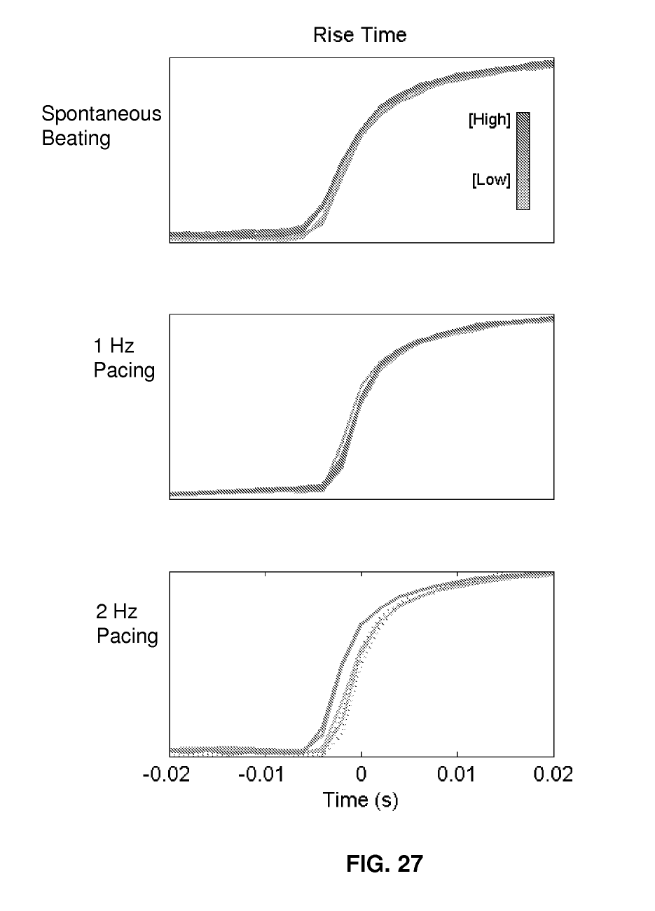

FIG. 27 presents the effects of DMSO control vehicle on the average rise time. The average rise time for the range of concentrations tested (cyan to magenta; lowest to highest concentrations tested, respectively) is shown. The top, middle, and bottom panels correspond to spontaneous beating, 1 Hz pacing and 2 Hz pacing, respectively. Dashed lines indicate that the cells did not beat at the specified pacing rate. In the case of spontaneous beating, this criterion did not apply. Panels are calculated from data taken at 500 Hz.

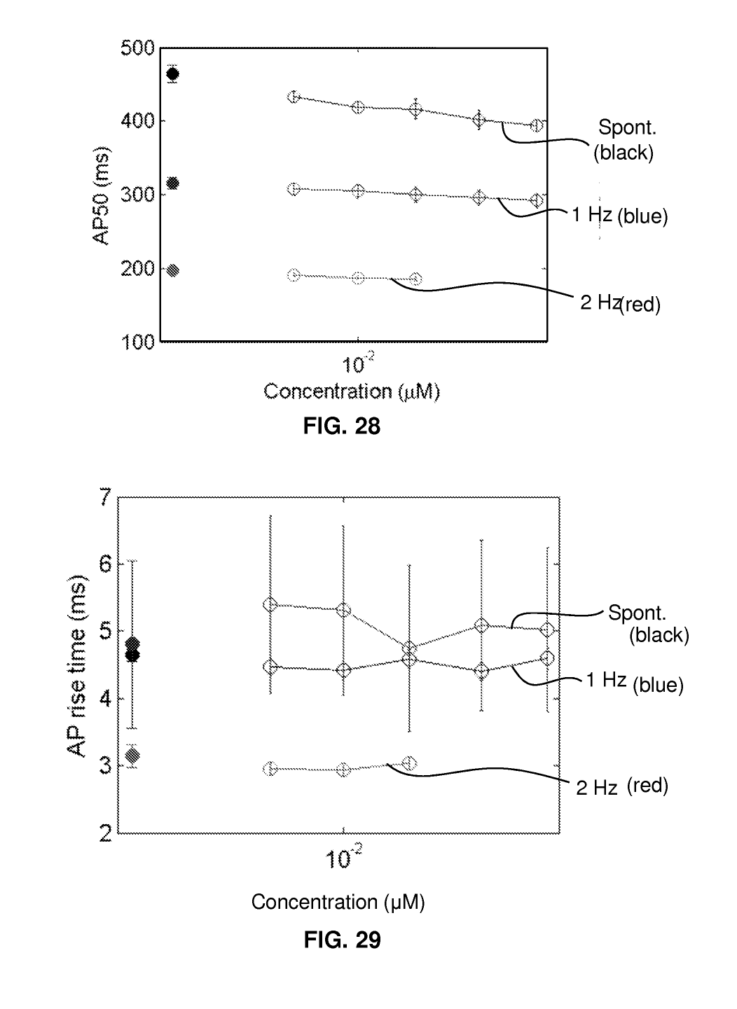

FIGS. 28-31 illustrate the quantification of the effect of DMSO addition on AP waveform.

FIG. 28 shows the dose dependence of the action potential duration at 50% of repolarization (AP50).

FIG. 29 shows the dose dependence of the action potential duration at 90% of repolarization (AP90).

FIG. 30 shows the dose dependence of the AP rise time.

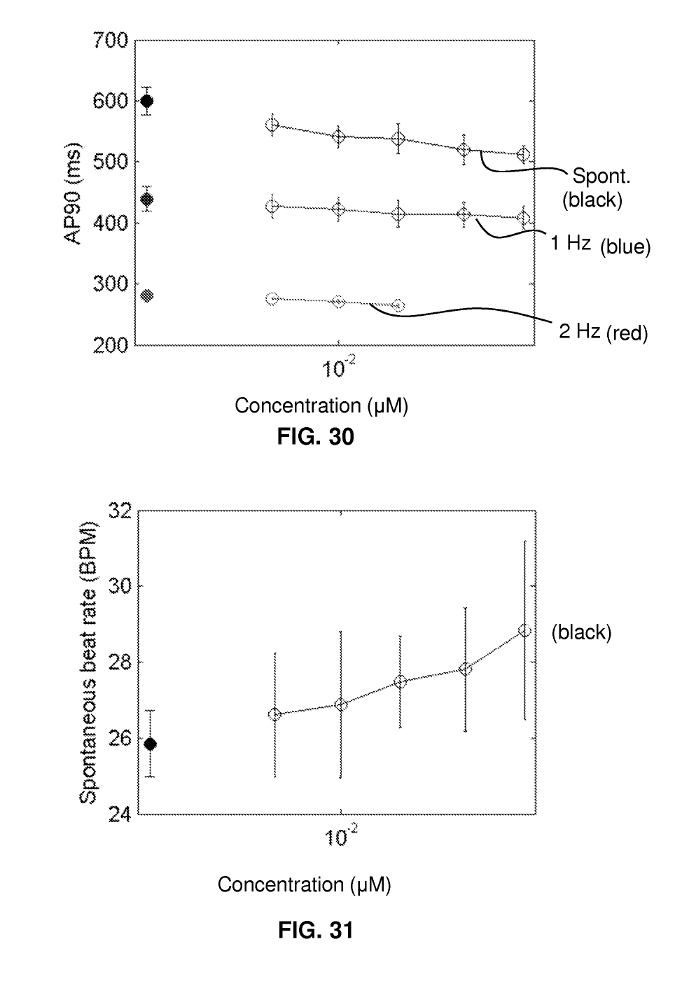

FIG. 31 shows the dose dependence of the spontaneous beat rate.

In FIGS. 28-31, closed circles are used to represent the ‘blank’ addition of imaging buffer alone whereas open circles are used to represent the addition of compound at varying concentrations. Analysis was performed on fluorescence versus time traces acquired under conditions of spontaneous beating (black) as well as pacing regimens of 1 Hz (blue) and 2 Hz (red). Note that in the case of 1 Hz and 2 Hz pacing, data points are omitted from the plot in the event that the cells do not pace at the specified pace rate. Data points are also omitted in the event that the cells stop beating. Data and error bars are reported as the mean+/−standard error of the mean.

### Example 2

### Imaging in HEK Cells

An optical reporter such as Arch 3 may be expressed in human embryonic kidney 293 (HEK293T) cells. Fluorescence of Arch 3 in HEK 293T cells was readily imaged in an inverted fluorescence microscope with red illumination (=640 nm, I=540 W/cm2), a high numerical aperture objective, a Cy5 filter set, and an EMCCD camera.

FIG. 32 shows a model of Arch as a voltage sensor. pH and membrane potential can both alter the protonation of the Schiff base. The crystal structure shown is bacteriorhodopsin; the structure of Arch has not been solved.

FIG. 33 shows absorption (solid line) and fluorescence emission (Em, see, dashed line) spectra of purified Arch at neutral and high pH.

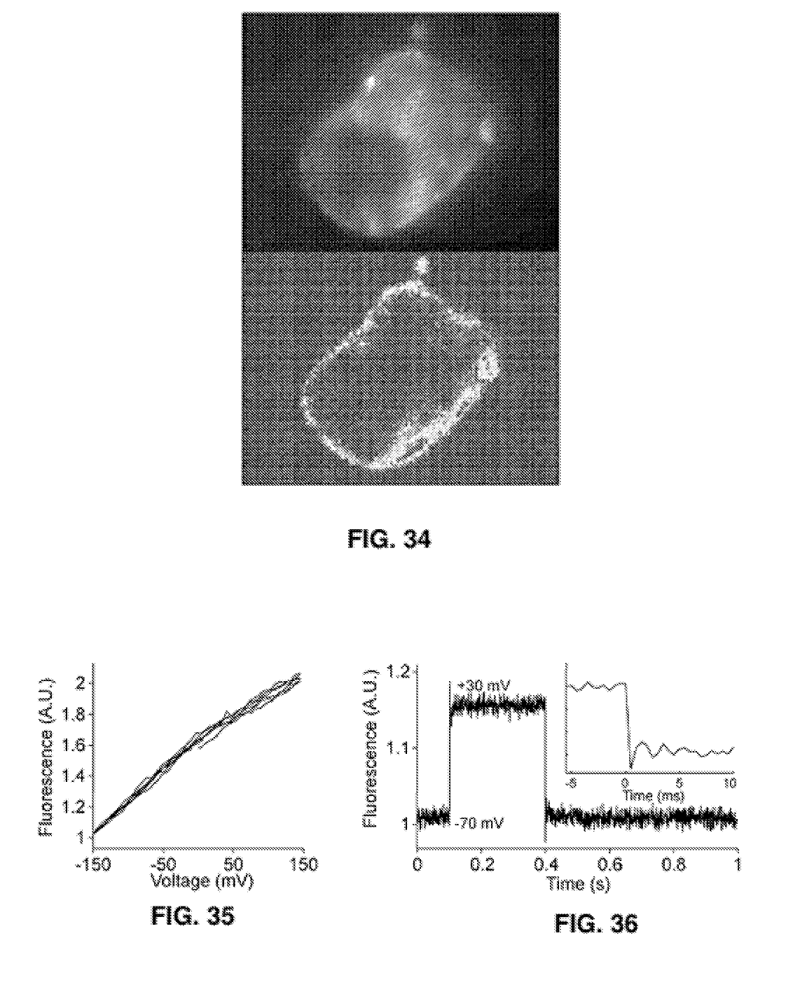

FIG. 34 top shows a HEK cell expressing Arch, visualized via Arch fluorescence. FIG. 34 bottom shows a pixel-weight matrix regions of voltage-dependent fluorescence. Scale bar 10 μm.

Fluorescence of Arch 3 in HEK 293 cells was readily imaged in an inverted fluorescence microscope with red illumination (lambda=640 nm, I=540 W/cm̂2), a high numerical aperture objective, a Cy5 filter set, and an EMCCD camera. The cells exhibited fluorescence predominantly localized to the plasma membrane (FIG. 34). Cells not expressing Arch were not fluorescent. Cells showed 17% photobleaching over a continuous 10-minute exposure, and retained normal morphology during this interval.

The fluorescence of HEK cells expressing Arch was highly sensitive to membrane potential, as determined via whole-cell voltage clamp. We developed an algorithm to combine pixel intensities in a weighted sum such that the output, was a nearly optimal estimate of membrane potential V determined by conventional electrophysiology. FIG. 34 shows an example of a pixel-weight matrix, indicating that the voltage-sensitive protein was localized to the cell membrane; intracellular Arch contributed fluorescence but no voltage-dependent signal.

FIG. 35 shows fluorescence of Arch as a function of membrane potential. The fluorescence was divided by its value at −150 mV. The fluorescence increased by a factor of 2 between −150 mV and +150 mV, with a nearly linear response throughout this range (FIG. 35). The response of fluorescence to a step in membrane potential occurred within the 500 micro s time resolution of our imaging system on both the rising and falling edge.

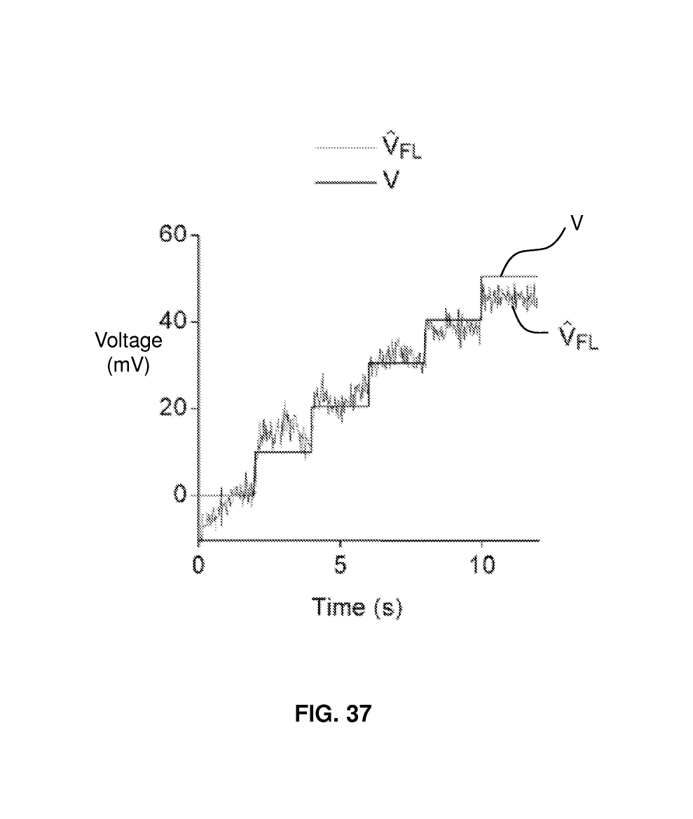

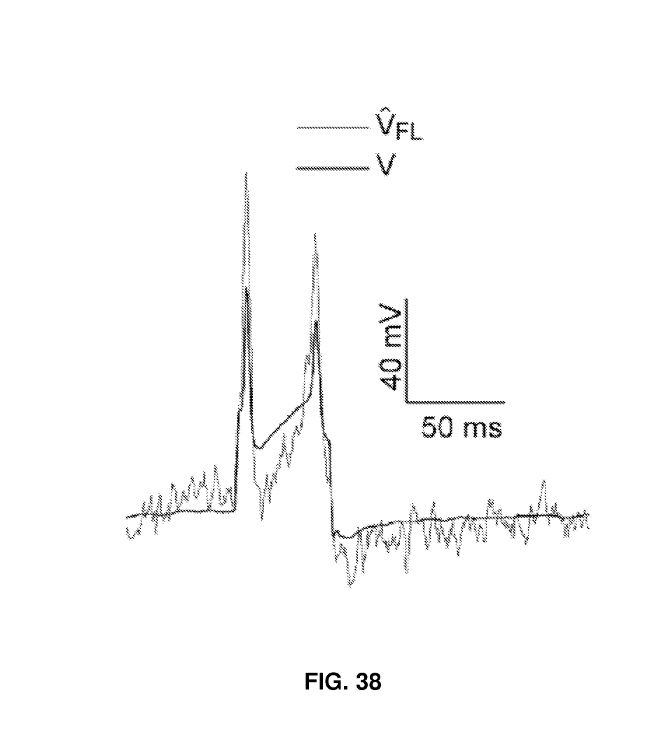

FIG. 36 shows dynamic response of Arch to steps in membrane potential between −70 mV and +30 mV. The overshoots on the rising and falling edges were an artifact of electronic compensation circuitry. Data were an average of 20 cycles. Inset shows that step response occurred in less than the 0.5 ms resolution of the imaging system. The cells exhibited fluorescence predominantly localized to the plasma membrane (FIG. 34). Cells not expressing Arch 3 were not fluorescent. Cells showed 17% photobleaching over a continuous 10-minute exposure, and retained normal morphology during this interval. Application of a sinusoidally varying membrane potential led to sinusoidally varying fluorescence; at f=1 kHz, the fluorescence oscillations retained 55% of their low-frequency amplitude (FIG. 37). Arch reported voltage steps as small as 10 mV, with an accuracy of 625 micro V/(Hz)̂(1/2) over timescales<12 s (FIG. 38). Over longer timescales laser power fluctuations and cell motion degraded the accuracy.

FIG. 37 shows sensitivity of Arch 3 WT to voltage steps of 10 mV. Whole-cell membrane potential determined via direct voltage recording, V, (bolded black line, showing step-like line on the graph) and weighted Arch 3 fluorescence, V′FL, (solid narrower line showing serrations on the graph).

FIG. 38 shows that Arch 3 reports action potentials without exogenous retinal. We made an image of 14 day in vitro (DIV) hippocampal neuron imaged via Arch 3 fluorescence with no exogenous retinal. Electrical (bolded solid black line) and fluorescence (non-bolded line, showing serrated line in the graph) records of membrane potential from the neuron during a current pulse. Action potentials are clearly resolved.

### Example 3

### CaViar

1. Setup for Optical Pacing with Voltage and Calcium Reporting

Methods of the invention may be used to characterize the effects of a compound on cells by obtaining a sample that includes cells that include an optical voltage reporter and cells that include a light-gated ion channel, exposing the sample to a compound, and illuminating the cells that include the light-gated ion channel. An optical signal is detected from the cells that include the optical voltage reporter and an effect of the compound on the cells is characterized by comparing the optical signal to a reference.

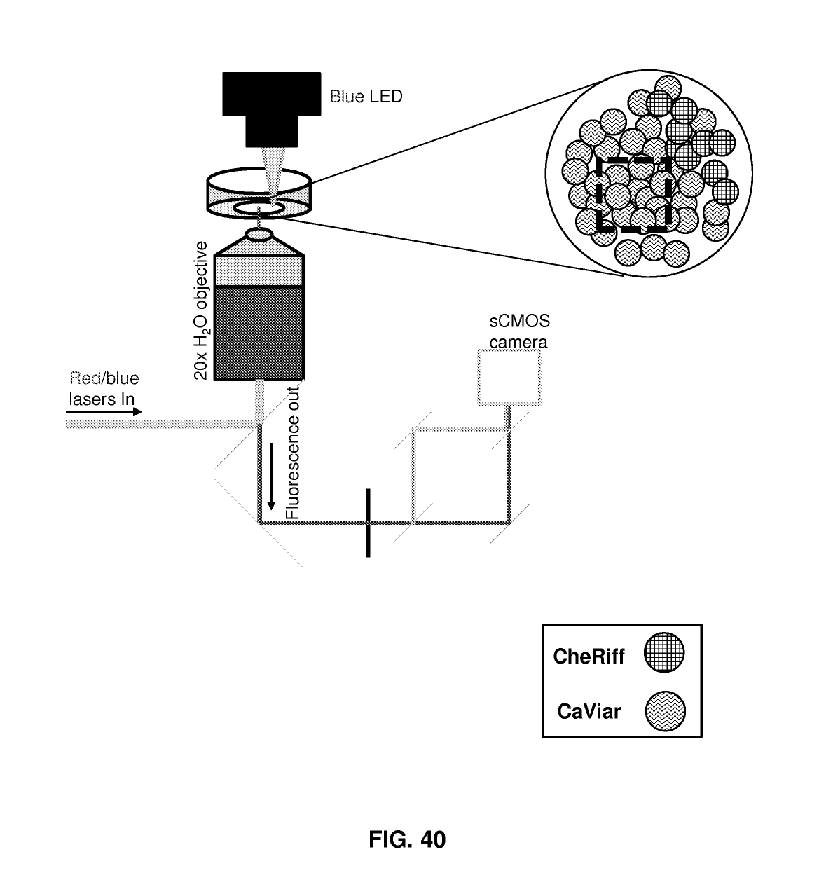

FIG. 40 diagrams a microscopy setup for illuminating the cellular sample 4001. Cells expressing a light-gated ion channel such as CheRiff are plated proximal to reporter cells that have the voltage reporter (which may be, for example, QuasAr). Thus the setup will detect and evaluate signal propagation between the pre-synaptic cells and the reporter cells. In some embodiments, the reporter cells also include an optical ion sensor that indicates an intracellular concentration of an ion, for example, a calcium reporter. In a preferred embodiment, the optical voltage reporter and the calcium reporter are provided together within a fusion protein. For example, the fusion protein may be CaViar which includes a calcium reporter and a voltage indicator. Using such a fusion protein, detecting the optical signal may include detecting an action potential and calcium transients. One exemplary set of proteins for the depicted assay uses an algal channelrhodopsin for the light-gated ion channel, a microbial rhodopsin for the optical voltage reporter, and a GCaMP variant for the calcium reporter (e.g., preferably with the microbial rhodopsin and the GCaMP variant fused in a single protein). Using a fusion protein may be to ensure that calcium and voltage measurements scale with one another and are thus comparable across samples.

Optionally, illuminating the cells may be done using spatially resolved light from a digital micromirror device (DMD). The illuminating the cells and detecting the signal may be simultaneous. The measurements from the depicted assay may be analyzed by a computer system 1101. The computer system 1101 can detect individual action potentials (e.g., by performing an independent component analysis and identifying a spike train associated with the cell).

### 2. Cardiomyocytes and Pentamidine

The assay may be used for cardiac tissue/cardiotoxicity screening. For example, the cells that include the optical voltage reporter may include cardiomyocytes.

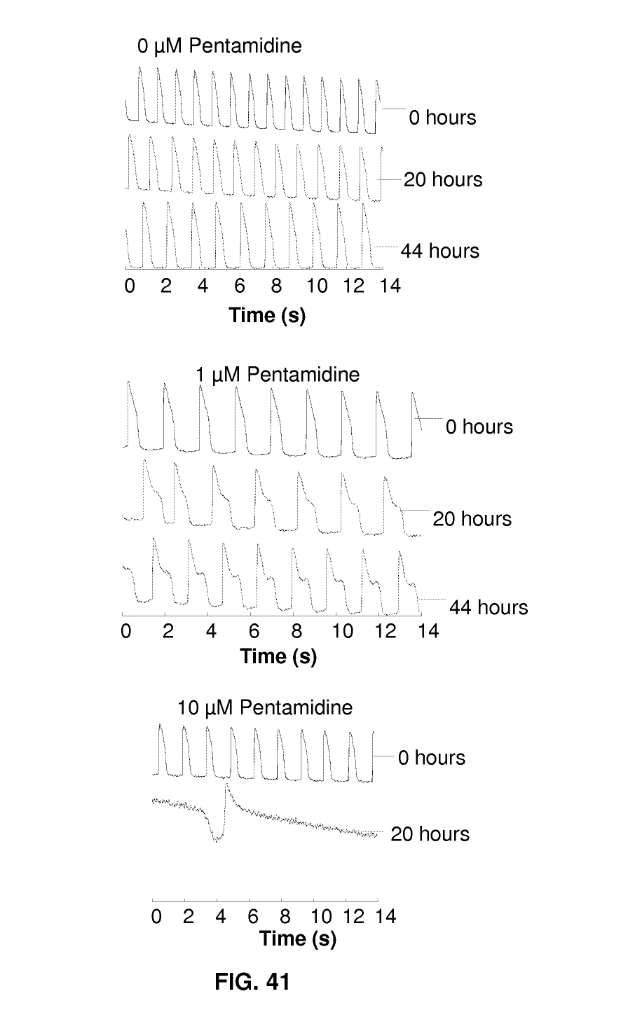

FIG. 41 illustrates the action potentials obtained by exposing cardiomyocytes to a compound that blocks hERG trafficking, pentamidine. The data were obtained by exposing the cellular samples to 1 μM pentamidine (in DMSO), 10 μM pentamidine, and a DMSO control. Readings were taken at 0 hours, 20 hours, and 44 hours after exposure. The control in the top panel of FIG. 41 shows a baseline result from untreated cells. Upon exposure to 1 μM pentamidine, after 20 hours, early after depolarizations (EADs) are evident. Also, AP90 prolongation is revealed. I.e., pentamidine is shown to reduce hERG trafficking After exposure to 10 μM, beating stops. Preferably, the computer system 1101 is used to characterize the effect of the compound (e.g., pentamidine) on the cells by comparing the action potential and the calcium transients to those of a control and measuring a difference (e.g., for early after depolarization (EAD), action potential rise time, QT prolongation, alternans; cessation of beating, AP50, AP90, beat rate, maximal upstroke velocity, or combinations thereof). Detecting the signal may include capturing an image of the sample and using a computer system to detect individual action potentials for each of a plurality of the cells that include the optical voltage reporter.

### 3. CiPA Data

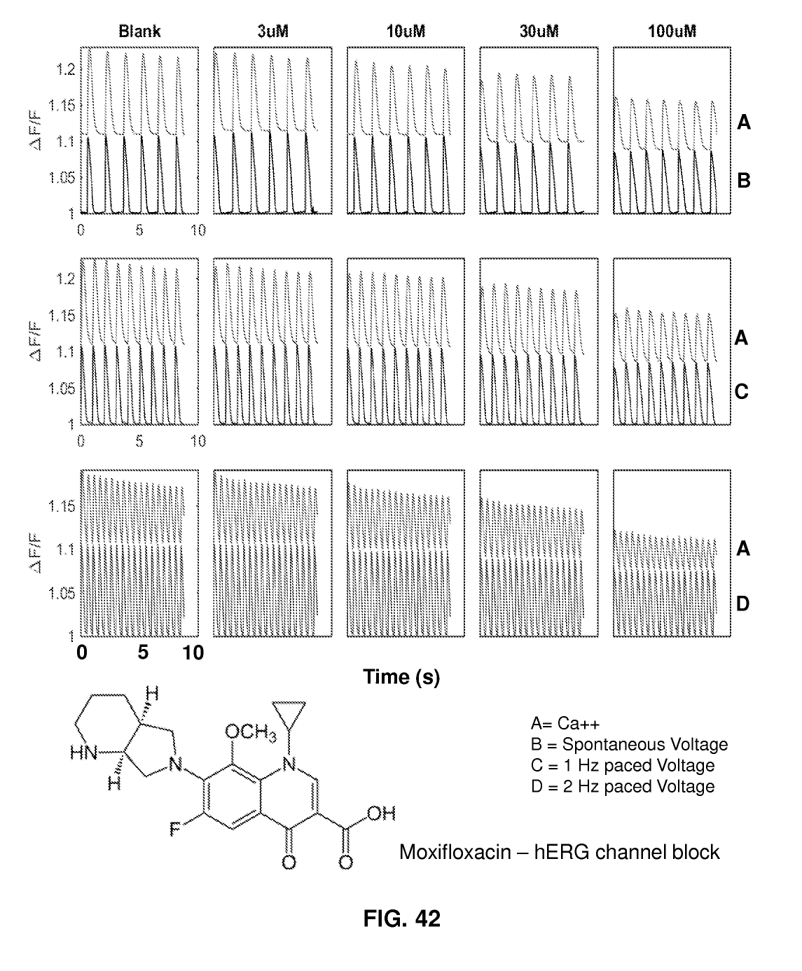

FIGS. 42-47 relate to an optical Comprehensive in vitro Proarrythmia Assay (CiPA).

FIG. 42 shows results from exposing cardiomyocytes to a hERG channel block (e.g., moxifloxacin). The columns show results from increasing concentrations and each panel is fluorescence over time. The top row shows a calcium trace with spontaneously beating cells. The middle row shows calcium and voltage for cells paced at 1 Hz (e.g., by being plated as shown in FIG. 40 and stimulating the cells that include the light-gated ion channel (e.g., CheRiff) with blue light at 1 Hz. The bottom row includes the results from pacing at 2 Hz. It can be seen that at high concentrations of moxifloacin, the calcium is severely diminished with a less pronounced effect on voltage. Thus the use of CaViar is greater than the sum of its parts, as the voltage trace reveals action potentials while the calcium shows channel blockage.

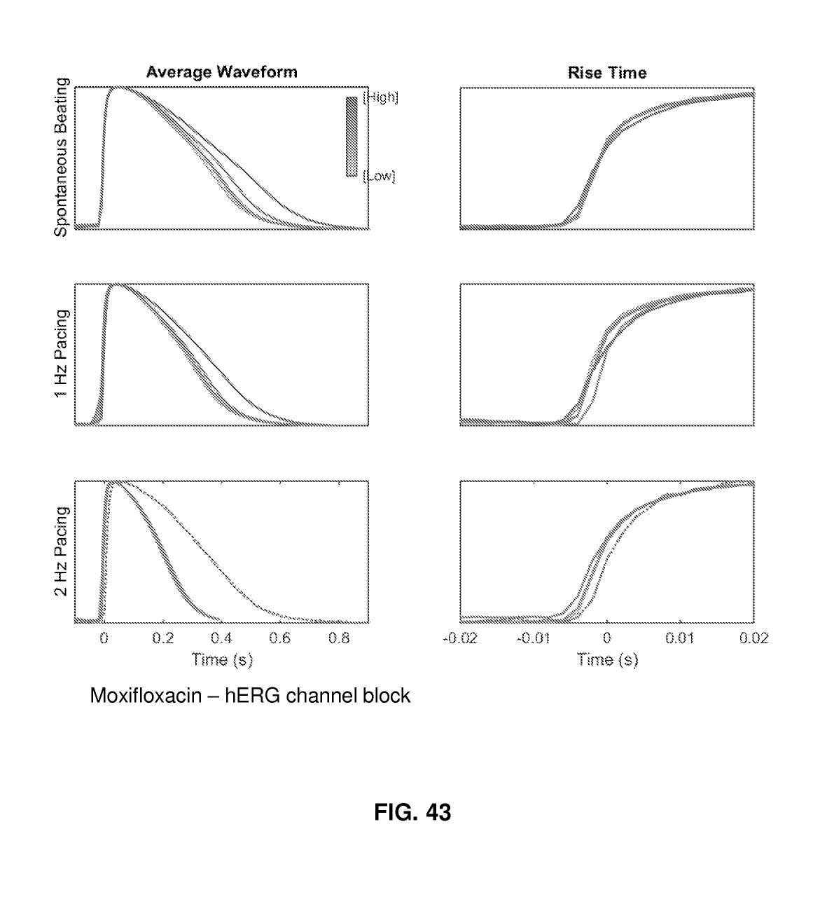

FIG. 43 gives the average waveform and rise times for those results. It can be seen that under 2 Hz pacing, the average waveforms diverge dramatically, revealing an AP prolongation.

FIG. 44 gives summary statistics for freely beating cardiomyocytes and paced cardiomyocytes upon exposure to different concentrations of moxifoxacin. Those results show that exposure results in a decrease in AP30, decrease in AP60, decrease in AP90, increase in rise time, and decrease in Ca amplitude.

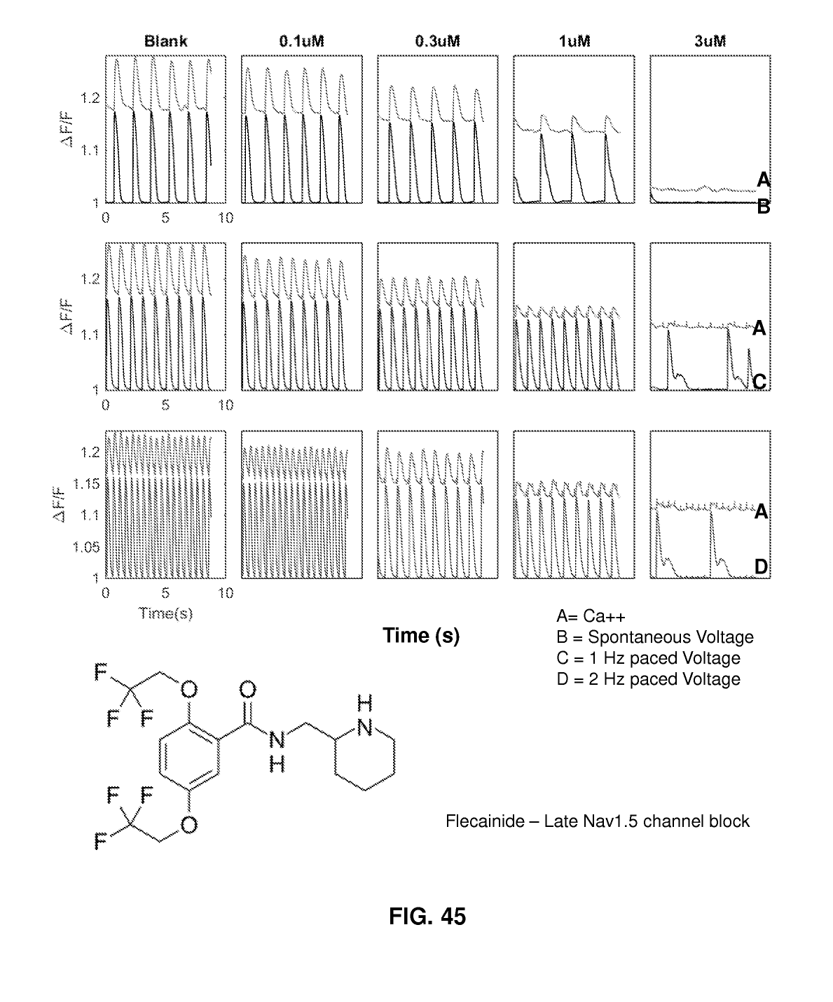

FIG. 45 shows results from exposing cardiomyocytes to a late Nav1.5 channel block (e.g., flecainide). The columns show results from increasing concentrations and each panel is fluorescence over time. The top row shows a calcium trace with spontaneously beating cells. The middle row shows calcium and voltage for cells paced at 1 Hz (e.g., by being plated as shown in FIG. 40 and stimulating the cells that include the light-gated ion channel (e.g., CheRiff) with blue light at 1 Hz. The bottom row includes the results from pacing at 2 Hz. It can be seen that at high concentrations of flecainide, calcium and voltage are dramatically diminished.

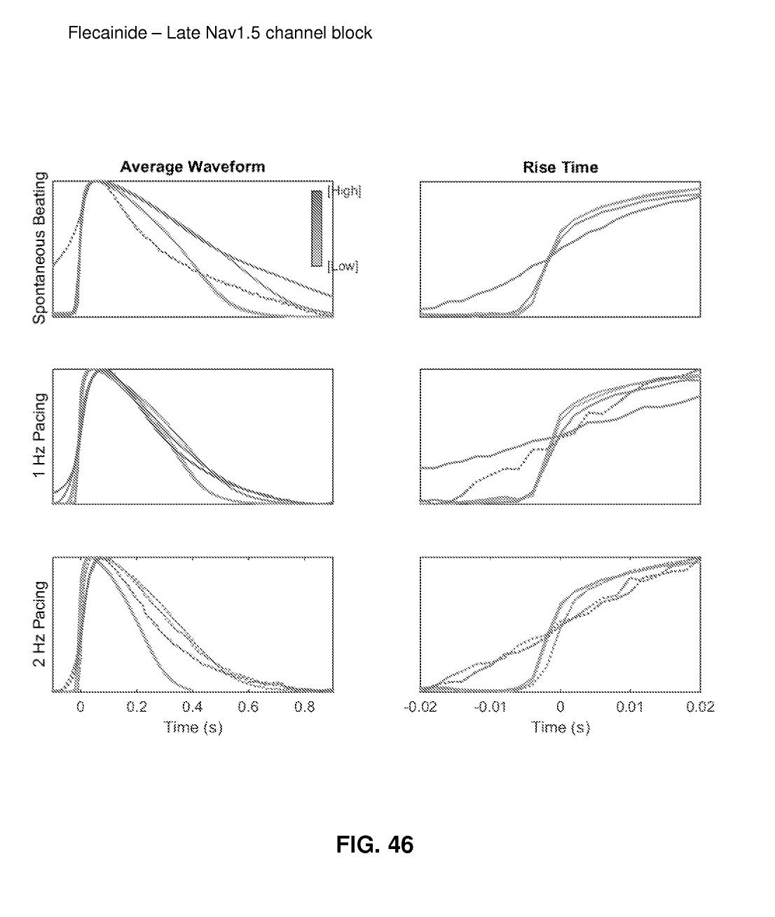

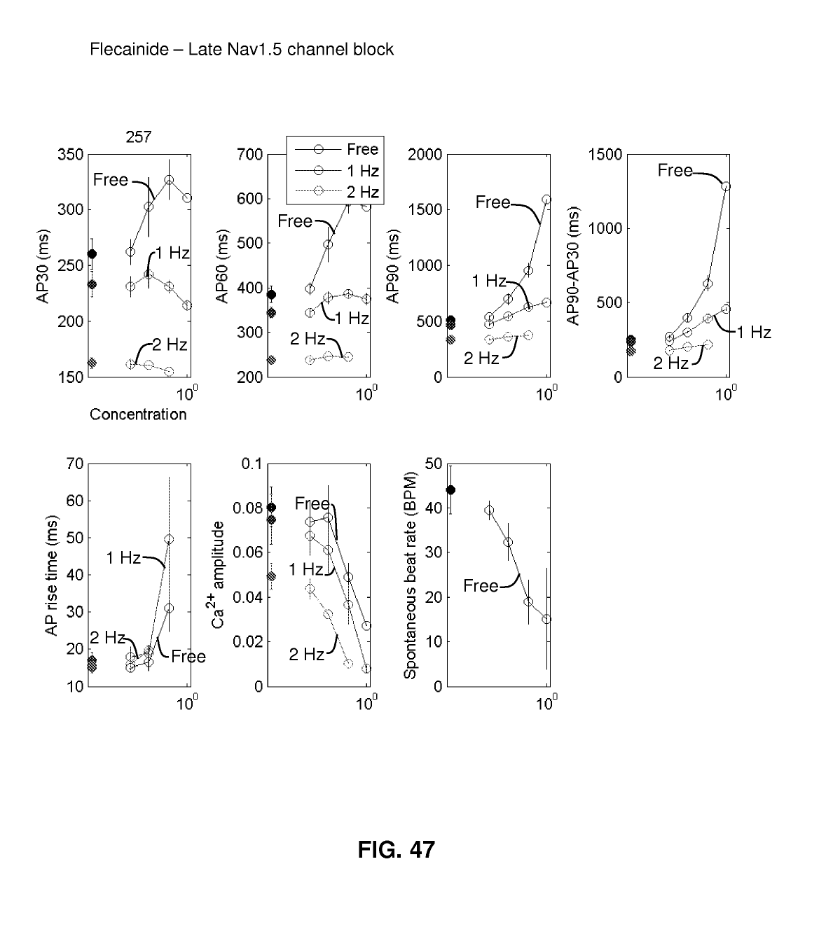

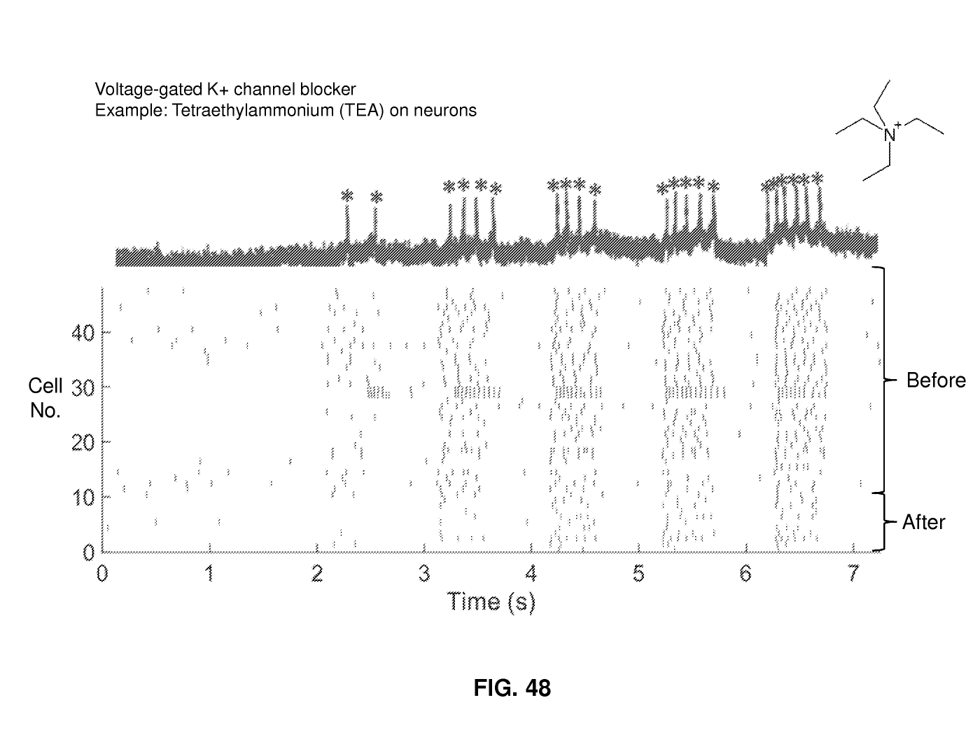

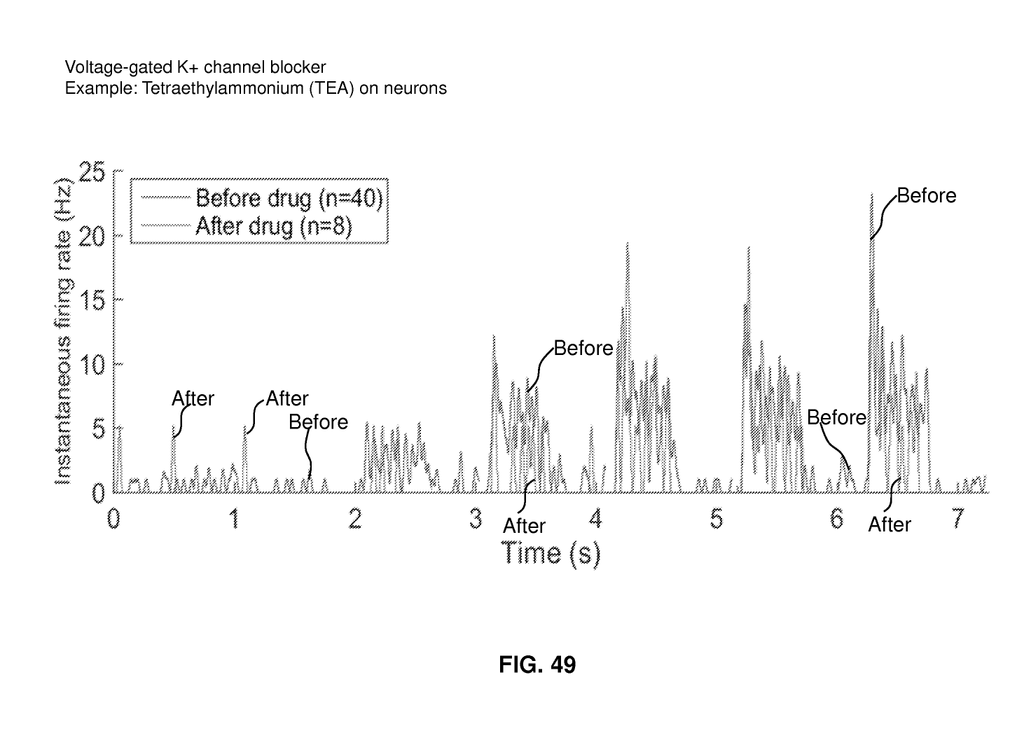

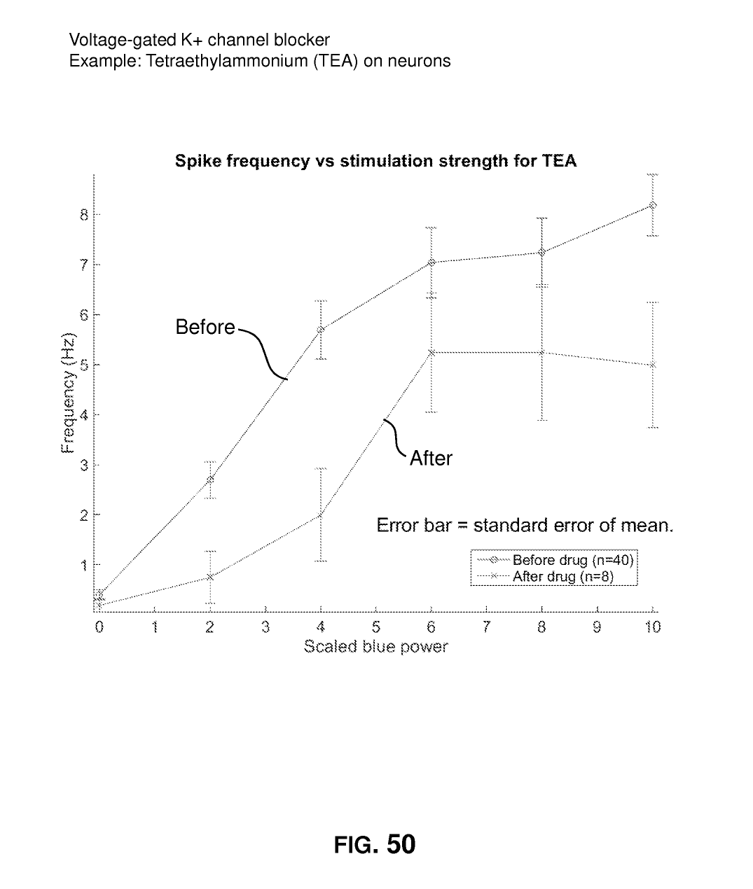

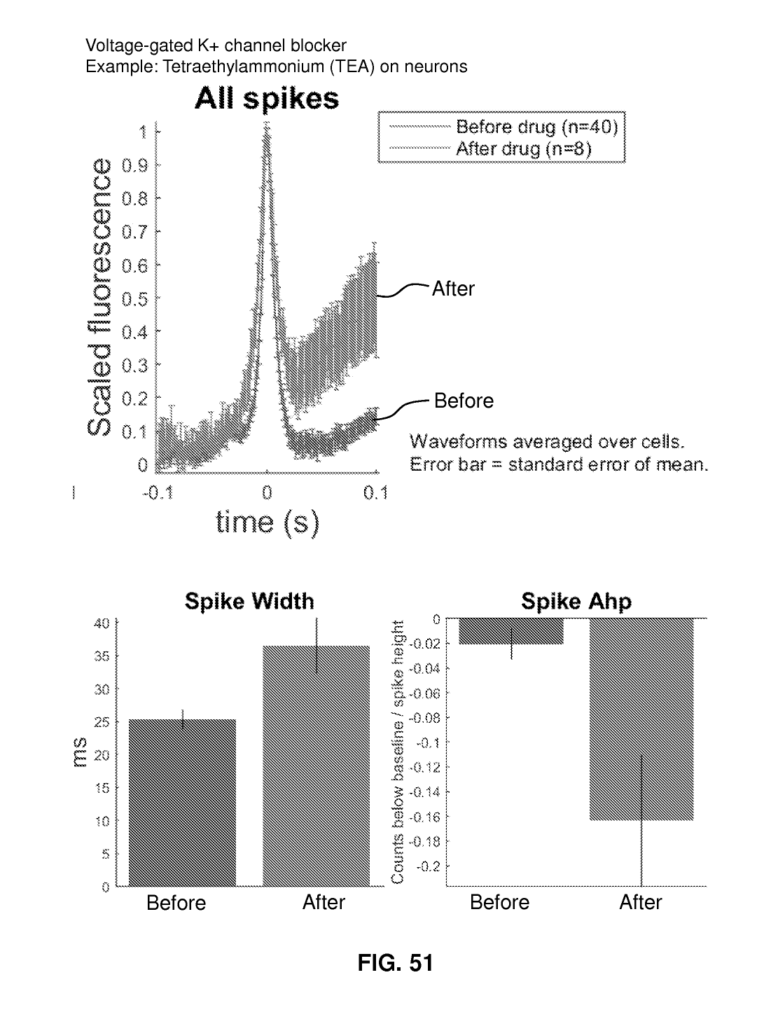

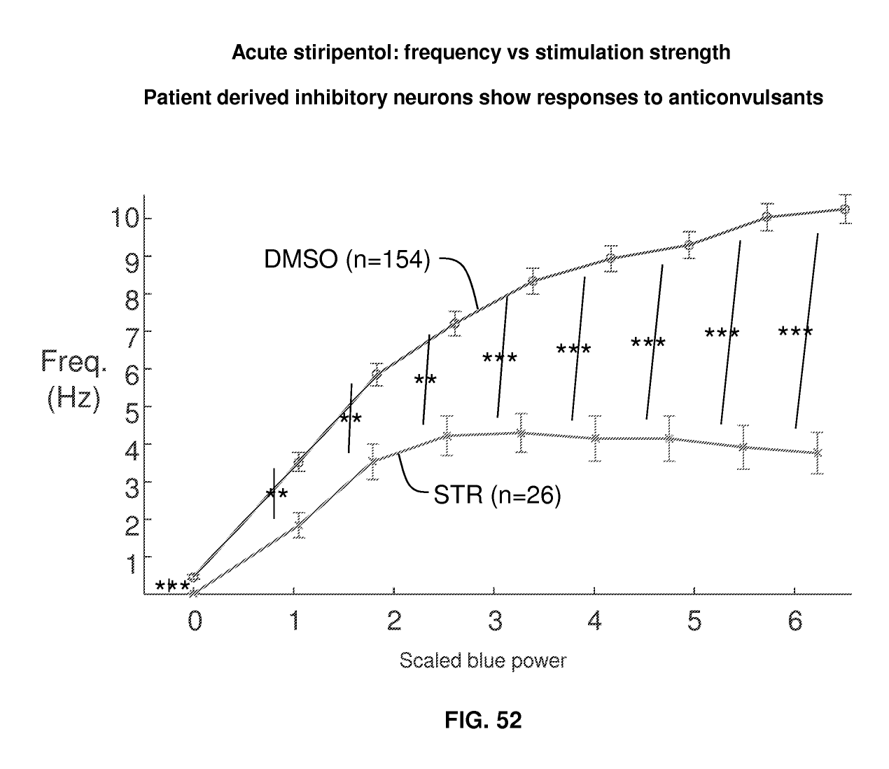

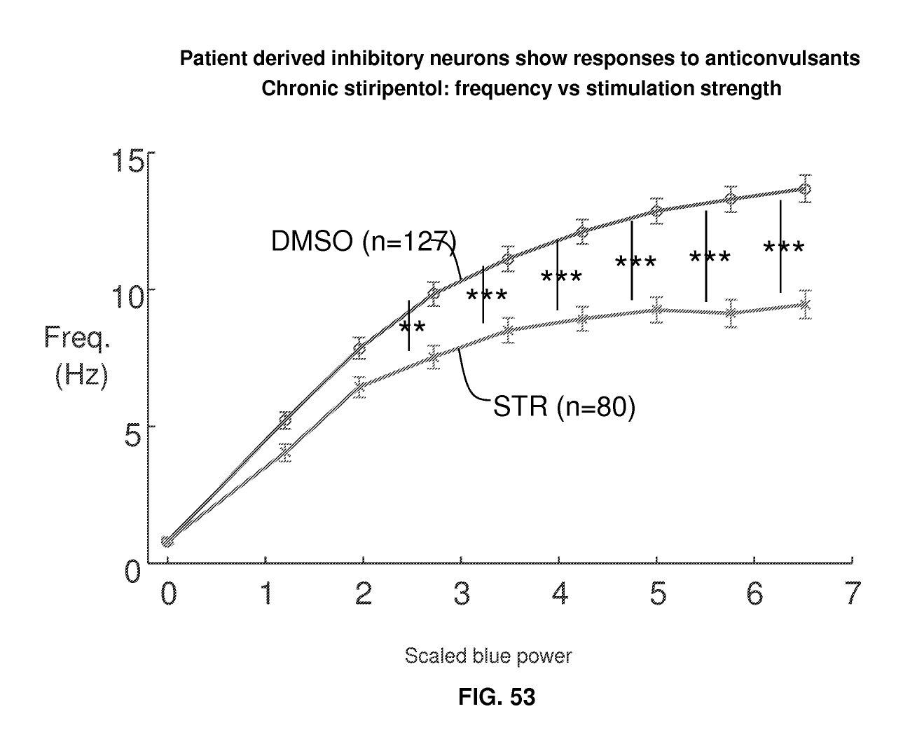

FIG. 46 gives the average waveform and rise times for those results. It can be seen that under 2 Hz pacing, the average waveforms diverge dramatically and that under any input conditions (spontaneous beating or paced) that flecainide interferes with rise time.

FIG. 44 gives summary statistics for freely beating cardiomyocytes and paced cardiomyocytes upon exposure to different concentrations of flecainide. Those results show that exposure results in a decrease in AP30, decrease in AP60, decrease in AP90, increase in rise time, and decrease in Ca amplitude. Thus it can be seen that compositions and methods of the invention can be used to screen for the effects of compounds on cardiomyocytes and that the results provide many qualities of data.

### 4. Neurons

Methods of the invention may be used to evaluate the effects of compounds on neurons. In some embodiments, the cells that include the optical voltage reporter are neurons and those neurons are exposed to a voltage-gated potassium channel blocker such as tetraethylammonium.

FIG. 48 shows the action potential timing for a large number of neurons subject to increasing stimulus intensity over a 7 second period. Each second, a progressively greater number of cells fire. The computer system 1101 detects a cumulative result (trace across top) and identifies spikes (marked with an asterisk).

FIG. 49 plots the instantaneous firing rate in Hz for those cells before and after exposure to tetraethylammonium. At lower intensities (time=0 to about 2 s) the unexposed cells fire at a lower rate, while the exposed cells may be characterized by a few very high spikes in firing rate. After a few seconds, the unexposed cells (“before”) consistently fire at a higher rate.

FIG. 50 graphs spike frequency over unitless scaled stimulus power (i.e., CheRiff is being stimulated by blue light and the input power is normalized and scaled to 10 without any units). On FIG. 50, a clear pattern emerges, exposure to tetraethylammonium clearly decreases spike frequency at all stimulus powers.

FIG. 51 presents the results of measuring various shape parameters of the waveforms for the neurons before and after exposure to tetraethylammonium. It can be seen from the top panel (average waveform) that, after exposure, the fluorescence does not return to baseline to the degree it did before exposure. Additionally, after exposure, the spike width is greater and the height is lower. Thus it is revealed that exposure of neurons to tetraethylammonium leads to a resulting reduction in spike frequency, broadening of AP width, change in Ahp, and a reduction in the number of spiking cells.

### 5. Anticonvulsants

FIG. 52 illustrates the results of exposing patient-derived neurons to acute doses of an anticonvulsant. A frequency of spiking is plotted over unitless scaled stimulus strength. Angled vertical lines are drawn onto the graph to aid in showing which data points correspond to one another. Asterisks are used to indicate a significance of a difference. It can be seen that after exposure to the anticonvulsant stiripentol (STR) in DMSO (versus exposure to the DMSO alone), spike frequency is significantly decreased.

FIG. 53 illustrates the results of exposing patient-derived neurons to chronic doses of an anticonvulsant. A frequency of spiking is plotted over unitless scaled stimulus strength. After exposure to STR in DMSO (versus exposure to the DMSO alone), spike frequency is decreased. A visual comparison of FIG. 52 to FIG. 53 suggests that acute STR has a more severe impact on spike frequency than chronic exposure. Thus it can be seen that methods and compositions of the invention may be used to evaluate signal propagation between cells such as cardiomyocytes or neurons.

1. A method for analyzing cellular signaling, the method comprising:
providing a first cell comprising an optical actuator;
providing a second cell in communication with the first cell, the second cell comprising an optical voltage reporter comprising a variant of archaerhodopsin 3 that includes one or more of the mutations P60S, T80S, D95H, D106H, and F161V relative to wild-type archaerhodopsin 3;
exposing the first cell to a stimulus;
detecting an optical signal from the second cell; and
evaluating the optical signal, thereby characterizing signal propagation from the first cell to the second cell.

2. The method of claim 1, wherein the optical actuator is a light-gated ion channel.

3. The method of claim 2, wherein the stimulus is illumination and the detected optical signal results at least in part from an action potential propagating in the second cell.

4. The method of claim 3, wherein the illumination is spatially-resolved to specifically target the first cell.

5. The method of claim 3, wherein the second cell is a neuron or a cardiomyocyte.

6. The method of claim 3, wherein the first cell and the second cell are among a cluster of neurons when exposed to the stimulus, and further wherein detecting the optical signal includes using a microscope to detect a plurality of signals from the cluster of cells and using a computer system to isolate the optical signal of the second cell from among the plurality of signals.

7. The method of claim 6, wherein the computer system isolates the optical signal by performing an independent component analysis and identifying a spike train from the second cell.

8. The method of claim 6, wherein the second cell also comprises an optical reporter of intracellular calcium.

9. The method of claim 8, wherein the optical reporter of intracellular calcium comprises one selected from the group consisting of R-GECO1, RCaMP2, jRCaMP1a, and jRGECO1a.

10. The method of claim 8, wherein the optical reporter of intracellular calcium and the optical voltage reporter are provided together as a fusion protein.

11. The method of claim 10, wherein the light-gated ion channel is an algal channelrhodopsin.

12. The method of claim 11, further comprising detecting a change in AP waveform and a change in the intracellular calcium level for the second cell upon exposing the first cell to the stimulus.

13. The method of claim 12, further comprising obtaining a sample cell from a person, converting the sample cell into the second cell, and providing the second cell with the variant of archaerhodopsin 3.

14. The method of claim 11, further comprising
providing a third cell comprising the optical actuator;
providing a fourth cell in communication with the third cell, the fourth cell comprising an optical voltage reporter, and further wherein the fourth cell comprises a genetic mutation relative to the second cell;
exposing the third cell to a stimulus;
detecting an second optical signal from the fourth cell, wherein the optical signal and the second optical signal represent changes in membrane potential and intracellular calcium levels; and
comparing, using the computer system, the second optical signal to the optical signal to determine an effect of the genetic mutation on signal propagation.

15. The method of claim 14, wherein the signal from the optical voltage reporter comprises light that does not stimulate the first cell.

16. The method of claim 3, wherein the method further comprises exposing the cells to an agent.

17. The method of claim 16, wherein the method further comprises repeating the exposing, detecting, and evaluating steps before and after exposing the cells to the agent.

18. The method of claim 3, wherein the first cell and the second cell are in synaptic communication through at least one intermediate cell.

19-42. (canceled)
